(19) United States
(12) Patent Application Publication
(10) Pub. No.: US 2008/0234299 A1
(43) Pub. Date: Sep. 25, 2008

(54) Quinazolinones

(75) Inventors: Hans-Peter Buchstaller, Griesheim (DE); Dirk Finsinger, Darmstadt (DE); Kai Schiemann, Seeheim-Jugenheim (DE); Ulrich Emde, Darmstadt (DE)

Correspondence Address: BAKER & DANIELS LLP, 805 15TH STREET, NW, SUITE 700, WASHINGTON, DC, 20005, US

(73) Assignee: MERCK PATENT GMBH, Darmstadt (DE)

(21) Appl. No.: 11/913,149

(22) PCT Filed: Mar. 17, 2006
(86) PCT No.: PCT/EP2006/004655

§ 371 Date: Oct. 30, 2007

(30) Foreign Application Priority Data
May 25, 2005 (DE) 10 2005 024 017.8

(51) Int. Cl. A61K 31/517; C07D 239/88; A61P 35/00

(52) U.S. Cl. 514/266.3; 544/287

(57) ABSTRACT

Compounds of the formula (I), in which R, R¹, R², R³, R⁴, R⁵, R⁶, R⁷, R⁸, Z¹, Z², Z³, k and Y¹ have the meanings indicated in claim 1, can be employed, inter alia, for the treatment of tumours.

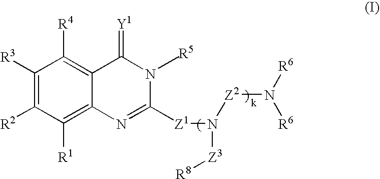

The invention had the object of finding novel compounds having valuable properties, in particular those which can be used for the preparation of medicaments.

The present invention relates to compounds of the formula I and to the use thereof for the treatment and prophylaxis of diseases in which the inhibition, regulation and/or modulation of mitotic motor proteins, in particular the mitotic motor protein Eg5, plays a role, furthermore to pharmaceutical compositions which comprise these compounds.

In detail, the present invention relates to compounds of the formula I which preferably inhibit, regulate and/or modulate one or more mitotic moter proteins, to compositions which comprise these compounds, and to methods for the use thereof for the treatment of diseases and complaints such as angiogenesis, cancer, tumour formation, growth and propagation, arteriosclerosis, ocular diseases, choroidal neovascularisation and diabetic retinopathy, inflammatory diseases, arthritis, neurodegeneration, restenosis, wound healing or transplant rejection. In particular, the compounds according to the invention are suitable for the therapy or prophylaxis of cancer diseases.

During mitosis, various kinesins regulate the formation and dynamics of the spindle apparatus, which is responsible for correct and coordinated alignment and separation of the chromosomes. It has been observed that specific inhibition of a mitotic motor protein—Eg5—results in collapse of the spindle fibres. The result of this is that the chromosomes can no longer be distributed correctly over the daughter cells. This results in mitotic arrest and can consequently cause cell death. Upregulation of the motor protein Eg5 has been described, for example, in tissue from breast lung and colon tumours. Since Eg5 takes on a mitosis-specific function, it is principally rapidly dividing cells and not fully differentiated cells that are affected by Eg5 inhibition. In addition, Eg5 regulates exclusively the movement of mitotic microtubuli (spindle apparatus) and not that of the cytoskeleton. This is crucial for the side-effect profile of the compounds according to the invention since, for example, neuropathies, as observed in the case of Taxol, do not occur or only do so to a weakened extent. The inhibition of Eg5 by the compounds according to the invention is therefore a relevant therapy concept for the treatment of malignant tumours.

In general, all solid and non-solid tumours can be treated with the compounds of the formula I, such as, for example, monocytic leukaemia, brain, urogenital, lymphatic system, stomach, laryngeal and lung carcinoma, including lung adenocarcinoma and small-cell lung carcinoma. Further examples include prostate, pancreatic and breast carcinoma.

Surprisingly, it has been found that the compounds according to the invention effect specific inhibition of mitotic motor proteins, in particular Eg5. The compounds according to the invention preferably exhibit an advantageous biological activity which can easily be detected in the assays described herein, for example. In such assays, the compounds according to the invention preferably exhibit and cause an inhibiting effect, which is usually documented by IC₅₀ values in a suitable range, preferably in the micromolar range and more preferably in the nanomolar range.

As discussed herein, effects of the compound according to the invention are relevant to various diseases. Accordingly, the compounds according to the invention are useful in the prophylaxis and/or treatment of diseases which are influenced by inhibition of one or more mitotic motor proteins, in particular Eg5.

The present invention therefore relates to compounds according to the invention as medicaments and/or medicament active ingredients in the treatment and/or prophylaxis of the said diseases and to the use of compounds according to the invention for the preparation of a pharmaceutical for the treatment and/or prophylaxis of the said diseases, and also to a method for the treatment of the said diseases comprising the administration of one or more compounds according to the invention to a patient in need of such an administration.

It can be shown that the compounds according to the invention have an advantageous effect in a xenotransplant tumour model.

In addition, the compounds according to the invention preferably exhibit one or more advantageous or improved properties compared with the compounds of the prior art. These preferably include an optimised solubility behaviour, a modified, in particular improved, pharmacokinetic behaviour, a modified, in particular improved, metabolitic behaviour or metabolite pattern, a modified, in particular improved, side-effect or tolerance profile and/or a modified half-value period, preferably an extended half-value period.

The host or patient can belong to any mammal species, for example a primate species, particularly humans; rodents, including mice, rats and hamsters; rabbits; horses, cattle, dogs, cats, etc. Animal models are of interest for experimental investigations, providing a model for the treatment of a human disease.

The susceptibility of a certain cell to treatment with the compounds according to the invention can be determined by testing in vitro. Typically, a culture of the cell is combined with a compound according to the invention at various concentrations for a periodine which is sufficient to enable the active ingredients to induce cell death or inhibit migration, usually between approximately one hour and one week. For testing in vitro, cultivated cells from a biopsy sample can be used. The viable cells remaining after the treatment are then counted.

The dose varies depending on the specific compound used, the specific disease, the patient status, etc. Typically, a therapeutic dose is sufficient considerably to reduce the undesired cell population in the target tissue, while the viability of the patient is maintained. The treatment is generally continued until a considerable reduction has occurred, for example at least about a 50% reduction in the cell burden, and can be continued until essentially no undesired cells are detected in the body.

The invention relates to compounds of the formula I

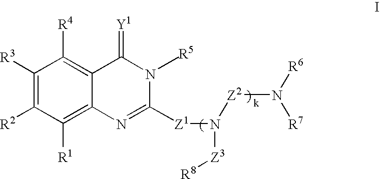

in which
R¹, R², R³ and R⁴, independently of one another, denote H, A, Ar, Het, OR^{a}, SR^{a}, OAr, SAr, N(R^{a})₂, NR^{a}Ar, Hal, NO₂, CN, (CH₂)ₘCOOR^{a}, (CH₂)ₘCOOAr, (CH₂)ₘCON(R^{a})₂, (CH₂)ₘCONHAr, COR^{a}, COAr, S(O)ₘA, S(O)ₘAr, NHCOA, NHCOAr, NHSO₂A, NHSO₂Ar or SO₂N(R^{a})₂,
R^{a} denotes H, A, Ar, Het, aralkyl or heteroaralkyl,
R⁵, R⁸, independently of one another, denote H, A, Ar, Het, aralkyl or heteroaralkyl, and
R⁶, R⁷, independently of one another, denote H, A, Ar, Het, aralkyl or heteroaralkyl, or
R⁶ and R⁷, together with the N atom to which they are bonded, form a saturated or unsaturated 5-, 6- or 7-membered heterocycle, which may optionally contain 1, 2 or 3 further heteroatoms selected from N, S and O,
Y¹ denotes O, S or NR¹,
Z¹, Z², independently of one another, are selected from (CR⁹R¹⁰)ₙ and (CR⁹R¹⁰)ₚ—(C═Y²)—(CR¹¹R¹²)_{q},
Z³ is absent or is selected independently from the meanings indicated for Z¹ and Z²,
A denotes alkyl or cycloalkyl,
Ar denotes aryl or heteroaryl,
Het denotes heteroaryl or heterocyclyl,
Hal denotes F, Cl, Br or I,
Y² denotes O, S or NR²,
R⁹, R¹⁰, R¹¹, R¹², independently of one another, denote H, A, OA, Ar, Het, aralkyl or heteroaralkyl,
k denotes 0, 1 or 2, preferably 0 or 1,
m denotes 1, 2, 3 or 4, preferably 0, 1, 2 or 3,
n denotes 1, 2, 3, 4, 5 or 6, preferably 2, 3, 4 or 5, and
p, q, independently of one another, denote 0, 1, 2, 3 or 4, preferably 0, 1, 2 or 3,
   and pharmaceutically usable derivatives, solvates, tautomers, salts and stereoisomers thereof, including mixtures thereof in all ratios.

The invention also relates to the optically active forms, the enantiomers, the racemates, the diastereomers and the hydrates and solvates of these compounds. The term solvates of the compounds is taken to mean adductions of inert solvent molecules onto the compounds of the formula I which form owing to their mutual attractive force. solvates are, for example, mono- or dihydrates or alkoxides.

Pharmaceutically usable derivatives are preferably taken to mean, for example, the salts of the compounds according to the invention and also so-called prodrug compounds or prodrug derivatives. Suitable prodrug compounds or prodrug derivatives and processes for the preparation thereof are known to the person skilled in the art.

Prodrug derivatives are taken to mean compounds of the formula I which have been modified by means of, for example, alkyl or acyl groups, sugars or oligopeptides and which are rapidly cleaved in the organism to form the effective compounds according to the invention. These also include biodegradable polymer derivatives of the compounds according to the invention, as described, for example, in Int. J. Pharm. 115, 61-67 (1995).

Similar compounds are described, for example, in Tetrahedron Lett. 1988, 29, 5855-5858, Tetrahedron Lett. 2003, 44, 217-219, J. Org. Chem. 1997, 62, 4880-4882, J. Org. Chem. 1999, 64, 6462-6467, Chem. Lett. 1995, 423-424, J. Org. Chem. 2000, 65, 5009-5013, Chem. Lett. 2003, 32, 222-223, US2003149069A1, but are not mentioned in connection with cancer treatments and/or do not contain the features essential to the invention.

The expression “effective amount” denotes the amount of a medicament or of a pharmaceutical active ingredient which causes in a tissue, system, animal or human a biological or medical response which is sought or desired, for example, by a researcher or physician. In addition, the expression “therapeutically effective amount” denotes an amount which causes at least one of the following effects in a human or another mammal (compared with a subject who has not received this amount):

improvement in the healing treatment, healing, prevention or elimination of a disease, syndrome, condition, complaint, disorder or side-effects or also the reduction in the progress of a disease, condition or disorder. The term “therapeutically effective amount” also encompasses the amounts which are effective for increasing or enhancing normal physiological function.

For the purposes of the present invention, the term “herein” preferably denotes “in the description and/or claims” and in particular “above and/or below in the description and/or claims”. Thus, for example, the expression “as described herein” preferably has the meaning “as described in the description and/or claims” and in particular the meaning “as described above and/or below in the description and/or claims”.

The invention also relates to the use of mixtures of the compounds according to the invention, for example mixtures of two diastereomers, for example mixtures of two diastereomers in the ratio of about 1:1, 1:2, 1:3, 1:4, 1:5, 1:10, 1:100 or about 1:1000.

These are particularly preferably mixtures of stereoisomeric compounds.

The invention relates to the compounds of the formula I and salts thereof and to a process for the preparation of compounds of the formula I according to the patent claims and pharmaceutically usable derivatives, salts, solvates and stereoisomers thereof, characterised in that
a) a compound of the formula II

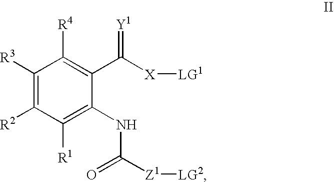

in which R¹, R², R³, R⁴, R⁵, Y¹ and Z¹ have the meanings indicated herein, X stands for O NH or S and in particular for O, and LG¹ and LG² each stands for a leaving group, is cyclised with removal of the leaving group LG¹ to give a compound of the formula IIb

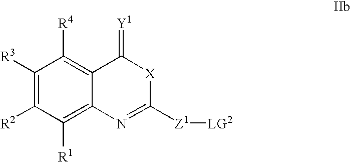

b) the compound of the formula IIb is reacted with a compound of the formula III

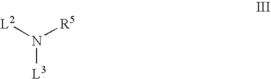

in which R⁵ has the meaning indicated herein, and L² and L³, independently of one another, stand for H or a metal atom and in particular both stand for H,
giving a compound of the formula IIc

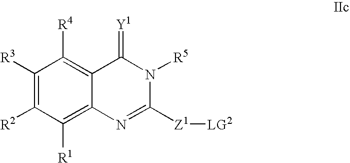

c) the compound of the formula IIc is reacted with a compound of the formula IV

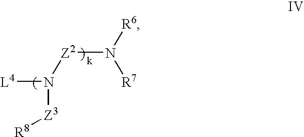

in which L⁴ stands for H or a metal atom, and Z², Z³, k, R⁶, R⁷ and R⁸ have the meanings indicated herein,
where the groups LG² and L⁴ are removed, giving a compound of the formula I; and optionally
d) the resultant compound of the formula I is isolated and/or treated with an acid or base in order to convert it into one of its salts.

Leaving groups LG¹ and LG² which are suitable for the above process are known to the person skilled in the art, for example from standard works, such as Houben-Weyl, Methoden der organischen Chemie [Methods of Organic Chemistry], Georg-Thieme-Verlag, Stuttgart.

LG¹ preferably stands for a leaving group selected from H and a metal atom. LG¹ particularly preferably stands for H, in particular if X stands for O.

LG² preferably stands for a leaving group selected from Hal, in particular Cl, Br or I, and OSO₂R^{e}, in which R^{e} is preferably selected from

A, in particular alkyl, such as methyl and trifluoromethyl, and
Ar, in particular phenyl or substituted phenyl.

LG² particularly preferably stands for Hal, particularly preferably Cl, Br or I, and in particular for Br.

L² and L³ preferably stand, independently of one another, for H or a metal atom, for example for an alkali metal atom, such as Na or K, and particularly preferably for H.

The process according to the invention is preferably carried out under reaction conditions which are known and suitable for the said reactions or can be derived in a simple manner from analogous reactions by the person skilled in the art. Use may also be made here of variants known per se which are not mentioned here in greater detail.

The starting materials for the process according to the invention are either commercial or can be prepared by methods known per se, as described in the literature (for example in the standard works, such as Houben-Weyl, Methoden der organischen Chemie [Methods of Organic Chemistry], Georg-Thieme-Verlag, Stuttgart), to be precise under reaction conditions which are known and suitable for these reactions. Use may also be made here of variants known per se which are not mentioned here in greater detail.

If desired, the starting materials can also be formed in situ, so that they are not isolated from the reaction mixture, but instead are immediately converted further into the compounds according to the invention.

It is furthermore possible to convert a compound of the formula I into another compound of the formula I by converting one or more radical(s), for example one or more radicals selected from R¹, R², R³, R⁴, R⁵, R⁵, R⁶, R⁷ and R⁸, into one or more other radicals R¹, R², R³, R⁴, R⁵, R⁵, R⁶, R⁷ and R⁸ (i.e., for example, converting a radical R¹ into another radical R¹ or a radical R⁵ into another radical R⁵), for example by reducing nitro groups, for example by hydrogenation on Raney nickel or Pd/carbon in an inert solvent, such as

methanol or ethanol, to amino groups and/or
converting an ester group into a carboxyl group and/or
converting an amino group into an alkylated amine by reductive amination and/or esterifying carboxyl groups by reaction with alcohols.

Furthermore, free amino groups can be acylated in a conventional manner using an acid chloride or anhydride or alkylated using an unsubstituted or substituted alkyl halide, advantageously in an inert solvent, such as dichloromethane or THF, and/or in the presence of a base, such as triethylamine or pyridine, at temperatures between −60 and +30°.

If desired, a functionally modified amino and/or hydroxyl group in a compound of the formula I can be liberated by solvolysis or hydrogenolysis by conventional methods. This can be carried out, for example, using NaOH or KOH in water, water/THF or water/dioxane at temperatures between 0 and 100°.

The invention furthermore relates to a preferred process for the preparation of compounds of the formula I according to the patent claims and pharmaceutically usable derivatives, salts, solvates and stereoisomers thereof, characterised in that
a) a compound of the formula II′

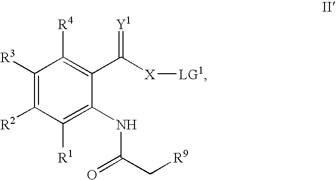

in which R¹, R², R³, R⁴, R⁵, R⁹, X, LG¹ and Y¹ have the meanings indicated herein, LG¹ stands for a leaving group,
is cyclised with removal of the leaving group LG¹ to give a compound of the formula IIb

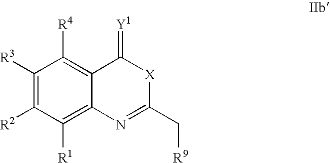

b) the compound of the formula IIb′ is converted by reaction with a compound of the formula III

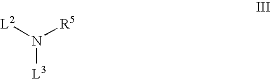

and introduction of a leaving group LG³, into a compound of the formula IIc′

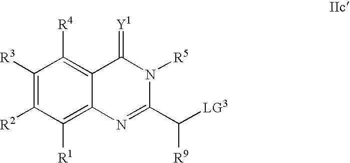

c1) the compound of the formula IIc′ is converted by reaction with cyanide into a compound of the formula IId′

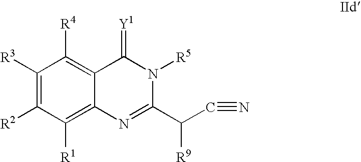

c2) the compound of the formula IId′ is converted under reductive conditions into a compound of the formula I′, i.e. into a compound of the formula I in which k is equal to 0, Z¹ stands for —CHR⁹—CH₂—, and R⁶ and R⁷ each stands for H;

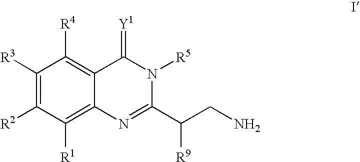

and optionally either
c3a) the compound of the formula I′ obtained in step c2) is converted by reaction with a compound FG¹-R⁶ and/or FG²-R⁷ into a compound of the formula I″, i.e. a compound of the formula I in which k is equal to 0, Z¹ stands for —CHR⁹—CH₂— and in which R⁶ and/or R⁷ are different from H; or

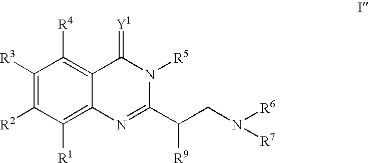

c3b) the compound of the formula I′ obtained in step c2) is converted by reaction with a compound of the formula FG³-Z²-NR⁶R⁷ and optionally a compound FG⁴-Z³-R⁸, in which FG³ and FG⁴ stand for suitable functional groups, into a compound of the formula I′″

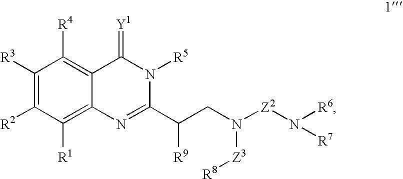

i.e. a compound of the formula I in which k is equal to 1, Z¹ stands for —CHR⁹—CH₂— and in which Z³-R⁸ optionally stands for a group other than H;
and optionally
d) the resultant compound of the formula I (or of the formula I′, formula I″ or formula I′″) is isolated and/or treated with an acid or base in order to convert it into one of its salts.

The preferred process according to the invention is preferably carried out under reaction conditions which are known and suitable for the said reactions or can be derived in a simple manner from analogous reactions by the person skilled in the art. Use may also be made here of variants known per se which are not mentioned here in greater detail. The starting materials for the process according to the invention are either commercial or can be prepared by methods known per se, as described in the literature.

The radicals R¹, R², R³, R⁴, R⁵, R⁶, R⁷, R⁸, R⁹, X, Z², Z³, L², L³, LG¹, LG³ and Y¹ in this preferred process also preferably have the meanings indicated herein, unless explicitly indicated otherwise in the individual steps.

Leaving groups LG³ which are suitable for the above process are known to the person skilled in the art, for example from standard works, such as Houben-Weyl, Methoden der organischen Chemie [Methods of Organic Chemistry], Georg-Thieme-Verlag, Stuttgart.

LG³ preferably stands for a leaving group selected from Hal, in particular Cl, Br or I, and OSO₂R^{e}, in which R^{e} is preferably selected from

A, in particular alkyl, such as methyl and trifluoromethyl, and
Ar, in particular phenyl or substituted phenyl.

LG³ particularly preferably stands for Hal, particularly preferably Cl, Br or I, and in particular for Br.

Functional groups FG¹, FG², FG³ and FG⁴ of this type which are suitable for the above process are known to the person skilled in the art, for example from standard works, such as Houben-Weyl, Methoden der organischen Chemie [Methods of Organic Chemistry], Georg-Thieme-Verlag, Stuttgart.

For the purposes of the process according to the invention, the term FG¹, FG², FG³ and FG⁴ preferably each stands, independently of one another, for a functional group which is suitable for the reaction with a primary amino group, i.e. convert a primary amino group into a secondary (or a secondary into a tertiary) amino group.

FG¹, FG², FG³ and FG⁴ particularly preferably stand for the functional groups present in alkylating agents, arylating agents and/or acylating agents, and for functional groups which are suitable for a reductive amination.

If FG¹, FG², FG³ and/or FG⁴ stand for the functional group of alkylating agents, arylating agents and/or acylating agents, FG¹, FG², FG³ and/or FG⁴ preferably stand, independently of one another, for Hal, in particular Cl, Br or I, and OSO₂R^{e}, in which R^{e} is preferably selected from A, in particular alkyl, such as methyl and trifluoromethyl, and Ar, in particular phenyl or substituted phenyl, and particularly preferably for Hal, in particular Cl and/or Br.

If FG¹, FG², FG³ and/or FG⁴ stand for functional groups which are suitable for a reductive amination, they are preferably, independently of one another, selected as below:

If R⁶ and/or R⁷ represent a radical which is bonded to the nitrogen atom of the group NR⁶R⁷ via a methylene group or a methyne group, FG¹ and/or FG² preferably stands for an oxo radical, i.e. for ═O. For example, a group —NR⁶R⁷ in which R⁶ and R⁷ stand for H can be converted firstly into a group of the formula —N(═CR^{f}R^{g})R⁷ by reaction with a compound of the formula R^{f}—C═O—R^{g}, in which R^{f} and R^{g} are preferably, independently of one another, selected from H, A, aryl and Het, and subsequently into a group of the formula —N(CHR^{f}R^{g})R⁷ under reductive conditions. A further reaction of a group of the formula —N(CHR^{f}R^{g})R⁷ with a compound of the formula R^{f}—C═O—R^{g} or of the formula R^{h}C═O—Rⁱ (in which in which R^{h} and Ri^{g} are preferably, independently of one another, selected from H, A, aryl and Het) leads analogously to a group of the formula N(CHR^{f}R^{g})₂ or N(CHR^{f}R^{g})(CHR^{h}Rⁱ), in each case depending on the starting material employed. This reaction sequence is known as reductive amination and is described in detail in the prior art, for example in such as Houben-Weyl, Methoden der organischen Chemie [Methods of Organic Chemistry], Georg-Thieme-Verlag, Stuttgart.

Analogously to the methods described above, a group R⁸-Z³ which is different from H can also optionally be introduced into the compounds of the formula I (see step c3b) in the preferred process), i.e. a group of the formula -Z¹-NH-Z²- can be converted into a group of the formula -Z¹-N(Z³-R⁸)-Z²-.

A reductive amination of this type is preferably carried out using compounds of the formula R^{f}—C═O—R^{g} and/or or of the formula R^{h}—C═O—Rⁱ, and in particular compounds of the formula R^{f}—C═O—H (i.e. compounds of the formula R^{f}— CHO) or of the formula R^{f}—C═O—H (i.e. compounds of the formula R^{f}—CHO), in which R^{f} and Rⁱ are as defined above and in particular are selected from A, aryl and Het. For the provision of reductive conditions, conventional reducing agents, such as, for example, hydrides, preferably cyanoborohydrides and in particular NaCNBH₃, can advantageously be employed here.

For the preparation of compounds of the formula I in which k stands for 1 or 2 and in which R⁶ and/or R⁷ stands for H, it may be advantageous to employ compounds of the formula FG³-Z²-NR⁶R⁷, in which R⁶ and/or R⁷ stands for an amino-protecting group, in the preferred process in reaction step c3b), and subsequently to convert the resultant compound of the formula I in which R⁶ and/or R⁷ stands for an amino-protecting group then into a compound of the formula I in which R⁶ and/or R⁷ stand for H by removal of the amino-protecting group(s). To this end, use can advantageously be made of compounds of the formula FG³-Z²-NR⁶R⁷ in which R⁷ stands for the Boc protecting group (Boc stands for tert-butoxycarbonyl) and R⁶ stands for H. Further suitable amino-protecting groups are known to the person skilled in the art, for example from Houben-Weyl, Methoden der organischen Chemie [Methods of Organic Chemistry], Georg-Thieme-Verlag, Stuttgart (see, inter alia, Houben-Weyl, 15/1, 117-133, and Greene, Protective Groups in Organic Syntheses, New York: Wiley 1981).

For the purposes of the invention, cyanides are both prussic acid (HCN) and also compounds and precursors which liberate prussic acid or cyanide anions, and in particular the salts of prussic acid, very particularly preferably potassium cyanide (KCN) and sodium cyanide (NaCN).

Suitable conditions for carrying out the process steps of the process according to the invention are known to the person skilled in the art, for example from Houben-Weyl, Methoden der organischen Chemie [Methods of Organic Chemistry], Georg-Thieme-Verlag, Stuttgart.

The process steps of the process according to the invention are preferably carried out in under the respective reaction conditions in inert solvents. For the purposes of the invention, suitable inert solvents are, for example, hydrocarbons, such as hexane, petroleum ether, benzene, toluene or xylene; chlorinated hydrocarbons, such as trichloroethylene, 1,2-dichloroethane, carbon tetrachloride, chloroform or dichloromethane; alcohols, such as methanol, ethanol, isopropanol, n-propanol, n-butanol or tert-butanol; ethers, such as diethyl ether, diisopropyl ether, tetrahydrofuran (THF) or dioxane; glycol ethers, such as ethylene glycol monomethyl or monoethyl ether, ethylene glycol dimethyl ether (diglyme); ketones, such as acetone or butanone; amides, such as acetamide, dimethylacetamide or dimethylformamide (DMF); nitriles, such as acetonitrile; sulfoxides, such as dimethyl sulfoxide (DMSO); carbon disulfide; carboxylic acids, such as formic acid or acetic acid; nitro compounds, such as nitromethane or nitrobenzene; esters, such as ethyl acetate, water, or mixtures of the said solvents. The process steps of the process according to the invention are generally at reaction temperatures in the range from −20° C. to +200° C., preferably in the range from 0° C. to 150° C., for example at about 0° C., at about room temperature (25° C.), at about 40° C., at about 50° C., at about 65° C. or at about 130° C.

In general, the reaction times for the process steps of the process according to the invention are in the range from a few minutes to a few days, preferably in the range from ten minutes to 48 hours, in particular one hour to twelve hours.

Suitable reductive conditions for carrying out the process according to the invention are known to the person skilled in the art, for example from Houben-Weyl, Methoden der organischen Chemie [Methods of Organic Chemistry], Georg-Thieme-Verlag, Stuttgart. Preferred reductive conditions for the purposes of the process according to the invention are the reaction with hydrides as reducing agents, for example metal or boron hydrides and in particular complex hydrides, such as lithium aluminium hydride, and so-called deactivated complex hydrides, such as LiAl(OR)ₓH₄₋ₓ, in which X stands for 1, 2 or 3 and R stands for alkyl radicals or alkoxyalkyl radicals having 1 to 5 carbons and preferably for alkyl radicals having 1 to 4 carbons or for alkoxyalkyl radicals having 2 to 4 carbons. A deactivated complex hydride which is suitable for this reduction is commercially available under the name Vitride. A particularly preferred form for reductive conditions, in particular for the conversion of compounds of the formula IIc′ into compounds of the formula I′, is hydrogenation in a hydrogen atmosphere in the presence of suitable hydrogenation catalysts, such as, for example, a platinum metal catalyst, in particular palladium/carbon, or a nickel metal catalyst, in particular Raney nickel (Raney Ni). The reaction is preferably carried out under reductive conditions in a solvent which is inert under the reaction conditions.

The hydrogenation is preferably carried out in a polar, inert solvent, such as, for example, methanol or THF. In many cases, the hydrogenation using Raney nickel can advantageously be carried out in THF as solvent in the presence of trifluoroacetic acid (TFA) or in methanol as solvent in the presence of ammonia.

The cyclisation of a compound of the formula II to give a compound of the formula IIb (or of a compound of the formula II′ into a compound of the formula IIb′) can advantageously be carried out by heating in acetic anhydride, preferably to a temperature in the region of the boiling point of acetic anhydride, preferably for a reaction duration in the range from two to five hours.

The mixtures of diastereomers and enantiomers of the compounds of the formula I which may be obtained by the process described herein are preferably resolved by chromatography or crystallisation.

If desired, the bases and acids of the formula I obtained by the process described herein are converted into their salts.

Above and below, the radicals R¹, R², R³, R⁴, R^{a}, R⁵, R⁶, R⁷, R⁸, R⁹, R¹⁰, R¹¹, R¹², Y¹, Y², Z¹, Z², Z³, A, Het, Ar, Hal, k, m, n, p and q have the meanings indicated for the formula I, unless expressly indicated otherwise. If individual radicals occur more than once within a compound, the radicals, independently of one another, adopt the meanings indicated.

Preferred compounds of the formula I are compounds of the formula Iα

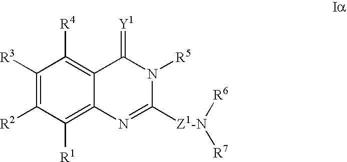

in which
R¹, R², R³, R⁴, R⁵, R⁶, R⁷, Y¹ and Z¹ are as defined herein,
and pharmaceutically usable derivatives, solvates, tautomers, salts and stereoisomers thereof, including mixtures thereof in all ratios.

Preferred compounds of the formula Iα are compounds as defined above in which R¹, R², R³, R⁴, R⁵ and Y¹ have the meanings indicated herein and in which
Z¹ stands for (CR⁹R¹⁰)ₙ or (CR⁹R¹⁰)ₚ—(C═Y²)—(CR¹¹R¹²)_{q} and preferably stands for (CHR¹⁰)ₙ or (CHR¹⁰)ₚ—(C═Y²)—(CR¹¹H)_{q}, in which
   n preferably stands for 2, 3 or 4, preferably 2 or 3, and
   p and q, independently of one another, preferably stand for 0, 1 or 2, preferably 0 or 1, where one of the two indices, p or q, preferably stands for 0, and
R⁶, R⁷, independently of one another, are selected from H, A, Ar, Het, aralkyl and heteroaralkyl and preferably, independently of one another, from H, A, aralkyl and heteroaralkyl.

Preferred compounds of the formula I are compounds of the formula Iβ

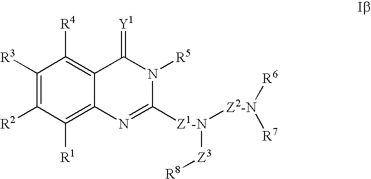

in which
R¹, R², R³, R⁴, R⁵, R⁶, R⁷, R⁸, Y¹, Z¹, Z² and Z³ are as defined herein,
and pharmaceutically usable derivatives, solvates, tautomers, salts and stereoisomers thereof, including mixtures thereof in all ratios.

Preferred compounds of the formula Iβ are compounds as defined above in which R¹, R², R³, R⁴, R⁵ and Y¹ have the meanings indicated herein and in which
Z¹ stands for (CR⁹R¹⁰)ₙ or (CR⁹R¹⁰)ₚ—(C═Y²)—(CR¹¹R¹²)_{q} and preferably stands for (CHR¹⁰)ₙ or (CHR¹⁰)ₚ—(C═Y²)—(CR¹¹H)_{q}, in which
   n preferably stands for 1, 2 or 3, preferably 1 or 2, and
   p and q, independently of one another, preferably stand for 0, 1 or 2, preferably 0 or 1, where one of the two indices, p or q, preferably stands for 0, and
R⁶, R⁷, independently of one another, are selected from H, A, Ar, Het, aralkyl and heteroaralkyl and preferably, independently of one another, from H, A, aralkyl and heteroaralkyl,
   and pharmaceutically usable derivatives, solvates, tautomers, salts and stereoisomers thereof, including mixtures thereof in all ratios.

For the purposes of the invention, alkyl is preferably unbranched (linear) or branched, has 1, 2, 3, 4, 5, 6, 7, 8, 9 or 10 C atoms and may be substituted. Substituted alkyl is preferably an alkyl radical as described in this section which has 1-7, preferably 1-5 and particularly preferably 1-3 substituents, which are preferably selected from ═O, ═S; ═NH, ═N-alkyl, Hal, in particular Cl and F, OH, O-alkyl, NH₂ and N(alkyl)₂, in which alkyl is as described above and is preferably unsubstituted alkyl as described above. Substituted alkyl particularly preferably denotes an alkyl radical as described above in which 1-7H atoms have been replaced by F and/or chlorine, for example a perchlorinated or perfluorinated alkyl radical. Fluorine- and/or chlorine-substituted alkyl radicals of this type preferably have 1, 2, 3, 4 or 5 C atoms. Preferred fluorine- and/or chlorine-substituted alkyl radicals are perfluorinated alkyl radicals, in particular trifluoromethyl radicals. Unsubstituted or substituted alkyl particularly preferably denotes methyl, furthermore ethyl, propyl, isopropyl, butyl, isobutyl, sec-butyl or tert-butyl, furthermore also pentyl, 1-, 2- or 3-methylbutyl, 1,1-, 1,2- or 2,2-dimethylpropyl, 1-ethylpropyl, hexyl, 1-, 2-, 3- or 4-methylpentyl, 1,1-, 1,2-, 1,3-, 2,2-, 2,3- or 3,3-dimethylbutyl, 1- or 2-ethylbutyl, 1-ethyl-1-methylpropyl, 1-ethyl-2-methylpropyl, 1,1,2- or 1,2,2-trimethylpropyl, further particularly preferably trifluoromethyl. Alkyl very particularly preferably denotes an alkyl radical having 1, 2, 3, 4, 5 or 6 C atoms, which may be chlorinated and/or fluorinated as described above, and is, in particular, selected from methyl, ethyl, propyl, isopropyl, butyl, isobutyl, sec-butyl, tert-butyl, pentyl, hexyl, trifluoromethyl, pentafluoroethyl and 1,1,1-trifluoroethyl. In addition, alkyl preferably stands for an alkyl radical having 1, 2, 3, 4, 5 or 6 C atoms, particularly preferably 1, 2 or 3 C atoms, which has 1 or 2, preferably 1, substituent(s) selected from ═O, ═S; ═NH and ═N-alkyl, preferably from ═O, ═S and ═NH and in particular from ═O and ═S. Alkyl then particularly preferably stands for carbonyl, acetyl, propionyl or butyryl (butanoyl) and the thio derivatives thereof.

For the purposes of the invention, cycloalkyl is preferably selected from substituted or unsubstituted cyclopropyl, cyclobutyl, cyclopentyl, cyclohexyl and cycloheptyl. Substituted cycloalkyl is preferably a cycloalkyl radical as described above which has 1-7, preferably 1-5 and particularly preferably 1-3 substituents, which are preferably selected from Hal, in particular Cl and F, OH, O-alkyl, NH₂ and N(alkyl)₂, in which alkyl is as described above and is preferably unsubstituted alkyl as described above.

For the purposes of this invention, alkylene is preferably an unbranched or branched divalent hydrocarbon radical having 1-10 C atoms, preferably 1-4 C atoms, which may optionally have 1-7, preferably 1-5 and particularly preferably 1-3 substituents, which are preferably selected from ═O, ═S; ═NH, ═N-alkyl, Hal, in particular Cl and F, OH, O-alkyl, NH₂ and N(alkyl)₂, in which alkyl is as described above and is preferably unsubstituted alkyl as described above. Unsubstituted alkylene preferably stands for methylene, ethylene, n-propylene, isopropylene or n-butylene and in particular for methylene or ethylene. Substituted alkylene preferably stands for methylene, ethylene, n-propylene, isopropylene or n-butylene and in particular for methylene or ethylene, which has 1-3 substituents as described above and in particular 1 or 2 substituents selected from ═O, ═S; ═NH and Hal, in particular Cl and F. If substituted alkylene has 1 or 2 substituents selected from ═O, ═S and ═NH, it particularly preferably stands for —C═O—, —CH₂—C═O—, —C═O—CH₂—, —CH₂—CH₂—C═O—, —C═O—CH₂—CH₂— or —CH₂—C═O—CH₂—, very particularly preferably —CH₂—C═O—, —C═O—CH₂—, —CH₂—CH₂—C═O— or —C═O—CH₂—CH₂—, and the thio derivatives thereof.

For the purposes of the invention, aryl is preferably a substituted or unsubstituted benzene ring, for example a phenyl radical, or a system comprising benzene rings, such as, for example, anthracene, phenanthrene or naphthalene ring systems or radicals. Substituted aryl is preferably an aryl radical as described above which has 1-7, preferably 1-5 and particularly preferably 1-3 substituents, which are preferably selected from Hal, A, OH, OA, NH₂, NO₂, CN, COOH, COOA, CONH₂, NHCOA, NHCONH₂, NHSO₂A, CHO, COA, SO₂NH₂, SO₂A, —CH₂—COOH and —OCH₂—COOH and in particular from Hal, in particular Cl and F, OH, O-alkyl, NH₂ and N(alkyl)₂, in which alkyl is as described above and is preferably unsubstituted alkyl as described above.

Aryl therefore particularly preferably denotes phenyl, naphthyl or biphenyl, each of which is unsubstituted or mono-, di- or trisubstituted by Hal, A, OH, OA, NH₂, NO₂, CN, COOH, COOA, CONH₂, NHCOA, NHCONH₂, NHSO₂A, CHO, COA, SO₂NH₂, SO₂A, —CH₂—COOH and/or —OCH₂—COOH.

Aryl very particularly preferably denotes phenyl, o-, m- or p-tolyl, o-, m- or p-ethylphenyl, o-, m- or p-propylphenyl, o-, m- or p-isopropylphenyl, o-, m- or p-tert-butylphenyl, o-, m- or p-hydroxyphenyl, o-, m- or p-methoxyphenyl, o-, m- or p-nitrophenyl, o-, m- or p-aminophenyl, o-, m- or p-(N-methylamino)phenyl, o-, m- or p-(N-methylaminocarbonyl)-phenyl, o-, m- or p-acetamidophenyl, o-, m- or p-methoxyphenyl, o-, m- or p-ethoxyphenyl, o-, m- or p-ethoxycarbonylphenyl, o-, m- or p-(N,N-dimethylamino)phenyl, o-, m- or p-(N,N-dimethylaminocarbonyl)phenyl, o-, m- or p-(N-ethylamino)phenyl, o-, m- or p-(N,N-diethylamino)phenyl, o-, m- or p-fluorophenyl, o-, m- or p-bromophenyl, o-, m- or p-chlorophenyl, o-, m- or p-(methylsulfonamido)phenyl, o-, m- or p-(methylsulfonyl)phenyl, further preferably 2,3-, 2,4-, 2,5-, 2,6-, 3,4- or 3,5-difluorophenyl, 2,3-, 2,4-, 2,5-, 2,6-, 3,4- or 3,5-dichlorophenyl, 2,3-, 2,4-, 2,5-, 2,6-, 3,4- or 3,5-dibromophenyl, 2,4- or 2,5-dinitrophenyl, 2,5- or 3,4-dimethoxyphenyl, 3-nitro-4-chlorophenyl, 3-amino-4-chloro-, 2-amino-3-chloro-, 2-amino-4-chloro-, 2-amino-5-chloro- or 2-amino-6-chlorophenyl, 2-nitro-4-N,N-dimethylamino- or 3-nitro-4-N,N-dimethylaminophenyl, 2,3-diaminophenyl, 2,3,4-, 2,3,5-, 2,3,6-, 2,4,6- or 3,4,5-trichlorophenyl, 2,4,6-trimethoxyphenyl, 2-hydroxy-3,5-dichlorophenyl, p-iodophenyl, 3,6-dichloro-4-aminophenyl, 4-fluoro-3-chlorophenyl, 2-fluoro-4-bromophenyl, 2,5-difluoro-4-bromophenyl, 3-bromo-6-methoxyphenyl, 3-chloro-6-methoxyphenyl, 3-chloro-4-acetamidophenyl, 3-fluoro-4-methoxyphenyl, 3-amino-6-methylphenyl, 3-chloro-4-acetamidophenyl or 2,5-dimethyl-4-chlorophenyl.

For the purposes of the invention, heteroaryl is preferably a substituted or unsubstituted monocyclic 5- to 7-membered aromatic ring or cycle or an unsubstituted or substituted fused ring system comprising two or three monocyclic 5- to 7-membered rings of this type, where the ring or rings contain one or more heteroatoms, preferably selected from N, S and O. A heteroaryl radical preferably contains 1 to 4 heteroatoms as described above and in particular 1 to 4 nitrogen atoms. Examples of heteroaryl radicals are furanyl, thiophenyl, pyrrolyl, imidazolyl, pyrazolyl, triazolyl, tetrazolyl, thiazolyl, oxazolyl, isoxazolyl, oxadiazolyl, oxo-pyridyl, thiadiazolyl, isothiazolyl, pyridyl, pyridazyl, pyrazinyl, pyrimidyl, quinolinyl, isoquinolinyl, benzofuranyl, benzothiophenyl, indolyl, indazolyl, and substituted derivatives thereof, preferably derivatives thereof which are mono-, di- or trisubstituted by Hal, A, OH, OA, NH₂, NO₂, CN, COOH, COOA, CONH₂, NHCOA, NHCONH₂, NHSO₂A, CHO, COA, SO₂NH₂, SO₂A, —CH₂—COOH or —OCH₂—COOH.

Heteroaryl preferably denotes a mono- or bicyclic, aromatic heterocycle having one or more N, O and/or S atoms which is unsubstituted or mono-, di- or trisubstituted by Hal, A, NO₂, NHA, NA₂, OA, COOA and/or CN.

Heteroaryl particularly preferably denotes a monocyclic saturated or aromatic heterocycle having one N, S or O atom, which may be unsubstituted or mono-, di- or trisubstituted by Hal, A, NHA, NA₂, NO₂, COOA and/or benzyl.

Irrespective of further substitutions, unsubstituted heteroaryl denotes, for example, 2- or 3-furyl, 2- or 3-thienyl, 1-, 2- or 3-pyrrolyl, 1-, 2,4- or 5-imidazolyl, 1-, 3-, 4- or 5-pyrazolyl, 2-, 4- or 5-oxazolyl, 3-, 4- or 5-isoxazolyl, 2-, 4- or 5-thiazolyl, 3-, 4- or 5-isothiazolyl, 2-, 3- or 4-pyridyl, 2-, 4-, 5- or 6-pyrimidinyl, furthermore preferably 1,2,3-triazol-1-, -4- or -5-yl, 1,2,4-triazol-1-, -3- or 5-yl, 1- or 5-tetrazolyl, 1,2,3-oxadiazol-4- or -5-yl, 1,2,4-oxadiazol-3- or -5-yl, 1,3,4-thiadiazol-2- or -5-yl, 1,2,4-thiadiazol-3- or -5-yl, 1,2,3-thiadiazol-4- or -5-yl, 3- or 4-pyridazinyl, pyrazinyl, 1-, 2-, 3-, 4-, 5-, 6- or 7-indolyl, 4- or 5-isoindolyl, 1-, 2-, 4- or 5-benzimidazolyl, 1-, 3-, 4-, 5-, 6- or 7-benzopyrazolyl, 2-, 4-, 5-, 6- or 7-benzoxazolyl, 3-, 4-, 5-, 6- or 7-benzisoxazolyl, 2-, 4-, 5-, 6- or 7-benzothiazolyl, 2-, 4-, 5-, 6- or 7-benzisothiazolyl, 4-, 5-, 6- or 7-benz-2,1,3-oxadiazolyl, 2-, 3-, 4-, 5-, 6-, 7- or 8-quinolyl, 1-, 3-, 4-, 5-, 6-, 7- or 8-isoquinolyl, 3-, 4-, 5-, 6-, 7- or 8-cinnolinyl, 2-, 4-, 5-, 6-, 7- or 8-quinazolinyl, 5- or 6-quinoxalinyl, 2-, 3-, 5-, 6-, 7- or 8-2H-benzo-1,4-oxazinyl, further preferably 1,3-benzodioxol-5-yl, 1,4-benzodioxan-6-yl, 2,1,3-benzothiadiazol-4- or -5-yl or 2,1,3-benzoxadiazol-5-yl.

For the purposes of the invention, aralkyl or arylalkyl is preferably an aryl radical as defined above connected to an alkylene radical as defined above. Aralkyl may be substituted or preferably unsubstituted. Examples of preferred unsubstituted arylalkyl radicals are benzyl, phenethyl, phenylpropyl and phenylbutyl and in particular benzyl and phenethyl. Substituted arylalkyl is preferably an arylalkyl radical as described above which has 1-7, preferably 1-5 and particularly preferably 1-3 substituents, which are preferably selected from Hal, A, OH, OA, NH₂, NO₂, CN, COOH, COOA, CONH₂, NHCOA, NHCONH₂, NHSO₂A, CHO, COA, SO₂NH₂, SO₂A, —CH₂—COOH and —OCH₂—COOH and particularly preferably from Hal, in particular Cl and F, OH, O-alkyl, NH₂ and N(alkyl)₂, in which alkyl is as described above and is preferably unsubstituted alkyl as described above. In addition, alkylene in aralkyl preferably stands for an alkylene radical having 1, 2, 3, 4, 5 or 6 C atoms, particularly preferably 1, 2 or 3 C atoms, which has 1 or 2, preferably 1, substituent(s) selected from ═O, ═S; ═NH and ═N-alkyl, preferably from ═O, ═S and ═NH and in particular from ═O and ═S. Aralkyl then particularly preferably stands for benzoyl, 2-phenylacetyl and 3-phenylpropionyl or 4-phenylbutyryl (4-phenylbutanoyl), and the thio derivatives thereof, where these aralkyl radicals may have 1-5 and preferably 1-3 substituents, which are preferably selected from Hal, A, OH, OA, NH₂, NO₂, CN, COOH, COOA, CONH₂, NHCOA, NHCONH₂, NHSO₂A, CHO, COA, SO₂NH₂, SO₂A, —CH₂—COOH and —OCH₂—COOH and particularly preferably from Hal, in particular Cl and F, OH, O-alkyl, NH₂ and N(alkyl)₂, in which alkyl is as described above and is preferably unsubstituted alkyl as described above.

For the purposes of the invention, heteroaralkyl is preferably aralkyl as defined above in which one or more C atoms, preferably 1 to 4 C atoms, have been substituted by heteroatoms, preferably selected from N, S and O and in particular from N and S. Heteroaralkyl is particularly preferably a heteroaryl radical as defined above connected to an alkylene radical as defined above. Heteroaralkyl may be substituted or preferably unsubstituted. Examples of preferred unsubstituted heteroarylalkyl radicals are pyridyl-2-ylmethyl, yridyl-3-ylmethyl, pyridyl-4-ylmethyl, pyridyl-2-ylethyl, pyridyl-3-ylethyl and pyridyl-4-ylethyl. In addition, alkylene in aralkyl preferably stands for an alkylene radical having 1, 2, 3, 4, 5 or 6 C atoms, particularly preferably 1, 2 or 3 C atoms, which has 1 or 2, preferably 1, substituent(s) selected from ═O, ═S; ═NH and ═N-alkyl, preferably from ═O, ═S and ═NH and in particular from ═O and ═S. Aralkyl then particularly preferably stands for pyridyl-2-ylcarbonyl, pyridyl-3-ylcarbonyl, pyridyl-4-ylcarbonyl, pyridyl-2-ylacetyl, pyridyl-3-ylacetyl or pyridyl-4-ylacetyl, and the thio derivatives thereof, where these heteroaralkyl radicals may have 1-5 and preferably 1-3 substituents, which are preferably selected from Hal, A, OH, OA, NH₂, NO₂, CN, COOH, COOA, CONH₂, NHCOA, NHCONH₂, NHSO₂A, CHO, COA, SO₂NH₂, SO₂A, —CH₂—COOH and —OCH₂—COOH and particularly preferably from Hal, in particular Cl and F, OH, O-alkyl, NH₂ and N(alkyl)₂, in which alkyl is as described above and is preferably unsubstituted alkyl as described above.

For the purposes of the invention, heterocyclyl is preferably an unsaturated or preferably saturated cyclic radical, which preferably has 1 to 6 C atoms and 1 to 4 heteroatoms, preferably selected from N, S and O. Heterocyclyl is preferably a 5-, 6- or 7-membered ring as described above which is unsubstituted or substituted by 1 to 5 and in particular 1 to 3 substituents, where the substituents are preferably selected from Hal, A, OH, OA, NH₂, NO₂, CN, COOH, COOA, CONH₂, NHCOA, NHCONH₂, NHSO₂A, CHO, COA, SO₂NH₂, SO₂A, —CH₂—COOH, —OCH₂—COOH, ═O, ═S and ═N—R^{a} and in particular from Hal, in particular Cl and F, OH, O-alkyl, NH₂, N(alkyl)₂, ═O and ═S. Heterocyclyl is particularly preferably selected from 1-piperidyl, 4-piperidyl, 1-methyl piperidin-4-yl, 1-piperazyl, 1-(4-methyl)piperazyl, 4-methylpiperazin-1-ylamine, 1-(4-(2-hydroxyethyl))piperazyl, 4-morpholinyl, 1-pyrrolidinyl, 2-pyrrolidinyl, 3-pyrrolidinyl, 1-pyrazolidinyl 1-(2-methyl)pyrazolidinyl, 1-imidazolidinyl or 1-(3-methyl)imidazolidinyl, thiophen-2-yl, thiophen-3-yl, 2-oxazolyl, 4-oxazolyl, 5-oxazolyl, 2-thiazolyl, 4-thiazolyl and 5-thiazolyl, where, of the radicals mentioned above, the saturated radicals are particularly preferred. Particular preference is likewise given to the radicals mentioned above, in particular the saturated radicals mentioned above, which have 1, 2 or 3, preferably one or two, substituents selected from A, ═O, ═S, ═N—R^{a} and/or Hal. Heterocyclyl is very particularly preferably a saturated radical as defined above which is either unsubstituted or mono- or disubstituted by A and/or ═O.

A denotes alkyl, preferably as described above, and is particularly preferably unbranched (linear) or branched, and has 1, 2, 3, 4, 5, 6, 7, 8, 9 or 10 C atoms. A preferably denotes methyl, furthermore ethyl, propyl, isopropyl, butyl, isobutyl, sec-butyl or tert-butyl, furthermore also pentyl, 1-, 2- or 3-methylbutyl, 1,1-, 1,2- or 2,2-dimethylpropyl, 1-ethylpropyl, hexyl, 1-, 2-, 3- or 4-methylpentyl, 1,1-, 1,2-, 1,3-, 2,2-, 2,3- or 3,3-dimethylbutyl, 1- or 2-ethylbutyl, 1-ethyl-1-methylpropyl, 1-ethyl-2-methylpropyl, 1,1,2- or 1,2,2-trimethylpropyl, further preferably, for example, trifluoromethyl.

A very particularly preferably denotes alkyl having 1, 2, 3, 4, 5 or 6 C atoms, preferably methyl, ethyl, propyl, isopropyl, butyl, isobutyl, sec-butyl, tert-butyl, pentyl, hexyl, trifluoromethyl, pentafluoroethyl or 1,1,1-trifluoroethyl. A also denotes cycloalkyl. Cycloalkyl preferably denotes cyclopropyl, cyclobutyl, cyclopentyl, cyclohexyl or cycloheptyl, but in particular cyclopentyl.

Ar preferably stands for aryl radicals and heteroaryl radicals as described herein and in particular for aryl radicals as described herein.

Het preferably stands for heteroaryl radicals and heterocyclyl radicals as described herein and in particular for heterocyclyl radicals as described herein.

R¹, R², R³ and R⁴ are preferably in each case, independently of one another, selected from H, A, CF₃, OCF₃, OR^{a}, SA, S(O)₂A, S(O)A, CH₂CN, COOA, CONHA, Hal, SCF, CN and Het, preferably also from H, Cl, Br, F, t-butyl, —CH(CH₃)CH₂CH₃, isopropyl, ethyl and methyl. R¹, R², R³ and R⁴ are particularly preferably in each case, independently of one another, selected from H, t-butyl, isopropyl, ethyl, CF₃, methyl, Br, Cl, F, SCF₃, CH(CH₃)CH₂CH₃, n-propyl, OCH₃, SCH₃, n-butyl, CH₂CN and Het. In particular, R¹, R², R³ and R⁴ are in each case, independently of one another, selected from H, Cl, Br, F, t-butyl, isopropyl, ethyl or CF₃.

Preferably, at least one of the radicals R¹, R², R³ and R⁴ is different from H. Particularly preferably, one, two or three of the radicals R¹, R², R³ and R⁴ are different from H.

Preferably, at least one of the radicals R¹, R², R³ and R⁴, particularly preferably one, two or three of the radicals R¹, R², R³ and R⁴, is therefore in each case, independently of one another, selected from A, Ar, Het, OR^{a}, SR^{a}, OAr, SAr, N(R^{a})₂, NR^{a}Ar, Hal, NO₂, CN, (CH₂)ₘCOOR^{a}, (CH₂)ₘCOOAr, (CH₂)ₘCON(R^{a})₂, (CH₂)ₘCONHAr, COR^{a}, COAr, S(O)ₘA, S(O)ₘAr, NHCOA, NHCOAr, NHSO₂A, NHSO₂Ar and SO₂N(R^{a})₂.

Particularly preferably, at least one of the radicals R¹, R², R³ and R⁴, particularly preferably one, two or three of the radicals R¹, R², R³ and R⁴, is therefore in each case, independently of one another, selected from A, CF₃, OCF₃, OR^{a}, SA, S(O)₂A, S(O)A, CH₂CN, COOA, CONHA, Hal, SCF, CN and Het, preferably also from H, Cl, Br, F, t-butyl, —CH(CH₃)CH₂CH₃, isopropyl, ethyl and methyl, very particularly preferably selected from t-butyl, isopropyl, ethyl, CF₃, methyl, Br, Cl, F, SCF₃, CH(CH₃)CH₂CH₃, n-propyl, OCH₃, SCH₃, n-butyl, CH₂CN and Het, and in particular selected from Cl, Br, F, t-butyl, isopropyl, ethyl or CF₃.

R¹ and/or R⁴ preferably stands for H.

R² and/or R³ preferably stands for a radical other than H, preferably selected from the radicals other than H mentioned above.

R² and R³ are preferably, independently of one another, selected from H, Cl, F, CH₃, C(CH₃)₃, CF₃ and OCH₃, particularly preferably with the proviso that one or both of the radicals R², R³ are different from H.

R² and/or R³ preferably stands for Hal, A or OA, in particular Cl, F, CH₃, C(CH₃)₃, cyclopropyl, CF₃ or OCH₃.

In particularly preferred compounds according to the invention, one of the radicals R² and R³ has the meaning H and the other radical has the meaning Cl, F, CH₃, C(CH₃)₃, CF₃ or OCH₃. Particularly preferably, the radical R³ here has the meaning H and the radical R² has the meaning the meaning Cl, F, CH₃, C(CH₃)₃, CF₃ or OCH₃.

In further particularly preferred compounds according to the invention, both radicals R² and R³ are selected independently from Cl, F, CH₃, C(CH₃)₃, CF₃ and OCH₃. The radical R³ here is particularly preferably selected from F, Cl and CH₃, and the radical R² is particularly preferably selected independently from Cl, F, CH₃, C(CH₃)₃, CF₃ and OCH₃.

In further particularly preferred compounds according to the invention, R³ stands for H and R² stands for Cl or CF₃.

R^{a} preferably denotes H, A, Ar, Het, aralkyl or heteroaralkyl, particularly preferably H, A, Ar, Het or aralkyl 1, very particularly preferably H, A, Ar or Het and in particular H, A or Ar. If R^{a} stands for A, Ar, Het, aralkyl or heteroaralkyl, the said radicals may also be substituted. R^{a} then generally has 1 to 5 and preferably 1 to 3 substituents, preferably selected from Hal, A, OH, OA, NH₂, NO₂, CN, COOH, COOA, CONH₂, NHCOA, NHCONH₂, NHSO₂A, CHO, COA, SO₂NH₂, SO₂A, —CH₂—COOH and —OCH₂—COOH and in particular from Hal, in particular Cl and F, OH, O-alkyl, NH₂ and N(alkyl)₂, in which alkyl is as described above and is preferably unsubstituted alkyl as described above.

R⁵ preferably denotes H, A, Ar, Het, aralkyl or heteroaralkyl, particularly preferably A, Ar, Het, aralkyl or heteroaralkyl, very particularly preferably A, Ar, aralkyl or heteroaralkyl and in particular aralkyl or heteroaralkyl. In particular, R⁵ denotes benzyl, phenethyl, or phenylpropyl, or substituted derivatives thereof. If R⁵ stands for substituted benzyl, phenethyl, or phenylpropyl, it has 1 to 5 and preferably 1 to 3 substituents, preferably selected from Hal, A, OH, OA, NH₂, NO₂, CN, COOH, COOA, CONH₂, NHCOA, NHCONH₂, NHSO₂A, CHO, COA, SO₂NH₂, SO₂A, —CH₂—COOH and —OCH₂—COOH and in particular from Hal, in particular Cl and F, OH, O-alkyl, NH₂ and N(alkyl)₂, in which alkyl is as described above and is preferably unsubstituted alkyl as described above. R⁵ particularly preferably denotes substituted or unsubstituted benzyl and in particular unsubstituted benzyl.

R⁸ preferably denotes H, A, Ar, Het, aralkyl or heteroaralkyl, particularly preferably A, Ar, Het, aralkyl or heteroaralkyl, very particularly preferably A, Ar, aralkyl or heteroaralkyl and in particular A or Ar. If R⁸ stands for Ar, Ar is preferably unsubstituted or substituted phenyl. If R⁸ stands for substituted phenyl, it has 1 to 5 and preferably 1 to 3 substituents, preferably selected from Hal, A, OH, OA, NH₂, NO₂, CN, COOH, COOA, CONH₂, NHCOA, NHCONH₂, NHSO₂A, CHO, COA, SO₂NH₂, SO₂A, —CH₂—COOH and —OCH₂—COOH and in particular from Hal, in particular Cl and F, OH, O-alkyl, NH₂ and N(alkyl)₂, in which alkyl is as described above and is preferably unsubstituted alkyl as described above.

R⁶ preferably denotes H, A, Ar, aralkyl or heteroaralkyl, particularly H, A, or aralkyl. If R⁶ stands for A, A is preferably substituted or unsubstituted alkyl having 1 to 6 C atoms, and in particular methyl or ethyl. If R⁶ stands for Ar, it preferably stands for substituted or unsubstituted phenyl. If R⁶ stands for aralkyl, aralkyl is preferably selected from benzyl, phenethyl, phenylpropyl, phenylbutyl, benzoyl, 2-phenylacetyl and 3-phenylpropionyl or butyryl, and substituted derivatives thereof, in particular substituted derivatives in which the phenyl radical has 1, 2 or 3 substituents as defined herein.

R⁷ preferably denotes H, A, Ar, aralkyl or heteroaralkyl, particularly H, A, or aralkyl. If R⁶ stands for A, A is preferably substituted or unsubstituted alkyl having 1 to 6 C atoms, and in particular methyl or ethyl. If R⁶ stands for Ar, it preferably stands for substituted or unsubstituted phenyl. If R⁶ stands for aralkyl, aralkyl is preferably selected from benzyl, phenethyl, phenylpropyl, phenylbutyl, benzoyl, 2-phenylacetyl and 3-phenylpropionyl or butyryl, and substituted derivatives thereof, in particular substituted derivatives in which the phenyl radical has 1, 2 or 3 substituents as defined herein.

Alternatively, R⁶ and R⁷, together with the N atom to which they are bonded stand for a saturated or unsaturated 5-, 6- or 7-membered heterocycle, preferably form a saturated or unsaturated 5- or 6-membered heterocycle, which may optionally contain 1, 2 or 3 further heteroatoms, preferably 1 or 2 further heteroatoms, which are preferably selected from N, S and O and in particular from N and O. In this embodiment, NR⁶R⁷ preferably stands for 1-piperidyl, 1-piperazyl, 1-(4-methyl)piperazyl, 4-methylpiperazin-1-ylamine, 4-morpholinyl, 1-pyrrolidinyl, 1-pyrazolidinyl 1-(2-methyl)pyrazolidinyl, 1-imidazolidinyl or 1-(3-methyl)imidazolidinyl, 4-pyridyl, oxazolyl, thiazolyl, quinolinyl, isoquinolinyl, 2- or 4-pyridazyl, 2-, 4- or 5-pyrimidyl, 2- or 3-pyrazinyl.

Y¹ preferably stands for O or S and in particular for O.

Y² preferably stands for O or S and in particular for O.

Z¹ preferably stands for (CR⁹R¹⁰)ₙ or (CR⁹R¹⁰)ₚ—(C═Y²)—(CR¹¹R¹²)_{q}, in which n preferably stands for 2, 3 or 4, particularly preferably 2 or 3 and in particular for 2, and in which p preferably stands for 0, 1 or 2, particularly preferably 0 or 1 and in particular for 0, and in which q preferably stands for 0, 1 or 2, particularly preferably 0 or 1 and in particular for 0. In (CR⁹R¹⁰)ₙ, R⁹ and R¹⁰ are preferably, independently of one another, selected from H, A, OA, Ar, Het, aralkyl and heteroaralkyl, particularly preferably from H, A, Ar and aralkyl and in particular from H and A. In (CR⁹R¹⁰)ₚ—(C═Y²)—(CR¹¹R¹²)_{q}, R⁹, R¹⁰; R¹¹ and R¹² are preferably, independently of one another, selected from H, A, OA, Ar, Het, aralkyl and heteroaralkyl, particularly preferably from H, A, Ar and aralkyl and in particular from H and A.

In Z¹, each CR⁹R¹⁰ group in (CR⁹R¹⁰)ₙ is in each case, independently of one another, preferably selected from CR⁹R¹⁰, CHA, CAA and CH₂. In Z¹, (CR⁹R¹⁰)ₚ—(C═Y²)—(CR¹¹R¹²)_{q} is preferably selected from CR⁹R¹⁰—(C═Y²), (C═Y²)—CR¹¹R¹² and C═Y², and in particular from CR⁹R¹⁰—(C═O), (C═O)—CR¹¹R¹² and C═O.

The (CR⁹R¹⁰)ₙ group in Z¹ thus preferably stands for groups selected from selected from CR⁹R¹⁰CR⁹R¹⁰, CR⁹R¹⁰CH₂, CH₂CR⁹R¹⁰, CHACHA, CAACAA, CHACH₂, CH₂CHA and CH₂CH₂.

The (CR⁹R¹⁰)ₚ—(C═Y²)—(CR¹¹R¹²)_{q} group in Z¹ thus preferably stands for groups selected from CR⁹R¹⁰—(C═Y²), (C═Y²)—CR¹¹R¹² and C═Y², particularly preferably selected from CR⁹R¹⁰—(C═O), (C═O)—CR¹¹R¹² and C═O, very particularly preferably selected from CR⁹R¹⁰—(C═O) and (C═O)—CR¹¹R¹², and in particular selected from CHA-(C═O) and (C═O)—CHA.

Z¹ particularly preferably stands for CR⁹R¹⁰CH₂, CH₂CR⁹R¹⁰, CHACHA, CAACAA, CHACH₂, CH₂CHA, CR⁹R¹⁰—(C═O), (C═O)—CR¹¹R¹², CHA-(C═O) or (C═O)—CHA, very particularly preferably for and in particular for CHACH₂, CH₂CHA, CHA-(C═O) or (C═O)—CHA, and in particular for CHACH₂ or CHA(C═O).

Z² preferably stands for (CR⁹R¹⁰)ₙ or (CR⁹R¹⁰)ₚ—(C═Y²)—(CR¹¹R¹²)_{q}, in which n preferably stands for 1, 2, 3 or 4, particularly preferably 1, 2 or 3 and in particular for 2 or 3, in which p preferably stands for 0, 1 or 2 and particularly preferably for 0 or 1, and in which q preferably stands for 0, 1 or 2 and particularly preferably for 0 or 1. In (CR⁹R¹⁰)ₙ, R⁹ and R¹⁰ are preferably, independently of one another, selected from H, A, OA, Ar, Het, aralkyl and heteroaralkyl, particularly preferably from H, A, Ar and aralkyl and in particular from H and A. In (CR⁹R¹⁰)ₚ—(C═Y²)—(CR¹¹R¹²)_{q}, R⁹, R¹⁰; R¹¹ and R¹² are preferably, independently of one another, selected from H, A, OA, Ar, Het, aralkyl and heteroaralkyl, particularly preferably from H, A, Ar and aralkyl and in particular from H and A.

In Z², (CR⁹R¹⁰)ₙ is preferably selected from CR⁹R¹⁰, (CR⁹R¹⁰)₂, (CR⁹R¹⁰)₃ and (CR⁹R¹⁰)₄, in which R⁹ and R¹⁰ are as defined above/below and are preferably in each case, independently of one another, selected from H and A, and particularly preferably selected from CHA, (CHA)₂, (CHA)₃ and (CHA)₄, in which each A, independently of one another, is as defined herein. In Z², (CR⁹R¹⁰)ₙ is very particularly preferably selected from CH₂, (CH₂)₂, (CH₂)₃ and (CH₂)₄. In Z², (CR⁹R¹⁰)ₚ—(C═Y²)—(CR¹¹R¹²)_{q} is preferably selected from CR⁹R¹⁰—(C═Y²)—CR¹¹R¹², CR⁹R¹⁰—(C═Y²), (C═Y²)—CR¹¹R¹², CR⁹R¹⁰CR⁹R¹⁰—(C═Y²), (C═Y²)—CR¹¹R¹²CR¹¹R¹² and C═Y², and in particular from CR⁹R¹⁰—(C═O), (C═O)—CR¹¹R¹² and C═O.

Z² is particularly preferably selected from CR⁹R¹⁰, (CR⁹R¹⁰)₂, (CR⁹R¹⁰)₃ and (CR⁹R¹⁰)₄, in which R⁹ and R¹⁰ are as defined above/below and are preferably in each case, independently of one another, selected from H and A, and in particular from CH₂, (CH₂)₂, (CH₂)₃ and (CH₂)₄.

Z³ preferably stands for (CR⁹R¹⁰)ₙ or (CR⁹R¹⁰)ₚ—(C═Y²)—(CR¹¹R¹²)_{q}, in which n preferably stands for 1, 2 or 3, particularly preferably 1 or 2 in particular for 1, in which p preferably stands for 0, 1 or 2, particularly preferably 0 or 1 and in particular for 0, and in which q preferably stands for 0, 1 or 2, particularly preferably 0 or 1 and in particular for 0. In (CR⁹R¹⁰)ₙ, R⁹ and R¹⁰ are preferably, independently of one another, selected from H, A, OA, Ar, Het, aralkyl and heteroaralkyl, particularly preferably from H, A, Ar and aralkyl and in particular from H and A. In (CR⁹R¹⁰)ₚ—(C═Y²)—(CR¹¹R¹²)_{q}, R⁹, R¹⁰; R¹¹ and R¹² are preferably, independently of one another, selected from H, A, OA, Ar, Het, aralkyl and heteroaralkyl, particularly preferably from H, A, Ar and aralkyl and in particular from H and A.

In Z³, (CR⁹R¹⁰)ₙ is preferably selected from CR⁹R¹⁰, CHA, CAA and CH₂. In Z³, (CR⁹R¹⁰)ₚ—(C═Y²)—(CR¹¹R¹²)_{q} is preferably selected from CR⁹R¹⁰—(C═Y²), (C═Y²)—CR¹¹R¹² and C═Y², and in particular from CR⁹R¹⁰—(C═O), (C═O)—CR¹¹R¹² and C═O.

Z³ is particularly preferably selected from CH₂, (CH₂)₂, (CH₂)₃ and (CH₂)₄, C═Y², C═O and C═S, very particularly preferably from CH₂, C═Y², C═O and C═S.

In preferred compounds according to the invention, Z³ may also be absent, i.e. the radical R⁸ is bonded directly to the N atom. In these cases, the -(Z¹-N(-Z³-R⁸)-Z²)ₖ- group stands for the -(Z¹-N(—R⁸)-Z²)ₖ- group.

R⁸-Z³ particularly preferably stands for H, substituted or preferably unsubstituted methyl, ethyl, propyl or butyl, substituted or preferably unsubstituted benzyl, phenethyl, phenylpropyl, phenylbutyl, benzoyl, 2-phenylacetyl, 3-phenylpropionyl or 4-phenylbutyryl. If R⁸-Z³ stands for substituted methyl, ethyl, propyl or butyl, it has 1 to 5 and preferably 1 to 3 substituents, preferably selected from Hal, A, OH, OA, NH₂, NO₂, CN, COOH, COOA, CONH₂, NHCOA, NHCONH₂, NHSO₂A, CHO, COA, SO₂NH₂, SO₂A, —CH₂—COOH or —OCH₂—COOH and particularly preferably from Hal, in particular Cl and F, OH, O-alkyl, NH₂ and N(alkyl)₂, in which alkyl is as described above and is preferably unsubstituted alkyl as described above. If R⁸-Z³ stands for substituted benzyl, phenethyl, phenylpropyl, phenylbutyl, benzoyl, 2-phenylacetyl, 3-phenylpropionyl or 4-phenylbutyryl, it has 1 to 5 and preferably 1 to 3 substituents, preferably selected from Hal, A, OH, OA, NH₂, NO₂, CN, COOH, COOA, CONH₂, NHCOA, NHCONH₂, NHSO₂A, CHO, COA, SO₂NH₂, SO₂A, —CH₂—COOH or —OCH₂—COOH and particularly preferably from Hal, in particular Cl and F, OH, O-alkyl, NH₂ and N(alkyl)₂, in which alkyl is as described above and is preferably unsubstituted alkyl as described above. The substituents here are preferably arranged on the phenyl ring.

Hal preferably denotes F, Cl or Br, but also 1, particularly preferably F or Cl.

Throughout the invention, all radicals which occur more than once may be identical or different, i.e. are independent of one another.

The compounds of the formula I can have one or more chiral centres and therefore occur in various stereoisomeric forms. The formula I encompasses all these forms.

Particularly preferred compounds of the formula I are the compounds of the sub-formulae IA to IT:

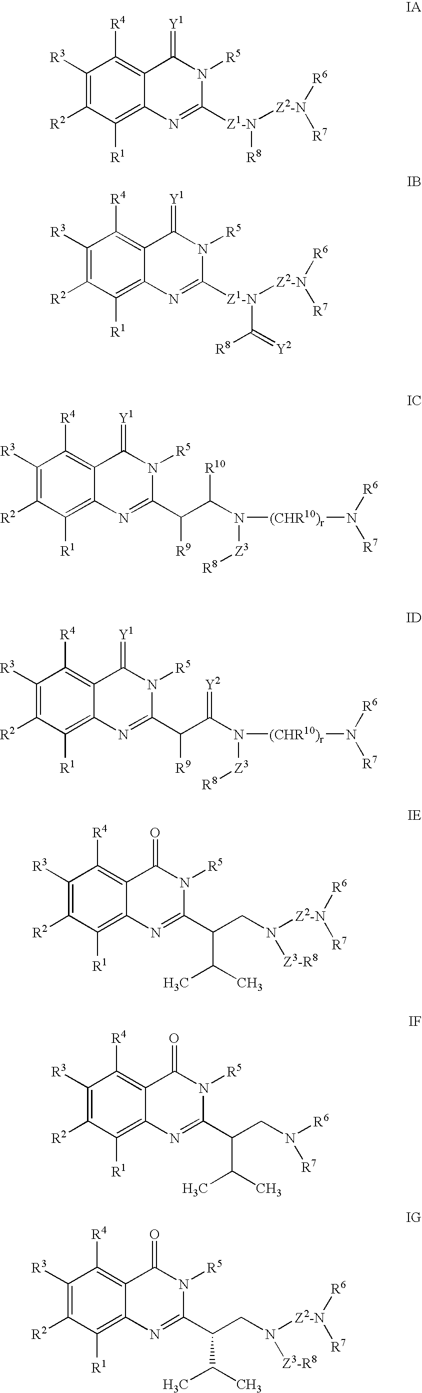

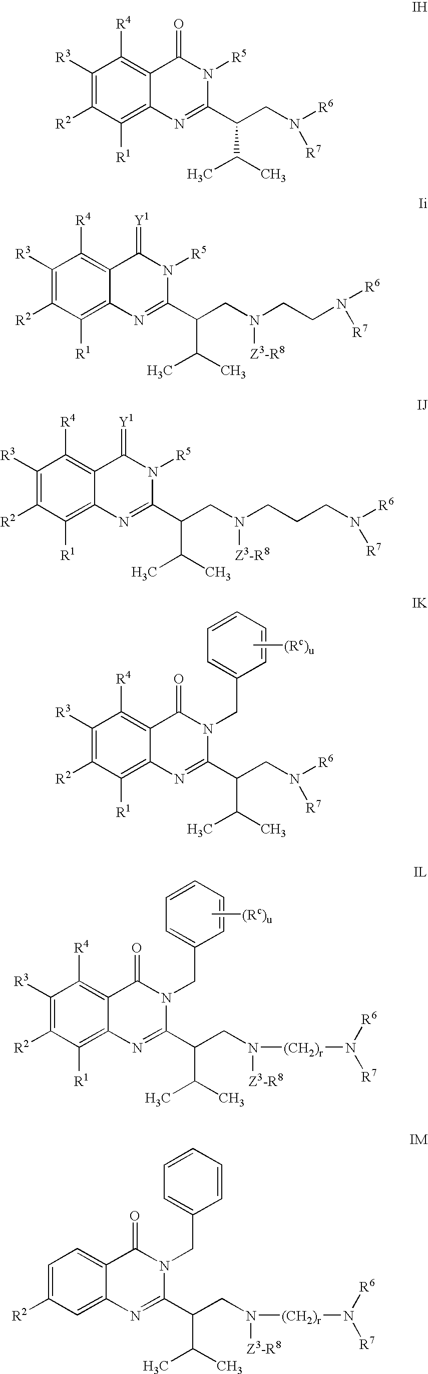

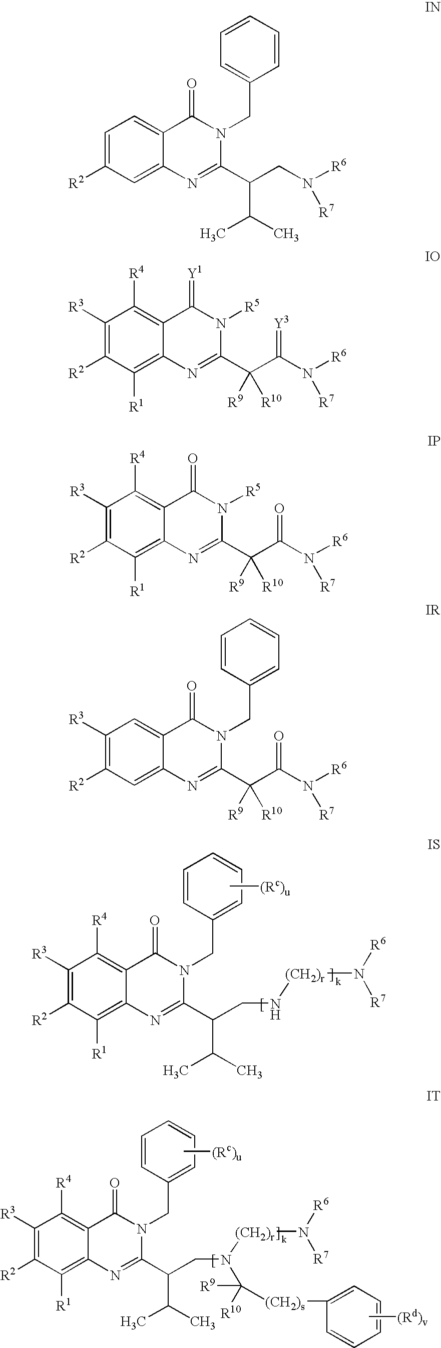

in which
R¹, R², R³, R⁴, R⁵, R⁶, R⁷, R⁸, R⁹, R¹⁰, Y¹, Y², Z¹ and Z³ have the meanings indicated herein,
r stands for 1, 2, 3 or 4, preferably for 1, 2, or 3 and in particular for 2 or 3,
s and t, independently of one another, stands for 0, 1 or 2 and in particular for 0 or 1,
Y³ stands for O, S or NR^{a} and in particular for O or S,
R¹⁹ and R²⁰, independently of one another, are selected from the meanings indicated for R⁹ and R¹⁰ or together stand for ═O, ═S or ═NH,
R^{c} and R^{d}, independently of one another, are selected from the meanings indicated for R¹ to R⁴, and
u and v, independently of one another, stands for 0, 1, 2 or 3 and in particular for 0, 1 or 2.
   R^{c} is preferably selected from A, CF₃, OCF₃, OR^{a}, SA, S(O)₂A, S(O)A, CH₂CN, COOA, CONHA, Hal, SCF, CN and Het, particularly preferably also from H, Cl, Br, F, t-butyl, —CH(CH₃)CH₂CH₃, isopropyl, ethyl and methyl, very particularly preferably selected from t-butyl, isopropyl, ethyl, CF₃, methyl, Br, Cl, F, SCF₃, CH(CH₃)CH₂CH₃, n-propyl, OCH₃, SCH₃, n-butyl, CH₂CN and Het, and in particular selected from Cl, Br, F, t-butyl, isopropyl, ethyl and CF₃.
   R^{d} is preferably selected from A, CF₃, OCF₃, OR^{a}, SA, S(O)₂A, S(O)A, CH₂CN, COOA, CONHA, Hal, SCF, CN and Het, particularly preferably also from H, Cl, Br, F, t-butyl, —CH(CH₃)CH₂CH₃, isopropyl, ethyl and methyl, very particularly preferably selected from t-butyl, isopropyl, ethyl, CF₃, methyl, Br, Cl, F, SCF₃, CH(CH₃)CH₂CH₃, n-propyl, OCH₃, SCH₃, n-butyl, CH₂CN and Het, and in particular selected from Cl, Br, F, t-butyl, isopropyl, ethyl and CF₃.

Particular preference is given to compounds according to the invention, i.e. compounds of the formula I and preferably of the formula I′, I″, I′″, Iα, Iβ, IA, IB, IC, ID, IE, IF, IG, IH, Ii, IJ, IK, IL, IM, IN, IO, IP, IR, IS and/or IT, which combine one or preferably more of the preferred embodiments described below:

A preferred embodiment relates to compounds according to the invention in which
R² denotes A, CF₃, OCF₃, SA, SCN, CH₂CN, —OCOA, Hal, SCF₃, t-butyl, —CH(CH₃)CH₂CH₃, isopropyl, ethyl or methyl.

A further preferred embodiment relates to compounds according to the invention in which
R³ denotes H, A, CF₃, OCF₃, SA, SCN, CH₂CN, —OCOA, Hal, SCF₃, t-butyl, —CH(CH₃)CH₂CH₃, isopropyl, ethyl or methyl.

A further preferred embodiment relates to compounds according to the invention in which
R¹ and R⁴, independently of one another, either denote H or are selected from A, CF₃, OCF₃, OR^{a}, SA, S(O)₂A, S(O)A, CH₂CN, COOA, CONHA, Hal, SCF, CN and Het.

A further preferred embodiment relates to compounds according to the invention in which
R³ and R⁴, independently of one another, are selected from H and Cl.

A further preferred embodiment relates to compounds according to the invention in which
R⁵ is selected from Ar, aralkyl and heteroaralkyl, preferably from aralkyl and heteroaralkyl and in particular from benzyl and phenethyl;
R⁶, R⁷, independently of one another, is selected from H, A, Ar and aralkyl, preferably from H and A and in particular from H, methyl and ethyl; and
R⁸ is selected from H, A, Ar and Het, preferably from A, Ar and Het, very particularly preferably from Ar and Het and in particular from phenyl and pyridyl.

A further preferred embodiment relates to compounds according to the invention in which
R⁵ denotes unsubstituted or substituted benzyl, and
R⁸ denotes unsubstituted or substituted phenyl.

A further preferred embodiment relates to compounds according to the invention in which R⁶ and R⁷, together with the N atom to which they are bonded, form a saturated or unsaturated 5-, 6- or 7-membered heterocycle, which may optionally contain 1, 2 or 3 further heteroatoms selected from N, S and O and which is particularly preferably selected from 1-piperidyl, 4-piperidyl, 1-methyl piperidin-4-yl, 1-piperazyl, 1-(4-methyl)piperazyl, 4-methylpiperazin-1-ylamine, 1-(4-(2-hydroxyethyl))piperazyl, 4-morpholinyl and 1-pyrrolidinyl.

Accordingly, the invention relates, in particular, to the compounds according to the invention, in particular compounds of the formula I and/or the sub-formulae thereof, in which at least one of the said radicals has one of the preferred meanings indicated above and/or which combine one or more of the preferred embodiments described herein.

The compounds according to the invention are particularly preferably selected from the following compounds:

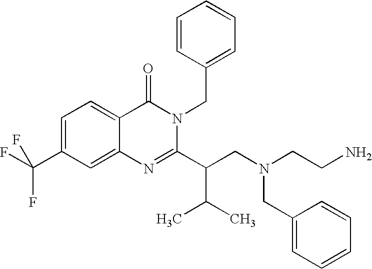

2-(1-{[(2-aminoethyl)benzylamino]methyl}-2-methylpropyl)-3-benzyl-7-trifluoromethyl-3H-quinazolin-4-one (MW=522.61; Rt=2.59);

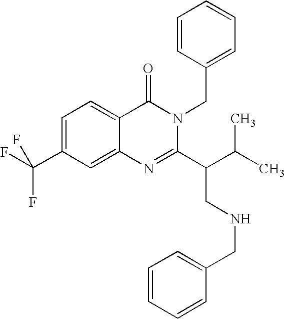

3-benzyl-2-[1-(benzylaminomethyl)-2-methylpropyl]-7-trifluoromethyl-3H-quinazolin-4-one (MW=479.54; Rt=2.6);

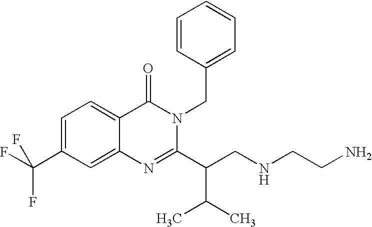

2-{1-[(2-aminoethylamino)methyl]-2-methylpropyl}-3-benzyl-7-trifluoromethyl-3H-quinazolin-4-one (MW=432.49; Rt=1.85);

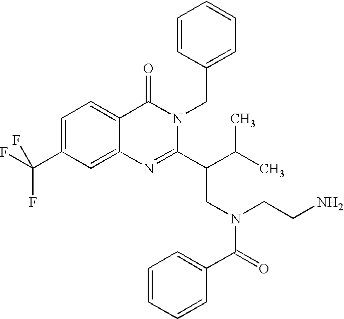

N-(2-aminoethyl)-N-[2-(3-benzyl-4-oxo-7-trifluoromethyl-3,4-dihydroquinazolin-2-yl)-3-methylbutyl]benzamide (MW=536.60; Rt=2.37);

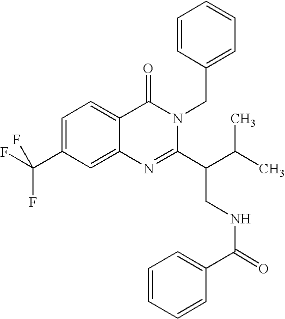

N-[2-(3-benzyl-4-oxo-7-trifluoromethyl-3,4-dihydroquinazolin-2-yl)-3-methylbutyl]benzamide (MW=493.53; Rt=3.31);

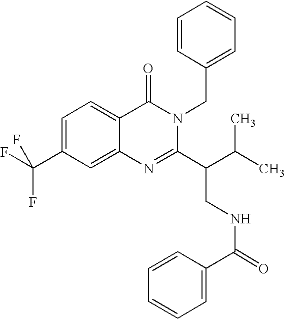

N-[2-(3-benzyl-4-oxo-7-trifluoromethyl-3,4-dihydroquinazolin-2-yl)-3-methylbutyl]-2-phenylacetamide (MW=507.55; Rt=3.29);

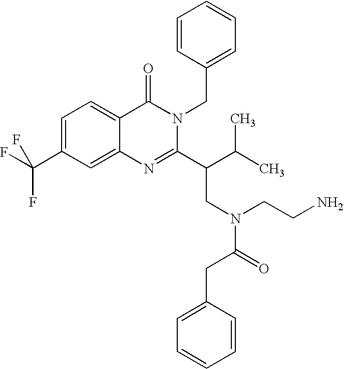

N-(2-aminoethyl)-N-[2-(3-benzyl-4-oxo-7-trifluoromethyl-3,4-dihydroquinazolin-2-yl)-3-methylbutyl]-2-phenylacetamide (MW=550.52; Rt=2.43);

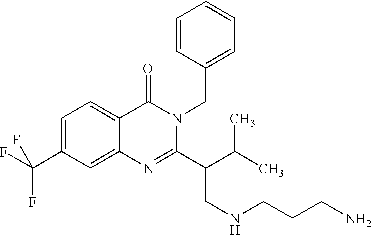

2-{1-[(3-aminopropylamino)methyl]-2-methylpropyl}-3-benzyl-7-trifluoromethyl-3H-quinazolin-4-one (MW=446.51; Rt=1.85);

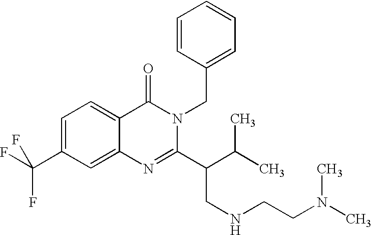

3-benzyl-2-{1-[(2-dimethylaminoethylamino)methyl]-2-methylpropyl}-7-trifluoromethyl-3H-quinazolin-4-one (MW=460.54; Rt=1.95);

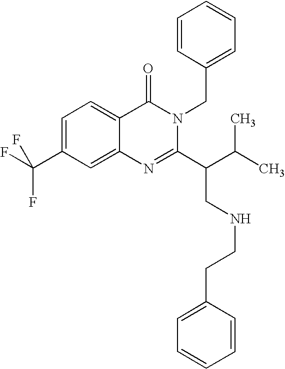

3-benzyl-2-[2-methyl-1-(phenethylaminomethyl)propyl]-7-trifluoromethyl-3H-quinazolin-4-one (MW=493.57; Rt=2.65);

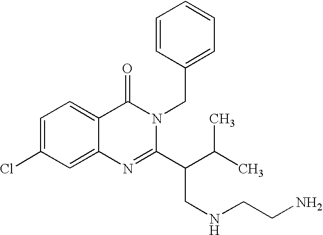

2-{1-[(2-aminoethylamino)methyl]-2-methylpropyl}-3-benzyl-7-chloro-3H-quinazolin-4-one (MW=398.94; Rt=1.65);

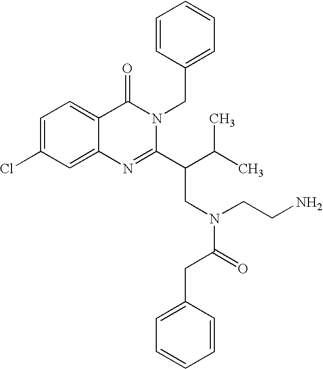

N-(2-aminoethyl)-N-[2-(3-benzyl-7-chloro-4-oxo-3,4-dihydroquinazolin-2-yl)-3-methylbutyl]-2-phenylacetamide (MW=517.07; Rt=2.25);

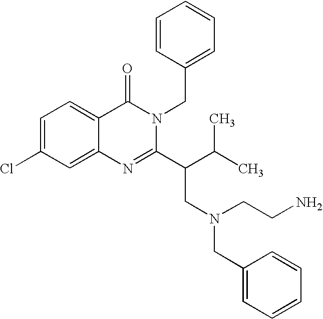

2-(1-{[(2-aminoethyl)benzylamino]methyl}-2-methylpropyl)-3-benzyl-7-chloro-3H-quinazolin-4-one (MW=489.06; Rt=2.42);

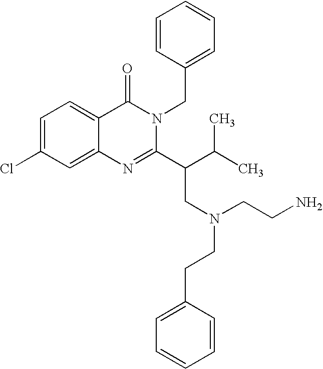

2-(1-{[(2-aminoethyl)phenethylamino]methyl}-2-methylpropyl)-3-benzyl-7-chloro-3H-quinazolin-4-one (MW=503.09; Rt=2.39);

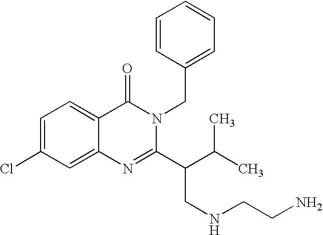

2-{1-[(3-aminopropylamino)methyl]-2-methylpropyl}-3-benzyl-7-chloro-3H-quinazolin-4-one (MW=412.96; Rt=1.65);

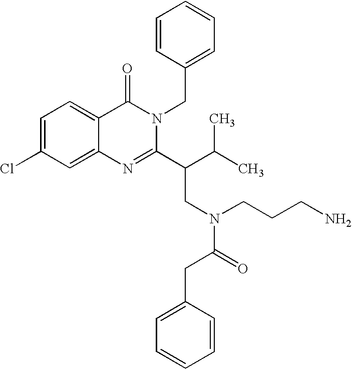

N-(3-aminopropyl)-N-[2-(3-benzyl-7-chloro-4-oxo-3,4-dihydroquinazolin-2-yl)-3-methylbutyl]-2-phenylacetamide (MW=531.10; Rt=2.29);

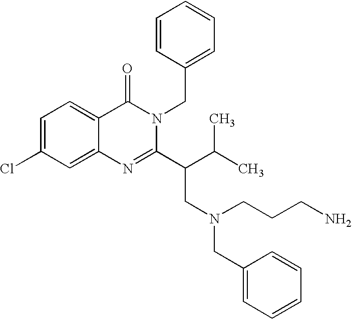

2-(1-{[(3-aminopropyl)benzylamino]methyl}-2-methylpropyl)-3-benzyl-7-chloro-3H-quinazolin-4-one (MW=503.09; Rt=1.92);

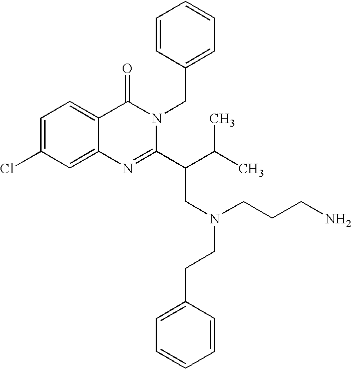

2-(1-{[(3-aminopropyl)phenethylamino]methyl}-2-methylpropyl)-3-benzyl-7-chloro-3H-quinazolin-4-one (MW=517.11; Rt=1.95);

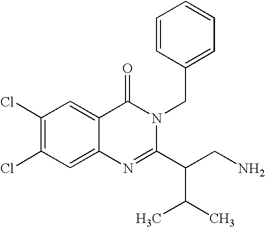

2-(1-aminomethyl-2-methylpropyl)-3-benzyl-6,7-dichloro-3H-quinazolin-4-one (MW=390.32; Rt=2.31);

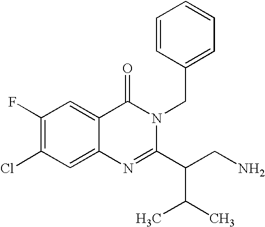

2-(1-aminomethyl-2-methylpropyl)-3-benzyl-7-chloro-6-fluoro-3H-quinazolin-4-one (MW=373.86; Rt=2.17);

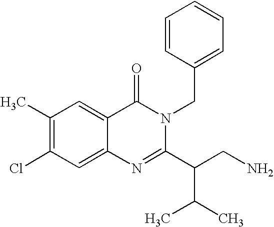

2-(1-aminomethyl-2-methylpropyl)-3-benzyl-7-chloro-6-methyl-3H-quinazolin-4-one (MW=369.89; Rt=2.22);

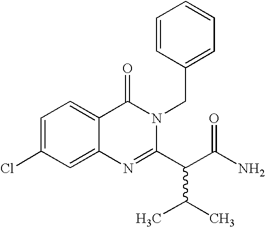

2-(3-benzyl-7-chloro-4-oxo-3,4-dihydroquinazolin-2-yl)-3-methylbutyramide;

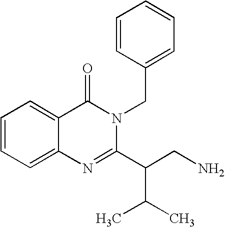

2-(1-aminomethyl-2-methylpropyl)-3-benzyl-3H-quinazolin-4-one (MW=321.42; Rt=1.9);

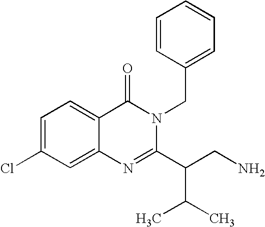

2-(1-aminomethyl-2-methylpropyl)-3-benzyl-7-chloro-3H-quinazolin-4-one (MW=355.87; Rt=2.13);

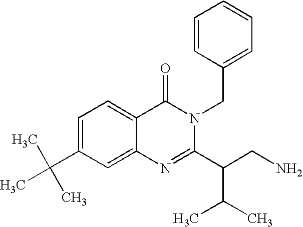

2-(1-aminomethyl-2-methylpropyl)-3-benzyl-7-tert-butyl-3H-quinazolin-4-one (MW=377.53; Rt=2.33);

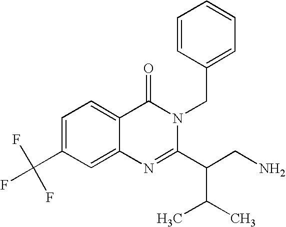

2-(1-aminomethyl-2-methylpropyl)-3-benzyl-7-trifluoromethyl-3H-quinazolin-4-one (MW=389.42; Rt=2.21);
   and the pharmaceutical tolerated derivatives, solvates, salts and stereoisomers thereof, including mixtures of the forms in all ratios, and preferably the salts and/or solvates thereof, and in particular the physiologically tolerated salts and/or solvates thereof.

Particular preference is furthermore given to the compounds according to the invention selected from the following compounds:

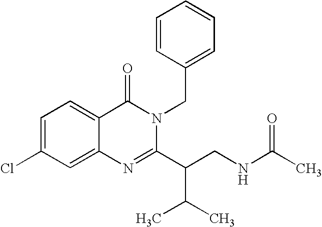

N-[2-(3-benzyl-7-chloro-4-oxo-3,4-dihydroquinazolin-2-yl)-3-methyl butyl]-acetamide;

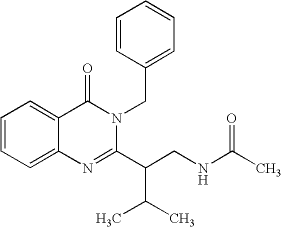

N-[2-(3-benzyl-4-oxo-3,4-dihydroquinazolin-2-yl)-3-methylbutyl]acetamide;

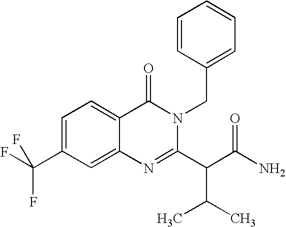

2-(3-benzyl-4-oxo-7-trifluoromethyl-3,4-dihydroquinazolin-2-yl)-3-methylbutyramide;

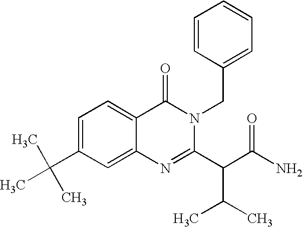

2-(3-benzyl-7-tert-butyl-4-oxo-3,4-dihydroquinazolin-2-yl)-3-methylbutyramide;

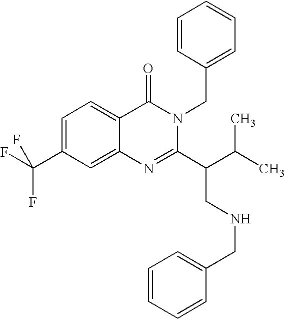

3-benzyl-2-[1-(benzylaminomethyl)-2-methylpropyl]-7-trifluoromethyl-3H-quinazolin-4-one;
   and the pharmaceutical tolerated derivatives, solvates, salts and stereoisomers thereof, including mixtures of the forms in all ratios, and preferably the salts and/or solvates thereof, and in particular the physiologically tolerated salts and/or solvates thereof.

The compounds according to the invention may, depending on the choice of the substituents and radicals described above, have one or more chiral centres, in particular one or more chiral carbon atoms. If a compound according to the invention of defined composition has one or more chiral centres, this compound of defined composition may exist in different stereoisomers. The present invention relates to all possible such stereoisomers of compounds according to the invention, which may be both in the form of individual stereochemically uniform compounds and also in the form of mixtures of two or more stereochemically uniform compounds. In the case of mixtures of two or more stereoisomers, the individual stereoisomers may be present in different or equal proportions. In the case of mixtures of two stereoisomers which are present in equal proportions and represent optical antipodes, the term racemic mixtures is used. The present invention likewise relates to racemic mixtures of compounds of the formula I.

The use of a hatched wedge instead of a line to represent a bond in a structural formula preferably indicates a three-dimensional indication of the structure of the corresponding compound or the corresponding chiral centre in this formula and denotes (in agreement with the corresponding general rules for the representation of stereoisomers) that the corresponding radical arranged on the broader side of the wedge is arranged behind the plane of the paper; see, for example, the representation of substructures IG and IH of the compounds according to the invention.

The compounds of the formula I and also the starting materials for their preparation are, in addition, prepared by methods known per se, as described in the literature (for example in the standard works, such as Houben-Weyl, Methoden der organischen Chemie [Methods of Organic Chemistry], Georg-Thieme-Verlag, Stuttgart), to be precise under reaction conditions which are known and suitable for the said reactions. Use may also be made here of variants known per se which are not mentioned here in greater detail.

If desired, the starting materials may also be formed in situ so that they are not isolated from the reaction mixture, but instead are immediately converted further into the compounds of the formula I.

The process steps of the process according to the invention are generally carried out in a solvent which is inert under the respective reaction conditions as mentioned, for example, herein, such as, for example, acetonitrile, preferably in the presence of a base, such as, for example, amines, preferably tertiary amines, hydroxides, in particular alkali metal hydroxides, such as KOH, or NaOH or in particular alkali metal or alkaline-earth metal carbonates, such as Na₂CO₃ and K₂CO₃. Depending on the conditions used, the reaction time is between a few minutes and 14 days, the reaction temperature is between about 0° and 180°, normally between 0° and 100°, particularly preferably between 15° C. and 60° C., very particularly preferably between 15° C. and 35° C., such as, for example, about 20° C., about 25° C. or about 45 degrees Celsius.

Suitable inert solvents are, for example, hydrocarbons, such as hexane, petroleum ether, benzene, toluene or xylene; chlorinated hydrocarbons, such as trichloroethylene, 1,2-dichloroethane, carbon tetrachloride, chloroform or dichloromethane; nitriles, such as acetonitrile; carbon disulfide; carboxylic acids, such as formic acid or acetic acid; nitro compounds, such as nitromethane or nitrobenzene, or mixtures of the said solvents.

If desired, a functionally modified amino and/or hydroxyl group in a compound of the formula I can be liberated by solvolysis or hydrogenolysis by conventional methods. This can be carried out, for example, using NaOH or KOH in water, water/THF or water/dioxane at temperatures between 0 and 100°.

The reduction of an ester to the aldehyde or alcohol or the reduction of a nitrile to the aldehyde or amine is carried out by methods as are known to the person skilled in the art and are described in standard works of organic chemistry.

The said compounds according to the invention can be used in their final non-salt form. On the other hand, the present invention also relates to the use of these compounds in the form of their pharmaceutically acceptable salts, which can be derived from various organic and inorganic acids and bases by procedures known in the art. Pharmaceutically acceptable salt forms of the compounds of the formula I are for the most part prepared by conventional methods. If the compound of the formula I contains a carboxyl group, one of its suitable salts can be formed by reacting the compound with a suitable base to give the corresponding base-addition salt. Such bases are, for example, alkali metal hydroxides, including potassium hydroxide, sodium hydroxide and lithium hydroxide; alkaline earth metal hydroxides, such as barium hydroxide and calcium hydroxide; alkali metal alkoxides, for example potassium ethoxide and sodium propoxide; and various organic bases, such as piperidine, diethanolamine and N-methylglutamine. The aluminium salts of the compounds of the formula I are likewise included. In the case of certain compounds of the formula I, acid-addition salts can be formed by treating these compounds with pharmaceutically acceptable organic and inorganic acids, for example hydrogen halides, such as hydrogen chloride, hydrogen bromide or hydrogen iodide, other mineral acids and corresponding salts thereof, such as sulfate, nitrate or phosphate and the like, and alkyl- and monoarylsulfonates, such as ethanesulfonate, toluenesulfonate and benzenesulfonate, and other organic acids and corresponding salts thereof, such as acetate, trifluoroacetate, tartrate, maleate, succinate, citrate, benzoate, salicylate, ascorbate and the like. Accordingly, pharmaceutically acceptable acid-addition salts of the compounds of the formula I include the following: acetate, adipate, alginate, arginate, aspartate, benzoate, benzenesulfonate (besylate), bisulfate, bisulfite, bromide, butyrate, camphorate, camphorsulfonate, caprylate, chloride, chlorobenzoate, citrate, cyclopentanepropionate, digluconate, dihydrogenphosphate, dinitrobenzoate, dodecylsulfate, ethanesulfonate, fumarate, galacterate (from mucic acid), galacturonate, glucoheptanoate, gluconate, glutamate, glycerophosphate, hemisuccinate, hemisulfate, heptanoate, hexanoate, hippurate, hydrochloride, hydrobromide, hydroiodide, 2-hydroxyethanesulfonate, iodide, isethionate, isobutyrate, lactate, lactobionate, malate, maleate, malonate, mandelate, metaphosphate, methanesulfonate, methylbenzoate, monohydrogenphosphate, 2-naphthalenesulfonate, nicotinate, nitrate, oxalate, oleate, palmate, pectinate, persulfate, phenylacetate, 3-phenylpropionate, phosphate, phosphonate, phthalate, but this does not represent a restriction.

Furthermore, the base salts of the compounds according to the invention include aluminium, ammonium, calcium, copper, iron(III), iron(II), lithium, magnesium, manganese(III), manganese(II), potassium, sodium and zinc salts, but this is not intended to represent a restriction. Of the above-mentioned salts, preference is given to ammonium; the alkali metal salts sodium and potassium, and the alkaline earth metal salts calcium and magnesium. Salts of the compounds of the formula I which are derived from pharmaceutically acceptable organic non-toxic bases include salts of primary, secondary and tertiary amines, substituted amines, also including naturally occurring substituted amines, cyclic amines, and basic ion exchanger resins, for example arginine, betaine, caffeine, chloroprocaine, choline, N,N′-dibenzylethylenediamine (benzathine), dicyclohexylamine, diethanolamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lidocaine, lysine, meglumine, N-methyl-D-glucamine, morpholine, piperazine, piperidine, polyamine resins, procaine, purines, theobromine, triethanolamine, triethylamine, trimethylamine, tripropylamine and tris(hydroxymethyl)methylamine (tromethamine), but this is not intended to represent a restriction.

Compounds of the present invention which contain basic nitrogen-containing groups can be quaternised using agents such as (C₁-C₄)alkyl halides, for example methyl, ethyl, isopropyl and tert-butyl chloride, bromide and iodide; di(C₁-C₄)alkyl sulfates, for example dimethyl, diethyl and diamyl sulfate; (C₁₀-C₁₈)alkyl halides, for example decyl, dodecyl, lauryl, myristyl and stearyl chloride, bromide and iodide; and aryl(C₁-C₄)alkyl halides, for example benzyl chloride and phenethyl bromide. Both water- and oil-soluble compounds according to the invention can be prepared using such salts.

The above-mentioned pharmaceutical salts which are preferred include acetate, trifluoroacetate, besylate, citrate, fumarate, gluconate, hemisuccinate, hippurate, hydrochloride, hydrobromide, isethionate, mandelate, meglumine, nitrate, oleate, phosphonate, pivalate, sodium phosphate, stearate, sulfate, sulfosalicylate, tartrate, thiomalate, tosylate and tromethamine, but this is not intended to represent a restriction.

The acid-addition salts of basic compounds of the formula I are prepared by bringing the free base form into contact with a sufficient amount of the desired acid, causing the formation of the salt in a conventional manner. The free base can be regenerated by bringing the salt form into contact with a base and isolating the free base in a conventional manner. The free base forms differ in a certain respect from the corresponding salt forms thereof with respect to certain physical properties, such as solubility in polar solvents; for the purposes of the invention, however, the salts otherwise correspond to the respective free base forms thereof.

As mentioned, the pharmaceutically acceptable base-addition salts of the compounds of the formula I are formed with metals or amines, such as alkali metals and alkaline earth metals or organic amines. Preferred metals are sodium, potassium, magnesium and calcium. Preferred organic amines are N,N′-dibenzylethylenediamine, chloroprocaine, choline, diethanolamine, ethylenediamine, N-methyl-D-glucamine and procaine.

The base-addition salts of acidic compounds according to the invention are prepared by bringing the free acid form into contact with a sufficient amount of the desired base, causing the formation of the salt in a conventional manner. The free acid can be regenerated by bringing the salt form into contact with an acid and isolating the free acid in a conventional manner. The free acid forms differ in a certain respect from the corresponding salt forms thereof with respect to certain physical properties, such as solubility in polar solvents; for the purposes of the invention, however, the salts otherwise correspond to the respective free acid forms thereof.

If a compound according to the invention contains more than one group which is capable of forming pharmaceutically acceptable salts of this type, the invention also encompasses multiple salts. Typical multiple salt forms include, for example, bitartrate, diacetate, difumarate, dimeglumine, diphosphate, disodium and trihydrochloride, but this is not intended to represent a restriction.

With regard to that stated above, it can be seen that the term “pharmaceutically acceptable salt” in the present connection is taken to mean an active ingredient which comprises a compound of the formula I in the form of one of its salts, in particular if this salt form imparts improved pharmacokinetic properties on the active ingredient compared with the free form of the active ingredient or any other salt form of the active ingredient used earlier. The pharmaceutically acceptable salt form of the active ingredient can also provide this active ingredient for the first time with a desired pharmacokinetic property which it did not have earlier and can even have a positive influence on the pharmacodynamics of this active ingredient with respect to its therapeutic efficacy in the body.

The invention furthermore relates to medicaments comprising at least one compound of the formula I and/or pharmaceutically usable derivatives, solvates and stereoisomers thereof, including mixtures thereof in all ratios, and optionally excipients and/or adjuvants.

Pharmaceutical formulations can be administered in the form of dosage units which comprise a predetermined amount of active ingredient per dosage unit. Such a unit can comprise, for example, 0.5 mg to 1 g, preferably 1 mg to 700 mg, particularly preferably 5 mg to 100 mg, of a compound according to the invention, depending on the condition treated, the method of administration and the age, weight and condition of the patient, or pharmaceutical formulations can be administered in the form of dosage units which comprise a predetermined amount of active ingredient per dosage unit. Preferred dosage unit formulations are those which comprise a daily dose or part-dose, as indicated above, or a corresponding fraction thereof of an active ingredient. Furthermore, pharmaceutical formulations of this type can be prepared using a process which is generally known in the pharmaceutical art.

Pharmaceutical formulations can be adapted for administration via any desired suitable method, for example by oral (including buccal or sublingual), rectal, nasal, topical (including buccal, sublingual or transdermal), vaginal or parenteral (including subcutaneous, intramuscular, intravenous or intradermal) methods. Such formulations can be prepared using all processes known in the pharmaceutical art by, for example, combining the active ingredient with the excipient(s) or adjuvant(s).

Pharmaceutical formulations adapted for oral administration can be administered as separate units, such as, for example, capsules or tablets; powders or granules; solutions or suspensions in aqueous or non-aqueous liquids; edible foams or foam foods; or oil-in-water liquid emulsions or water-in-oil liquid emulsions.

Thus, for example, in the case of oral administration in the form of a tablet or capsule, the active-ingredient component can be combined with an oral, non-toxic and pharmaceutically acceptable inert excipient, such as, for example, ethanol, glycerol, water and the like. Powders are prepared by comminuting the compound to a suitable fine size and mixing it with a pharmaceutical excipient comminuted in a similar manner, such as, for example, an edible carbohydrate, such as, for example, starch or mannitol. A flavour, preservative, dispersant and dye may likewise be present.

Capsules are produced by preparing a powder mixture as described above and filling shaped gelatine shells therewith. Glidants and lubricants, such as, for example, highly disperse silicic acid, talc, magnesium stearate, calcium stearate or polyethylene glycol in solid form, can be added to the powder mixture before the filling operation. A disintegrant or solubiliser, such as, for example, agar-agar, calcium carbonate or sodium carbonate, may likewise be added in order to improve the availability of the medicament after the capsule has been taken.

In addition, if desired or necessary, suitable binders, lubricants and disintegrants as well as dyes can likewise be incorporated into the mixture. Suitable binders include starch, gelatine, natural sugars, such as, for example, glucose or beta-lactose, sweeteners made from maize, natural and synthetic rubber, such as, for example, acacia, tragacanth or sodium alginate, carboxymethylcellulose, polyethylene glycol, waxes, and the like. The lubricants used in these dosage forms include sodium oleate, sodium stearate, magnesium stearate, sodium benzoate, sodium acetate, sodium chloride and the like. The disintegrants include, without being restricted thereto, starch, methylcellulose, agar, bentonite, xanthan gum and the like. The tablets are formulated by, for example, preparing a powder mixture, granulating or dry-pressing the mixture, adding a lubricant and a disintegrant and pressing the entire mixture to give tablets. A powder mixture is prepared by mixing the compound comminuted in a suitable manner with a diluent or a base, as described above, and optionally with a binder, such as, for example, carboxymethylcellulose, an alginate, gelatine or polyvinylpyrrolidone, a dissolution retardant, such as, for example, paraffin, an absorption accelerator, such as, for example, a quaternary salt, and/or an absorbant, such as, for example, bentonite, kaolin or dicalcium phosphate. The powder mixture can be granulated by wetting it with a binder, such as, for example, syrup, starch paste, acadia mucilage or solutions of cellulose or polymer materials and pressing it through a sieve. As an alternative to granulation, the powder mixture can be run through a tabletting machine, giving lumps of non-uniform shape which are broken up to form granules. The granules can be lubricated by addition of stearic acid, a stearate salt, talc or mineral oil in order to prevent sticking to the tablet casting moulds. The lubricated mixture is then pressed to give tablets. The compounds according to the invention can also be combined with a free-flowing inert excipient and then pressed directly to give tablets without carrying out the granulation or dry-pressing steps. A transparent or opaque protective layer consisting of a shellac sealing layer, a layer of sugar or polymer material and a gloss layer of wax may be present. Dyes can be added to these coatings in order to be able to differentiate between different dosage units.

Oral liquids, such as, for example, solution, syrups and elixirs, can be prepared in the form of dosage units so that a given quantity comprises a pre-specified amount of the compound. Syrups can be prepared by dissolving the compound in an aqueous solution with a suitable flavour, while elixirs are prepared using a non-toxic alcoholic vehicle. Suspensions can be formulated by dispersion of the compound in a non-toxic vehicle. Solubilisers and emulsifiers, such as, for example, ethoxylated isostearyl alcohols and polyoxyethylene sorbitol ethers, preservatives, flavour additives, such as, for example, peppermint oil or natural sweeteners or saccharin, or other artificial sweeteners and the like, can likewise be added.

The dosage unit formulations for oral administration can, if desired, be encapsulated in microcapsules. The formulation can also be prepared in such a way that the release is extended or retarded, such as, for example, by coating or embedding of particulate material in polymers, wax and the like.

The compounds of the formula I and salts, solvates and physiologically functional derivatives thereof can also be administered in the form of liposome delivery systems, such as, for example, small unilamellar vesicles, large unilamellar vesicles and multilamellar vesicles. Liposomes can be formed from various phospholipids, such as, for example, cholesterol, stearylamine or phosphatidylcholines.

The compounds of the formula I and the salts, solvates and physiologically functional derivatives thereof can also be delivered using monoclonal anti-bodies as individual carriers to which the compound molecules are coupled. The compounds can also be coupled to soluble polymers as targeted medicament carriers. Such polymers may encompass polyvinylpyrrolidone, pyran copolymer, polyhydroxypropylmethacrylamidophenol, polyhydroxyethylaspartamidophenol or polyethylene oxide polylysine, substituted by palmitoyl radicals. The compounds may furthermore be coupled to a class of biodegradable polymers which are suitable for achieving controlled release of a medicament, for example polylactic acid, poly-epsilon-caprolactone, polyhydroxybutyric acid, polyorthoesters, polyacetals, polydihydroxypyrans, polycyanoacrylates and crosslinked or amphipathic block copolymers of hydrogels.

Pharmaceutical formulations adapted for transdermal administration can be administered as independent plasters for extended, close contact with the epidermis of the recipient. Thus, for example, the active ingredient can be delivered from the plaster by iontophoresis, as described in general terms in Pharmaceutical Research, 3(6), 318 (1986).

Pharmaceutical compounds adapted for topical administration can be formulated as ointments, creams, suspensions, lotions, powders, solutions, pastes, gels, sprays, aerosols or oils.

For the treatment of the eye or other external tissue, for example mouth and skin, the formulations are preferably applied as topical ointment or cream. In the case of formulation to give an ointment, the active ingredient can be employed either with a paraffinic or a water-miscible cream base. Alternatively, the active ingredient can be formulated to give a cream with an oil-in-water cream base or a water-in-oil base.

Pharmaceutical formulations adapted for topical application to the eye include eye drops, in which the active ingredient is dissolved or suspended in a suitable carrier, in particular an aqueous solvent.

Pharmaceutical formulations adapted for topical application in the mouth encompass lozenges, pastilles and mouthwashes.

Pharmaceutical formulations adapted for rectal administration can be administered in the form of suppositories or enemas.

Pharmaceutical formulations adapted for nasal administration in which the carrier substance is a solid comprise a coarse powder having a particle size, for example, in the range 20-500 microns, which is administered in the manner in which snuff is taken, i.e. by rapid inhalation via the nasal passages from a container containing the powder held close to the nose. Suitable formulations for administration as nasal spray or nose drops with a liquid as carrier substance encompass active-ingredient solutions in water or oil.

Pharmaceutical formulations adapted for administration by inhalation encompass finely particulate dusts or mists, which can be generated by various types of pressurised dispensers with aerosols, nebulisers or insufflators.

Pharmaceutical formulations adapted for vaginal administration can be administered as pessaries, tampons, creams, gels, pastes, foams or spray formulations.

Pharmaceutical formulations adapted for parenteral administration include aqueous and non-aqueous sterile injection solutions comprising antioxidants, buffers, bacteriostatics and solutes, by means of which the formulation is rendered isotonic with the blood of the recipient to be treated; and aqueous and non-aqueous sterile suspensions, which may comprise suspension media and thickeners. The formulations can be administered in single-dose or multidose containers, for example sealed ampoules and vials, and stored in freeze-dried (lyophilised) state, so that only the addition of the sterile carrier liquid, for example water for injection purposes, immediately before use is necessary.

Injection solutions and suspensions prepared in accordance with the recipe can be prepared from sterile powders, granules and tablets.

It goes without saying that, in addition to the above particularly mentioned constituents, the formulations may also comprise other agents usual in the art with respect to the particular type of formulation; thus, for example, formulations which are suitable for oral administration may comprise flavours.

A therapeutically effective amount of a compound of the formula I depends on a number of factors, including, for example, the age and weight of the human or animal, the precise condition which requires treatment, and its severity, the nature of the formulation and the method of administration, and is ultimately determined by the treating doctor or vet. However, an effective amount of a compound according to the invention for the treatment of neoplastic growth, for example colon or breast carcinoma, is generally in the range from 0.1 to 100 mg/kg of body weight of the recipient (mammal) per day and particularly typically in the range from 1 to 10 mg/kg of body weight per day. Thus, the actual amount per day for an adult mammal weighing 70 kg is usually between 70 and 700 mg, where this amount can be administered as a single dose per day or usually in a series of part-doses (such as, for example, two, three, four, five or six) per day, so that the total daily dose is the same. An effective amount of a salt or solvate or of a physiologically functional derivative thereof can be determined as the fraction of the effective amount of the compound according to the invention per se. It can be assumed that similar doses are suitable for the treatment of other conditions mentioned above.

The invention furthermore relates to medicaments comprising at least one compound of the formula I and/or pharmaceutically usable derivatives, solvates and stereoisomers thereof, including mixtures thereof in all ratios, and at least one further medicament active ingredient.

The invention also relates to a set (kit) consisting of separate packs of
(a) an effective amount of a compound of the formula I and/or pharmaceutically usable derivatives, solvates and stereoisomers thereof, including mixtures thereof in all ratios, and
(b) an effective amount of a further medicament active ingredient.

The set comprises suitable containers, such as boxes, individual bottles, bags or ampoules. The set may, for example, comprise separate ampoules, each containing an effective amount of a compound of the formula I and/or pharmaceutically usable derivatives, solvates and stereoisomers thereof, including mixtures thereof in all ratios,

and an effective amount of a further medicament active ingredient in dissolved or lyophilised form.

The medicaments from Table 1 are preferably, but not exclusively, combined with the compounds of the formula I. A combination of the formula I and medicaments from Table 1 can also be combined with compounds of the formula VI.

TABLE 1

| | | |
|---|---|---|
| Alkylating agents | Cyclophosphamide | Lomustine |
| | Busulfan | Procarbazine |
| | Ifosfamide | Altretamine |
| | Melphalan | Estramustine phosphate |
| | Hexamethylmelamine | Mechloroethamine |
| | Thiotepa | Streptozocin |
| | chloroambucil | Temozolomide |
| | Dacarbazine | Semustine |
| | Carmustine | |
| Platinum agents | Cisplatin | Carboplatin |
| | Oxaliplatin | ZD-0473 (AnorMED) |
| | Spiroplatin | Lobaplatin (Aetema) |
| | Carboxyphthalatoplatinum | Satraplatin (Johnson |
| | Tetraplatin | Matthey) |
| | Ormiplatin | BBR-3464 (Hoffrnann- |
| | Iproplatin | La Roche) |
| | | SM-11355 (Sumitomo) |
| | | AP-5280 (Access) |
| Antimetabolites | Azacytidine | Tomudex |
| | Gemcitabine | Trimetrexate |
| | Capecitabine | Deoxycoformycin |
| | 5-fluorouracil | Fludarabine |
| | Floxuridine | Pentostatin |
| | 2-chlorodesoxyadenosine | Raltitrexed |
| | 6-Mercaptopurine | Hydroxyurea |
| | 6-Thioguanine | Decitabine (SuperGen) |
| | Cytarabine | Clofarabine (Bioenvision) |
| | 2-fluorodesoxycytidine | Irofulven (MGI Pharrna) |
| | Methotrexate | DMDC (Hoffmann-La |
| | Idatrexate | Roche) |
| | | Ethynylcytidine (Taiho) |
| Topoisomerase | Amsacrine | Rubitecan (SuperGen) |
| inhibitors | Epirubicin | Exatecan mesylate |
| | Etoposide | (Daiichi) |
| | Teniposide or | Quinamed (ChemGenex) |
| | mitoxantrone | Gimatecan (Sigma- Tau) |
| | Irinotecan (CPT-11) | Diflomotecan (Beaufour- |
| | 7-Ethyl-10- | Ipsen) |
| | hydroxycamptothecin | TAS-103 (Taiho) |
| | Topotecan | Elsamitrucin (Spectrum) |
| | Dexrazoxanet | J-107088 (Merck & Co) |
| | (TopoTarget) | BNP-1350 (BioNumerik) |
| | Pixantrone (Novuspharrna) | CKD-602 (Chong Kun |
| | Rebeccamycin analogue | Dang) |
| | (Exelixis) | KW-2170(Kyowa Hakko) |
| | BBR-3576 (Novuspharrna) | |
| Antitumour | Dactinomycin (Actinomycin | Amonafide |
| antibiotics | D) | Azonafide |
| | Doxorubicin (Adriamycin) | Anthrapyrazole |
| | Deoxyrubicin | Oxantrazole |
| | Valrubicin | Losoxantrone |
| | Daunorubicin | Bleomycin sulfate |
| | (Daunomycin) | (Blenoxan) |
| | Epirubicin | Bleomycinic acid |
| | Therarubicin | Bleomycin A |
| | Idarubicin | Bleomycin B |
| | Rubidazon | Mitomycin C |
| | Plicamycinp | MEN-10755 (Menarini) |
| | Porfiromycin | GPX-100 (Gem |
| | Cyanomorpholinodoxo- | Pharmaceuticals) |
| | rubicin | |
| | Mitoxantron (Novantron) | |
| Antimitotic agents | Paclitaxel | SB 408075 |
| | Docetaxel | (GlaxoSmithKline) |
| | Colchicine | E7010 (Abbott) |
| | Vinblastine | PG-TXL (Cell |
| | Vincristine | Therapeutics) |
| | Vinorelbine | IDN 5109 (Bayer) |
| | Vindesine | A 105972 (Abbott) |
| | Dolastatin 10 (NCI) | A 204197 (Abbott) |
| | Rhizoxin (Fujisawa) | LU 223651 (BASF) |
| | Mivobulin (Warner- | D 24851 (ASTA Medica) |
| | Lambert) | ER-86526 (Eisai) |
| | Cemadotin (BASF) | Combretastatin A4 (BMS) |
| | RPR 109881A (Aventis) | Isohomohalichondrin-B |
| | TXD 258 (Aventis) | (PharmaMar) |
| | Epothilone B (Novartis) | ZD 6126 (AstraZeneca) |
| | T 900607 (Tularik) | PEG-Paclitaxel (Enzon) |
| | T 138067 (Tularik) | AZ10992 (Asahi) |
| | Cryptophycin 52 (Eli Lilly) | !DN-5109 (Indena) |
| | Vinflunine (Fabre) | AVLB (Prescient |
| | Auristatin PE (Teikoku | NeuroPharma) |
| | Hormone) | Azaepothilon B (BMS) |
| | BMS 247550 (BMS) | BNP-7787 (BioNumerik) |
| | BMS 184476 (BMS) | CA-4-Prodrug (OXiGENE) |
| | BMS 188797 (BMS) | Dolastatin-10 (NrH) |
| | Taxoprexin (Protarga) | CA-4 (OXiGENE) |
| Aromatase | Aminoglutethimide | Exemestan |
| inhibitors | Letrozole | Atamestan (BioMedicines) |
| | Anastrazole | YM-511 (Yamanouchi) |
| | Formestan | |
| Thymidylate | Pemetrexed (Eli Lilly) | Nolatrexed (Eximias) |
| synthase | ZD-9331 (BTG) | CoFactor ™ (BioKeys) |
| inhibitors | | |
| DNA antagonists | Trabectedin (PharmaMar) | Mafosfamide (Baxter |
| | Glufosfamide (Baxter | International) |
| | International) | Apaziquone (Spectrum |
| | Albumin + 32P (Isotope | Pharmaceuticals) |
| | Solutions) | O6-benzylguanine |
| | Thymectacin (NewBiotics) | (Paligent) |
| | Edotreotid (Novartis) | |
| Farnesyl | Arglabin (NuOncology | Tipifarnib (Johnson & |
| transferase | Labs) | Johnson) |
| inhibitors | Ionafarnib (Schering- | Perillyl alcohol (DOR |
| | Plough) | BioPharma) |
| | BAY-43-9006 (Bayer) | |
| Pump inhibitors | CBT-1 (CBA Pharma) | Zosuquidar |
| | Tariquidar (Xenova) | trihydrochloride (Eli Lilly) |
| | MS-209 (Schering AG) | Biricodar dicitrate (Vertex) |
| Histone acetyl | Tacedinaline (Pfizer) | Pivaloyloxymethyl butyrate |
| transferase | SAHA (Aton Pharma) | (Titan) |
| inhibitors | MS-275 (Schering AG) | Depsipeptide (Fujisawa) |
| Metalloproteinase | Neovastat (Aeterna | CMT -3 (CollaGenex) |
| inhibitors | Laboratories) | BMS-275291 (Celltech) |
| Ribonucleoside | Marimastat (British | Tezacitabine (Aventis) |
| reductase | Biotech) | Didox (Molecules for |
| inhibitors | Gallium maltolate (Titan) | Health) |
| | Triapin (Vion) | |
| TNF-alpha | Virulizin (Lorus | Revimid (Celgene) |
| agonists/ | Therapeutics) | |
| antagonists | CDC-394 (Celgene) | |
| Endothelin-A | Atrasentan (Abbot) | YM-598 (Yamanouchi) |
| receptor | ZD-4054 (AstraZeneca) | |
| antagonists | | |
| Retinoic acid | Fenretinide (Johnson & | Alitretinoin (Ligand) |
| receptor agonists | Johnson) | |
| | LGD-1550 (Ligand) | |
| Immuno- | Interferon | Dexosome therapy |
| modulators | Oncophage (Antigenics) | (Anosys) |
| | GMK (Progenics) | Pentrix (Australian Cancer |
| | Adenocarcinoma vaccine | Technology) |
| | (Biomira) | JSF-154 (Tragen) |
| | CTP-37 (AVI BioPharma) | Cancer vaccine (Intercell) |
| | JRX-2 (Immuno-Rx) | Norelin (Biostar) |
| | PEP-005 (Peplin Biotech) | BLP-25 (Biomira) |
| | Synchrovax vaccines (CTL | MGV (Progenics) |
| | Immuno) | !3-Alethin (Dovetail) |
| | Melanoma vaccine (CTL | CLL-Thera (Vasogen) |
| | Immuno) | |
| | p21-RAS vaccine | |
| | (GemVax) | |
| Hormonal and | Oestrogens | Prednisone |
| antihormonal | Conjugated oestrogens | Methylprednisolone |
| agents | Ethynyloestradiol | Prednisolone |
| | chlorotrianisene | Aminoglutethimide |
| | Idenestrol | Leuprolide |
| | Hydroxyprogesterone | Goserelin |
| | caproate | Leuporelin |
| | Medroxyprogesterone | Bicalutamide |
| | Testosterone | Flutamide |
| | Testosterone propionate | Octreotide |
| | Fluoxymesterone | Nilutamide |
| | Methyltestosterone | Mitotan |
| | Diethylstilbestrol | P-04 (Novogen) |
| | Megestrol | 2-Methoxyoestradiol |
| | Tamoxifen | (EntreMed) |
| | Toremofin | Arzoxifen (Eli Lilly) |
| | Dexamethasone | |
| Photodynamic | Talaporfin (Light Sciences) | Pd-Bacteriopheophorbid |
| agents | Theralux | (Yeda) |
| | (Theratechnologies) | Lutetium-Texaphyrin |
| | Motexafin-Gadolinium | (Pharmacyclics) |
| | (Pharmacyclics) | Hypericin |
| Tyrosine kinase | Imatinib (Novartis) | Kahalide F (PharmaMar) |
| inhibitors | Leflunomide(Sugen/Pharmacia) | CEP- 701 (Cephalon) |
| | | CEP-751 (Cephalon) |
| | ZDI839 (AstraZeneca) | MLN518 (Millenium) |
| | Erlotinib (Oncogene | PKC412 (Novartis) |
| | Science) | Phenoxodiol O |
| | Canertjnib (Pfizer) | Trastuzumab (Genentech) |
| | Squalamine (Genaera) | C225 (ImClone) |
| | SU5416 (Pharmacia) | rhu-Mab (Genentech) |
| | SU6668 (Pharmacia) | MDX-H210 (Medarex) |
| | ZD4190 (AstraZeneca) | 2C4 (Genentech) |
| | ZD6474 (AstraZeneca) | MDX-447 (Medarex) |
| | Vatalanib (Novartis) | ABX-EGF (Abgenix) |
| | PKI166(Novartis) | IMC-1C11 (ImClone) |
| | GW2016 | |
| | (GlaxoSmithKline) | |
| | EKB-509 (Wyeth) | |
| | EKB-569 (Wyeth) | |
| Various agents | SR-27897 (CCK-A | BCX-1777 (PNP inhibitor, |
| | inhibitor, Sanofi- | BioCryst) |
| | Synthelabo) | Ranpirnase (ribonuclease |
| | Tocladesine (cyclic AMP | stimulant, Alfacell) |
| | agonist, Ribapharm) | Galarubicin (RNA |
| | Alvocidib (CDK inhibitor, | synthesis inhibitor, Dong- |
| | Aventis) | A) |
| | CV-247 (COX-2 inhibitor, | Tirapazamine (reducing |
| | Ivy Medical) | agent, SRI International) |
| | P54 (COX-2 inhibitor, | N-Acetylcysteine (reducing |
| | Phytopharm) | agent, Zambon) |
| | CapCell ™ (CYP450 | R-Flurbiprofen (NF-kappaB |
| | stimulant, Bavarian Nordic) | inhibitor, Encore) |
| | GCS-IOO (gal3 antagonist, | 3CPA (NF-kappaB |
| | GlycoGenesys) | inhibitor, Active Biotech) |
| | G17DT immunogen | Seocalcitol (vitamin D |
| | (gastrin inhibitor, Aphton) | receptor agonist, Leo) |
| | Efaproxiral (oxygenator, | 131-I-TM-601 (DNA |
| | Allos Therapeutics) | antagonist, |
| | PI-88 (heparanase | TransMolecular) |
| | inhibitor, Progen) | Eflornithin (ODC inhibitor, |
| | Tesmilifen (histamine | ILEX Oncology) |
| | antagonist, YM | Minodronic acid |
| | BioSciences) | (osteoclast inhibitor, |
| | Histamine (histamine H2 | Yamanouchi) |
| | receptor agonist, Maxim) | Indisulam (p53 stimulant, |
| | Tiazofurin (IMPDH | Eisai) |
| | inhibitor, Ribapharm) | Aplidin (PPT inhibitor, |
| | Cilengitide (integrin | PharmaMar) |
| | antagonist, Merck KGaA) | Rituximab (CD20 antibody, |
| | SR-31747 (IL-1 antagonist, | Genentech) |
| | Sanofi-Synthelabo) | Gemtuzumab (CD33 |
| | CCI-779 (mTOR kinase | antibody, Wyeth Ayerst) |
| | inhibitor, Wyeth) | PG2 (haematopoiesis |
| | Exisulind (PDE-V inhibitor, | promoter, Pharmagenesis) |
| | Cell Pathways) | Immunol ™ (triclosan |
| | CP-461 (PDE-V inhibitor, | mouthwash, Endo) |
| | Cell Pathways) | Triacetyluridine (uridine |
| | AG-2037 (GART inhibitor, | prodrug, Wellstat) |
| | Pfizer) | SN-4071 (sarcoma agent, |
| | WX-UK1 (plasminogen | Signature BioScience) |
| | activator inhibitor, Wilex) | TransMID-107 ™ |
| | PBI-1402 (PMN stimulant, | (immunotoxin, KS |
| | ProMetic LifeSciences) | Biomedix) |
| | Bortezomib (proteasome | PCK-3145 (apoptosis |
| | inhibitor, Millennium) | promoter, Procyon) |
| | SRL-172 (T-cell stimulant, | Doranidazole (apoptosis |
| | SR Pharma) | promoter, Pola) |
| | TLK-286 (glutathione-S | CHS-828 (cytotoxic agent, |
| | transferase inhibitor, Telik) | Leo) |
| | PT-100 (growth factor | Trans-retinic acid |
| | agonist, Point | (differentiator, NIH) |
| | Therapeutics) | MX6 (apoptosis promoter, |
| | Midostaurin (PKC inhibitor, | MAXIA) |
| | Novartis) | Apomine (apoptosis |
| | Bryostatin-1 (PKC | promoter, ILEX Oncology) |
| | stimulant, GPC Biotech) | Urocidin (apoptosis |
| | CDA-II (apoptosis | promoter, Bioniche) |
| | promoter, Everlife) | Ro-31-7453 (apoptosis |
| | SDX-101 (apoptosis | promoter, La Roche) |
| | promoter, Salmedix) | Brostallicin (apoptosis |
| | Ceflatonin (apoptosis | promoter, Pharmacia) |
| | promoter, ChemGenex) | |

The compounds of the formula I are preferably combined with known anti-cancer agents:

The present compounds are also suitable for combination with known anti-cancer agents. These known anti-cancer agents include the following: oestrogen receptor modulators, androgen receptor modulators, retinoid receptor modulators, cytotoxic agents, antiproliferative agents, prenyl-protein transferase inhibitors, HMG-CoA reductase inhibitors, HIV protease inhibitors, reverse transcriptase inhibitors and other angiogenesis inhibitors. The present compounds are particularly suitable for administration at the same time as radiotherapy. The synergistic effects of inhibition of VEGF in combination with radiotherapy have been described by specialists (see WO 00/61186). “Oestrogen receptor modulators” refers to compounds which interfere with or inhibit the binding of oestrogen to the receptor, regardless of mechanism. Examples of oestrogen receptor modulators include, but are not limited to, tamoxifen, raloxifene, idoxifene, LY353381, LY 117081, toremifene, fulvestrant, 4-[7-(2,2-dimethyl-1-oxopropoxy-4-methyl-2-[4-[2-(1-piperidinyl)ethoxy]phenyl]-2H-1-benzopyran-3-yl]phenyl 2,2-dimethylpropanoate, 4,4′-dihydroxybenzophenone-2,4-dinitrophenylhydrazone and SH646.

“Androgen receptor modulators” refers to compounds which interfere with or inhibit the binding of androgens to the receptor, regardless of mechanism. Examples of androgen receptor modulators include finasteride and other 5α-reductase inhibitors, nilutamide, flutamide, bicalutamide, liarozole and abiraterone acetate.

“Retinoid receptor modulators” refers to compounds which interfere with or inhibit the binding of retinoids to the receptor, regardless of mechanism. Examples of such retinoid receptor modulators include bexarotene, tretinoin, 13-cis-retinoic acid, 9-cis-retinoic acid, α-difluoromethylornithine, ILX23-7553, trans-N-(4′-hydroxyphenyl)retinamide and N-4-carboxyphenylretinamide.

“Cytotoxic agents” refers to compounds which result in cell death primarily through direct action on the cellular function or inhibit or interfere with cell myosis, including alkylating agents, tumour necrosis factors, intercalators, microtubulin inhibitors and topoisomerase inhibitors.

Examples of cytotoxic agents include, but are not limited to, tirapazimine, sertenef, cachectin, ifosfamide, tasonermin, lonidamine, carboplatin, altretamine, prednimustine, dibromodulcitol, ranimustine, fotemustine, nedaplatin, oxaliplatin, temozolomide, heptaplatin, estramustine, improsulfan tosylate, trofosfamide, nimustine, dibrospidium chloride, pumitepa, lobaplatin, satraplatin, profiromycin, cisplatin, irofulven, dexifosfamide, cis-aminedichloro(2-methylpyridine)platinum, benzylguanine, glufosfamide, GPX100, (trans,trans,trans)bis-mu-(hexane-1,6-diamine)mu-[diamineplatinum(II)]bis-[diamine(chloro)platinum(II)]tetrachloride, diarisidinylspermine, arsenic trioxide, 1-(11-dodecylamino-10-hydroxyundecyl)-3,7-dimethylxanthine, zorubicin, idarubicin, daunorubicin, bisantrene, mitoxantrone, pirarubicin, pinafide, valrubicin, amrubicin, antineoplastone, 3′-deamino-3′-morpholino-13-deoxo-10-hydroxycaminomycin, annamycin, galarubicin, elinafide, MEN10755 and 4-demethoxy-3-deamino-3-aziridinyl-4-methylsulfonyldaunorubicin (see WO 00/50032).

Examples of microtubulin inhibitors include paclitaxel, vindesine sulfate, 3′,4′-didehydro-4′-deoxy-8′-norvincaleukoblastine, docetaxol, rhizoxin, dolastatin, mivobulin isethionate, auristatin, cemadotin, RPR109881, BMS184476, vinflunine, cryptophycin, 2,3,4,5,6-pentafluoro-N-(3-fluoro-4-methoxyphenyl)benzenesulfonamide, anhydrovinblastine, N,N-dimethyl-L-valyl-Lvalyl-N-methyl-L-valyl-L-prolyl-L-proline-t-butylamide, TDX258 and BMS188797.

Some examples of topoisomerase inhibitors are topotecan, hycaptamine, irinotecan, rubitecan, 6-ethoxypropionyl-3′,4′-O-exobenzylidenechartreusin, 9-methoxy-N,N-dimethyl-5-nitropyrazolo[3,4,5-kl]acridine-2-(6H)propanamine, 1-amino-9-ethyl-5-fluoro-2,3-dihydro-9-hydroxy-4-methyl-1H,12H-benzo[de]pyrano[3′,4′:b,7]indolizino[1,2b]quinoline-10,13(9H,15H)dione, lurtotecan, 7-[2-(N-isopropylamino)ethyl]-(20S)camptothecin, BNP1350, BNPI1100, BN80915, BN80942, etoposide phosphate, teniposide, sobuzoxane, 2′-dimethylamino-2′-deoxyetoposide, GL331, N-[2-(dimethylamino)ethyl]-9-hydroxy-5,6-dimethyl-6H-pyrido[4,3-b]carbazole-1-carboxamide, asulacrine, (5a,5aB,8aa,9b)-9-[2-[N-[2-(dimethylamino)ethyl]-N-methylamino]ethyl]-5-[4-hydroxy-3,5-dimethoxyphenyl]-5,5a,6,8,8a,9-hexohydrofuro(3′,4′:6,7)naphtho(2,3-d)-1,3-dioxol-6-one, 2,3-(methylenedioxy)-5-methyl-7-hydroxy-8-methoxybenzo[c]phenanthridinium, 6,9-bis[(2-aminoethyl)amino]benzo[g]isoquinoline-5,10-dione, 5-(3-aminopropylamino)-7,10-dihydroxy-2-(2-hydroxyethylaminomethyl)-6H-pyrazolo[4,5,1-de]acridin-6-one, N-[1-[2(diethylamino)ethylamino]-7-methoxy-9-oxo-9H-thioxanthen-4-ylmethyl]formamide, N-(2-(dimethylamino)ethyl)acridine-4-carboxamide, 6-[[2-(dimethylamino)ethyl]amino]-3-hydroxy-7H-indeno[2,1-c]quinolin-7-one and dimesna.

“Antiproliferative agents” include antisense RNA and DNA oligonucleotides such as G3139, ODN698, RVASKRAS, GEM231 and INX3001 and anti-metabolites such as enocitabine, carmofur, tegafur, pentostatin, doxifluridine, trimetrexate, fludarabine, capecitabine, galocitabine, cytarabine ocfosfate, fosteabine sodium hydrate, raltitrexed, paltitrexid, emitefur, tiazofurin, decitabine, nolatrexed, pemetrexed, nelzarabine, 2′-deoxy-2′-methylidenecytidine, 2′-fluoromethylene-2′-deoxycytidine, N-[5-(2,3-dihydrobenzofuryl)sulfonyl]-N′-(3,4-dichlorophenyl)urea, N6-[4-deoxy-4-[N2-[2(E),4(E)-tetradecadienoyl]glycylamino]-L-glycero-B-L-mannoheptopyranosyl]adenine, aplidine, ecteinascidin, troxacitabine, 4-[2-amino-4-oxo-4,6,7,8-tetrahydro-3H-pyrimidino[5,4-b]-1,4-thiazin-6-yl-(S)-ethyl]-2,5-thienoyl-L-glutamic acid, aminopterin, 5-fluorouracil, alanosine, 11-acetyl-8-(carbamoyloxymethyl)-4-formyl-6-methoxy-14-oxa-1,11-diazatetracyclo(7.4.1.0.0)tetradeca-2,4,6-trien-9-ylacetic acid ester, swainsonine, lometrexol, dexrazoxane, methioninase, 2′-cyano-2′-deoxy-N4-palmitoyl-1-B-D-arabinofuranosyl cytosine and 3-aminopyridine-2-carboxaldehyde thiosemicarbazone. “Antiproliferative agents” also include monoclonal antibodies to growth factors other than those listed under “angiogenesis inhibitors”, such as trastuzumab, and tumour suppressor genes, such as p53, which can be delivered via recombinant virus-mediated gene transfer (see U.S. Pat. No. 6,069,134, for example).

Particular preference is given to the use of the compound according to the invention for the treatment and prophylaxis of tumour diseases.

The tumour is preferably selected from the group of tumours of the squamous epithelium, bladder, stomach, kidneys, head and neck, oesophagus, cervix, thyroid, intestine, liver, brain, prostate, urogenital tract, lymphatic system, stomach, larynx and/or lung.

The tumour is furthermore preferably selected from the group lung adenocarcinoma, small-cell lung carcinomas, pancreatic cancer, glioblastomas, colon carcinoma and breast carcinoma.

Preference is furthermore given to the use for the treatment of a tumour of the blood and immune system, preferably for the treatment of a tumour selected from the group of acute myeloid leukaemia, chronic myeloid leukaemia, acute lymphatic leukaemia and/or chronic lymphatic leukaemia.

The invention also encompasses a method for the treatment of a patient who has a neoplasm, such as a cancer, by administration of

a) one or more of the compounds of the formula I:
b) and one or more of the compounds of the formula VI or acid-addition salts thereof, in particular hydrochlorides:

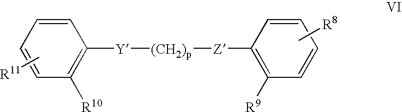

in which Y′ and Z′ each, independently of one another, denote O or N, R⁹ and R¹⁰ each, independently of one another, denote H, OH, halogen, OC1-10-alkyl, OCF₃, NO₂ or NH₂, n denotes an integer between 2 and 6, each inclusive, and R⁸ and R¹¹ are each, independently of one another, preferably in the meta- or para-position and are selected from the group:

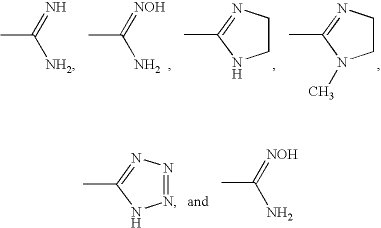

where the first and second compound are administered simultaneously or within 14 days of one another in amounts which are sufficient to inhibit the growth of the neoplasm.

The combination of the compounds of the formula I with the compounds of the formula VI and other pentamedine analogues results in a synergistic action in the inhibition of neoplasias. Combinations comprising the compounds of the formula VI are mentioned, for example, in WO 02058684.

The mechanism of action of pentamidine or derivatives thereof has not been clearly explained at present: pentamidine or derivatives thereof appears to have pleiotropic actions since it results in a decrease in DNA, RNA and protein synthesis. It was recently described that pentamidine is a capable inhibitor of PRL1, -2 and 3 phosphatases (Pathak et al., 2002) and tyrosine phosphatases, and overexpression thereof is accompanied by neoplastic malignant tumours in humans. On the other hand, it has been described that pentamidine is a medicament which binds to the DNA minor groove (Puckowska et al., 2004) and is able to exert its action via disturbance of gene expression and/or DNA synthesis.

Experiments show that:
both pentamidine and also preferably the compounds of the formula I maintain cells in the G2/M cell cycle.
the combination of pentamidine and compounds of the formula I preferably have additive to synergistic actions on cell proliferation.

Other suitable pentamidine analogues include stilbamidine (G-1) and hydroxystilbamidine (G-2) and indole analogues thereof (for example G-3):

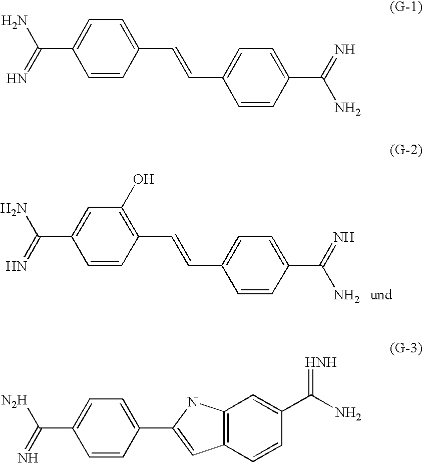

Each amidine unit may be replaced, independently of one another, by one of the units defined above for R⁸ and R¹¹. As in the case of benzimidazoles and pentamidines, salts of stilbamidine, hydroxystilbamidine and indole derivatives thereof are also suitable for the process according to the invention. Preferred salts include, for example, dihydrochloride and methanesulfonate salts.

Still other analogues are those which fall under a formula which are provided in one of the U.S. Pat. Nos. 5,428,051, 5,521,189, 5,602,172, 5,643,935, 5,723,495, 5,843,980, 6,172,104 and 6,326,395 or the US patent application with the publication No. US 2002/0019437 A1, each of which is incorporated in its entirety by way of reference. Illustrative analogues include 1,5-bis(4′-(N-hydroxyamidino)phenoxy)pentane, 1,3-bis(4′-(N-hydroxyamidino)phenoxy)propane, 1,3-bis(2′-methoxy-4′-(N-hydroxyamidino)phenoxy)propane, 1,4-bis(4′-(N-hydroxyamidino)phenoxy)butane, 1,5-bis(4′-(N-hydroxyamidino)phenoxy)pentane, 1,4-bis(4′-(N-hydroxyamidino)phenoxy)butane, 1,3-bis(4′-(4-hydroxyamidino)phenoxy)propane, 1,3-bis(2′-methoxy-4′-(N-hydroxyamidino)phenoxy)propane, 2,5-bis[4-amidinophenyl]furan, 2,5-bis[4-amidinophenyl]furan bisamide oxime, 2,5-bis[4-amidinophenyl]furan bis-O-methylamide oxime, 2,5-bis[4-amidinophenyl]furan bis-O-ethylamide oxime, 2,8-diamidinodibenzothiophene, 2,8-bis(N-isopropylamidino)carbazole, 2,8-bis(N-hydroxyamidino)carbazole, 2,8-bis(2-imidazolinyl)dibenzothiophene, 2,8-bis(2-imidazolinyl)-5,5-dioxodibenzothiophene, 3,7-diamidinodibenzothiophene, 3,7-bis(N-isopropylamidino)dibenzothiophene, 3,7-bis(N-hydroxyamidino)dibenzothiophene, 3,7-diaminodibenzothiophene, 3,7-dibromodibenzothiophene, 3,7-dicyanodibenzothiophene, 2,8-diamidinodibenzofuran, 2,8-di-(2-imidazolinyl)dibenzofuran, 2,8-di-(N-isopropylamidino)dibenzofuran, 2,8-di(N-hydroxylamidino)dibenzofuran, 3,7-di-(2-imidazolinyl)dibenzofuran, 3,7-di(isopropylamidino)dibenzofuran, 3,7-di-(A-hydroxylamidino)dibenzofuran, 2,8-dicyanodibenzofuran, 4,4′-dibromo-2,2′-dinitrobiphenyl, 2-methoxy-2′-nitro-4,4′-dibromobiphenyl, 2-methoxy-2′-amino-4,4′-dibromobiphenyl, 3,7-dibromodibenzofuran, 3,7-dicyanodibenzofuran, 2,5-bis(5-amidino-2-benzimidazolyl)pyrrole, 2,5-bis[5-(2-imidazolinyl)-2-benzimidazolyl]pyrrole, 2,6-bis[5-(2-imidazolinyl)-2-benzimidazolyl]pyridine, 1-methyl-2,5-bis(5-amidino-2-benzimidazolyl)pyrrole, 1-methyl-2,5-bis[5-(2-imidazolyl)-2-benzimidazolyl]pyrrole, 1-methyl-2,5-bis[5-(1,4,5,6-tetrahydro-2-pyrimidinyl)-2-benzimidazolyl]pyrrole, 2,6-bis(5-amidino-2-benzimidazoyl)pyridine, 2,6-bis[5-(1,4,5,6-tetrahydro-2-pyrimidinyl)-2-benzimidazolyl]pyridine, 2,5-bis(5-amidino-2-benzimidazolyl)furan, 2,5-bis[5-(2-imidazolinyl)-2-benzimidazolyl]-furan, 2,5-bis(5-N-isopropylamidino-2-benzimidazolyl)furan, 2,5-bis(4-guanylphenyl)furan, 2,5-bis(4-guanylphenyl)-3,4-dimethylfuran, 2,5-di-p-[2-(3,4,5,6-tetrahydropyrimidyl)phenyl]furan, 2,5-bis[4-(2-imidazolinyl)phenyl]furan, 2,5-[bis{4-(2-tetrahydropyrimidinyl)}phenyl]ₚ-(tolyloxy)furan, 2,5-[bis{4-(2-imidazolinyl)}phenyl]-3-p-(tolyloxy)furan, 2,5-bis{4-[5-(N-2-aminoethylamido)benzimidazol-2-yl]phenyl}furan, 2,5-bis[4-(3a,4,5,6,7,7a-hexahydro-1H-benzimidazol-2-yl)phenyl]furan, 2,5-bis[4-(4,5,6,7-tetrahydro-1H-1,3-diazepin-2-yl)phenyl]furan, 2,5-bis(4-N,N-dimethylcarboxhydrazidophenyl)furan, 2,5-bis{4-[2-(N-2-hydroxyethyl)imidazolinyl]phenyl}furan, 2,5-bis[4-(N-isopropylamidino)phenyl]furan, 2,5-bis{4-[3-(dimethylaminopropyl)amidino]phenyl}-furan, 2,5-bis{4-[N-(3-aminopropyl)amidino]phenyl}furan, 2,5-bis[2-(imidzaolinyl)phenyl]-3,4-bis(methoxymethyl)furan, 2,5-bis[4-N-(dimethylaminoethyl)guanyl]phenylfuran, 2,5-bis{4-[(N-2-hydroxyethyl)guanyl]phenyl}furan, 2,5-bis[4-N-(cyclopropylguanyl)phenyl]furan, 2,5-bis[4-(N,N-diethylaminopropyl)guanyl]phenylfuran, 2,5-bis{4-[2-(N-ethylimidazolinyl)]phenyl}furan, 2,5-bis{4-[N-(3-pentylguanyl)]}phenylfuran, 2,5-bis[4-(2-imidazolinyl)phenyl]-3-methoxyfuran, 2,5-bis[4-(N-isopropylamidino)phenyl]-3-methylfuran, bis[5-amidino-2-benzimidazolyl]methane, bis[5-(2-imidazolyl)-2-benzimidazolyl]-methane, 1,2-bis[5-amidino-2-benzimidazolyl]ethane, 1,2-bis[5-(2-imidazolyl)-2-benzimidazolyl]ethane, 1,3-bis[5-amidino-2-benzimidazolyl]propane, 1,3-bis[5-(2-imidazolyl)-2-benzimidazolyl]propane, 1,4-bis[5-amidino-2-benzimidazolyl]propane, 1,4-bis[5-(2-imidazolyl)-2-benzimidazolyl]butane, 1,8-bis[5-amidino-2-benzimidazolyl]octane, trans-1,2-bis[5-amidino-2-benzimidazolyl]ethene, 1,4-bis[5-(2-imidazolyl)-2-benzimidazolyl]-1-butene, 1,4-bis[5-(2-imidazolyl)-2-benzimidazolyl]-2-butene, 1,4-bis[5-(2-imidazolyl)-2-benzimidazolyl]-1-methylbutane, 1,4-bis[5-(2-imidazolyl)-2-benzimidazolyl]-2-ethylbutane, 1,4-bis[5-(2-imidazolyl)-2-benzimidazolyl]-1-methyl-1-butene, 1,4-bis[5-(2-imidazolyl)-2-benzimidazolyl]-2,3-diethyl-2-butene, 1,4-bis[5-(2-imidazolyl)-2-benzimidazolyl]-1,3-butadiene, 1,4-bis[5-(2-imidazolyl)-2-benzimidazolyl]-2-methyl-1,3-butadiene, bis[5-(2-pyrimidyl)-2-benzimidazolyl]-methane, 1,2-bis[5-(2-pyrimidyl)-2-benzimidazolyl]ethane, 1,3-bis[5-amidino-2-benzimidazolyl]propane, 1,3-bis[5-(2-pyrimidyl)-2-benzimidazolyl]propane, 1,4-bis[5-(2-pyrimidyl)-2-benzimidazolyl]butane, 1,4-bis[5-(2-pyrimidyl)-2-benzimidazolyl]-1-butene, 1,4-bis[5-(2-pyrimidyl)-2-benzimidazolyl]-2-butene, 1,4-bis[5-(2-pyrimidyl)-2-benzimidazolyl]-1-methylbutane, 1,4-bis[5-(2-pyrimidyl)-2-benzimidazolyl]-2-ethylbutane, 1,4-bis[5-(2-pyrimidyl)-2-benzimidazolyl]-1-methyl-1-butene, 1,4-bis[5-(2-pyrimidyl)-2-benzimidazolyl]-2,3-diethyl-2-butene, 1,4-bis[5-(2-pyrimidyl)-2-benzimidazolyl]-1,3-butadiene and 1,4-bis[5-(2-pyrimidyl)-2-benzimidazolyl]-2-methyl-1,3-butadiene, 2,4-bis(4-guanylphenyl)pyrimidine, 2,4-bis(4-imidazolin-2-yl)pyrimidine, 2,4-bis[(tetrahydropyrimidinyl-2-yl)phenyl]pyrimidine, 2-(4-[N-i-propylguanyl]phenyl)-4-(2-methoxy-4-[N-i-propylguanyl]phenyl)pyrimidine, 4-(N-cyclopentylamidino)-1,2-phenylenediamine, 2,5-bis[2-(5-amidino)benzimidazoyl]furan, 2,5-bis[2-{5-(2-imidazolino)}benzimidazoyl]furan, 2,5-bis[2-(5-N-isopropylamidino)benzimidazoyl]furan, 2,5-bis[2-(5-N-cyclopentylamidino)benzimidazoyl]furan, 2,5-bis[2-(5-amidino)benzimidazoyl]pyrrole, 2,5-bis[2-{5-(2-imidazolino)}benzimidazoyl]pyrrole, 2,5-bis[2-(5-N-isopropylamidino)benzimidazoyl]pyrrole, 2,5-bis[2-(5-N-cyclopentylamidino)benzimidazoyl]pyrrole, 1-methyl-2,5-bis[2-(5-amidino)benzimidazoyl]pyrrole, 2,5-bis[2-{5-(2-imidazolino)}benzimidazoyl]-1-methylpyrrole, 2,5-bis[2-(5-N-cyclopentylamidino)benzimidazoyl]-1-methylpyrrole, 2,5-bis[2-(5-N-isopropylamidino)benzimidazoyl]thiophene, 2,6-bis[2-{5-(2-imidazolino)}benzimidazoyl]pyridine, 2,6-bis[2-(5-amidino)benzimidazoyl]pyridine, 4,4′-bis[2-(5-N-isopropylamidino)benzimidazoyl]-1,2-diphenylethane, 4,4′-bis[2-(5-N-cyclopentylamidino)benzimidazoyl]-2,5-diphenylfuran, 2,5-bis[2-(5-amidino)benzimidazoyl]benzo[b]furan, 2,5-bis[2-(5-N-cyclopentylamidino)benzimidazoyl]benzo[b]furan, 2,7-bis[2-(5-N-isopropylamidino)benzimidazoyl]fluorine, 2,5-bis[4-(3-(N-morpholinopropyl)carbamoyl)phenyl]furan, 2,5-bis[4-(2-N,N-dimethylaminoethylcarbamoyl)phenyl]furan, 2,5-bis[4-(3-N,N-dimethylaminopropylcarbamoyl)phenyl]furan, 2,5-bis[4-(3-N-methyl-3-N-phenylaminopropylcarbamoyl)phenyl]furan, 2,5-bis[4-(3-N,N8,N11-trimethylaminopropylcarbamoyl)phenyl]furan, 2,5-bis[3-amidinophenyl]furan, 2,5-bis[3-(N-isopropylamidino)amidinophenyl]furan, 2,5-bis[3-[(N-(2-dimethylaminoethyl)amidino]phenylfuran, 2,5-bis[4-(N-2,2,2-trichloroethoxycarbonyl)amidinophenyl]furan, 2,5-bis[4-(N-thioethylcarbonyl)amidinophenyl]furan, 2,5-bis[4-(N-benzyloxycarbonyl)amidinophenyl]furan, 2,5-bis[4-(N-phenoxycarbonyl)amidinophenyl]furan, 2,5-bis[4-(N-(4-fluoro)phenoxycarbonyl)amidinophenyl]furan, 2,5-bis[4-(N-(4-methoxy)phenoxycarbonyl)amidinophenyl]furan, 2,5-bis[4-(1-acetoxyethoxycarbonyl)amidinophenyl]furan and 2,5-bis[4-(N-(3-fluoro)phenoxycarbonyl)amidinophenyl]-furan. Processes for the preparation of one of the above compounds are described in U.S. Pat. Nos. 5,428,051, 5,521,189, 5,602,172, 5,643,935, 5,723,495, 5,843,980, 6,172,104 and 6,326,395 or the US patent application with the publication no. US 2002/0019437 A1.

Pentamidine metabolites are likewise suitable in the antiproliferative combination according to the invention. Pentamidine is rapidly metabolised in the body to at least seven primary metabolites. Some of these metabolites have one or more actions in common with pentamidine Pentamidine metabolites have an antiproliferative action when combined with a benzimidazole or an analogue thereof.

Seven pentamidine analogues are shown below.

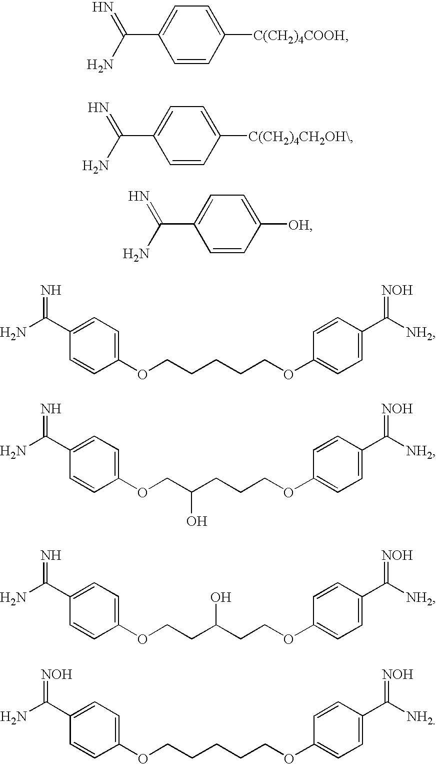

The combinations according to the invention of compounds of the formula I and formula VI or analogues thereof and metabolites thereof are suitable for the treatment of neoplasms. Combination therapy can be carried out alone or in combination with another therapy (for example operation, irradiation, chemotherapy, biological therapy). In addition, a person whose risk of developing a neoplasm is greater (for example someone who is genetically pre-disposed or someone who previously had a neoplasm) can be given prophylactic treatment in order to inhibit or delay neoplasm formation.

The invention likewise relates to the combination of kinesin ATPase Eg5/KSP with the compounds of the formula V, pentamidine, analogues thereof and/or metabolites thereof.

The dosage and frequency of administration of each compound in the combination can be controlled independently. For example, one compound may be administered orally three times daily, while the second compound may be administered intramuscularly once per day. The compounds may also be formulated together, leading to administration of both compounds.

The antiproliferative combinations according to the invention can also be provided as components of a pharmaceutical package. The two medicaments can be formulated together or separately and in individual dosage amounts.

Under another aspect, the invention encompasses a method for the treatment of a patient who has a neoplasm, such as a cancer, by administration of a compound of the formula (I) and (V) in combination with an antiproliferative agent. Suitable antiproliferative agents encompass those provided in Table 1. The following examples are intended to help the person skilled in the art to understand the present invention better with reference to the experiments explained specifically below and the results of these experiments. However, the examples are not intended to restrict the present invention and the scope of protection defined by the patent claims. The characteristics, features, properties, advantages and/or applications given in the examples for the compounds mentioned in the examples can also be applied to other compounds, which are not mentioned in the examples, applications and other subject-matters according to the invention covered by the patent claims.

Above and below, all temperatures are indicated in ° C. In the following examples, “conventional work-up” means: if necessary, water is added, the pH is adjusted, if necessary, to values between 2 and 10, depending on the constitution of the end product, the mixture is extracted with ethyl acetate or dichloromethane, the phases are separated, the organic phase is dried over sodium sulfate and evaporated, and the product is purified by chromatography on silica gel and/or by crystallisation. R^{f} values on silica gel; eluent: ethyl acetate/methanol 9:1.

Mass spectrometry (MS): EI (electron impact ionisation) M⁺

FAB (fast atom bombardment) (M+H)⁺

ESI (electrospray ionisation) (M+H)⁺

APCI-MS (atmospheric pressure chemical ionisation—mass spectrometry) (M+H)⁺

### EXAMPLES

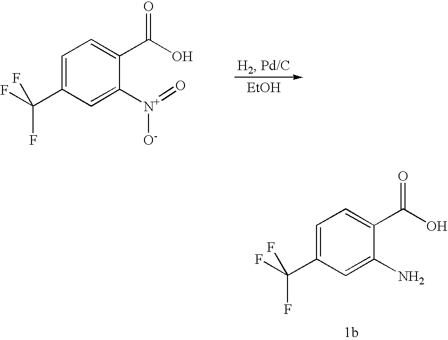

10 g (42.53 mmol) of 2-nitro-4-trifluoromethylbenzoic acid are hydrogenated using Pd/C (5%) in ethanol at room temperature until the reaction is complete. The reaction solution is filtered through kieselguhr, rinsed with EtOH, and the filtrate is subsequently evaporated. The residue is recrystallised from ethanol.

Yield: 8.5 g (97%) of 1b, pale-yellow crystals

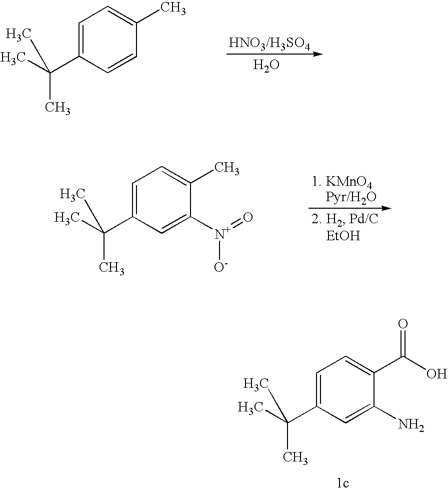

For the preparation of the nitrating acid, 4.4 ml of fuming nitric acid are dissolved in 3.6 ml of ice-water with ice cooling. 12 ml of concentrated sulfuric acid are subsequently added in portions at such a rate that the temperature can be kept between 5 and 10° C.

12 ml (70 mmol) of 4-tert-butyltoluene are initially introduced, cooled using an ice bath, and the still-cold nitrating acid is added dropwise in portions with vigorous stirring. The temperature is kept between 5 and 10° C. during this operation. When the addition is complete, the reaction mixture is stirred at room temperature for a further 1 h, subsequently poured onto ice and extracted 4 times with diethyl ether. The combined organic phases are washed with water until neutral, dried using Na₂SO₄, filtered and evaporated. The residue is purified by chromatography (400 g of silica gel, eluent: petroleum ether/DCM 9:1).

Yield: 9.53 g (71%) of 2-nitro-4-tert-butyltoluene, pale-yellow oil

4 g (21 mmol) of 2-nitro-4-tert-butyltoluene are suspended in 50 ml of pyridine/water—1/1 and heated to the boiling point (98° C.). 33 g (209 mmol) of potassium permanganate are then added in a number of small portions, and the reaction mixture is stirred under reflux for a further 3 h. The reaction mixture is cooled to room temperature, 10 ml of ethanol are added, and 20 ml of 1N NaOH are added. The solid is filtered off with suction and rinsed with water. The filtrate is extracted twice with ethyl acetate. The aqueous phase is subsequently acidified (pH 5) and extracted with ethyl acetate. The combined organic phases are washed with water until neutral, dried using Na₂SO₄, filtered and evaporated. The yellow oily residue crystallises on standing.

Yield: 4.4 g (94%) of 2-nitro-4-tert-butylbenzoic acid, yellow solid

8.6 g (38.14 mmol) of 2-nitro-4-tert-butylbenzoic acid are hydrogenated using Pd/C (5%) in ethanol at room temperature until the reaction is complete. The reaction solution is filtered through kieselguhr, rinsed with EtOH, and the filtrate is subsequently evaporated.

Yield: 6.35 g (80%) of 1c, brown solid

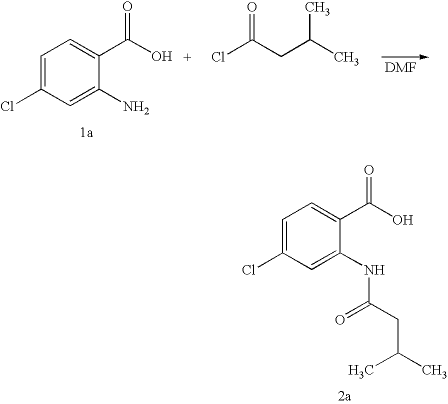

A solution of 39 ml (0.32 mol) of isovaleryl chloride in 40 ml of DMF is added dropwise to a solution of 50 g (0.29 mol) of 2-amino-4-chlorobenzoic acid 1a in 160 ml of dimethylformamide at such a rate that the temperature does not rise above 40° C. The reaction mixture is stirred at room temperature for a further 2 h and subsequently stirred into about 1.5 l of water. The precipitated product is filtered off with suction, washed with water and dried in vacuo.

Yield: 49.7 g (67%) of 2a, pale-yellow crystals

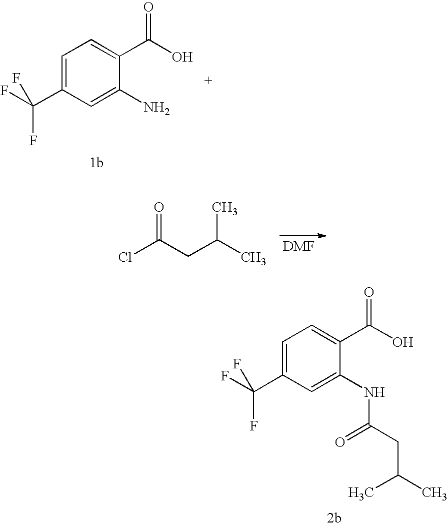

A solution of 3.3 ml (26.81 mol) of isovaleryl chloride in 5 ml of DMF is added dropwise to a solution of 5 g (24.37 mmol) of 2-amino-4-trifluoromethylbenzoic acid 1b in 30 ml of dimethylformamide at such a rate that the temperature does not rise above 40° C. The reaction mixture is stirred at room temperature for a further 2 h and subsequently stirred into about 120 ml of water. The precipitated product is filtered off with suction, taken up in ethyl acetate and washed with NaHCO₃ solution, water and saturated NaCl solution, dried using sodium sulfate, filtered and evaporated.

Yield: 4.11 g (58%) of 2b, yellow-brown oil

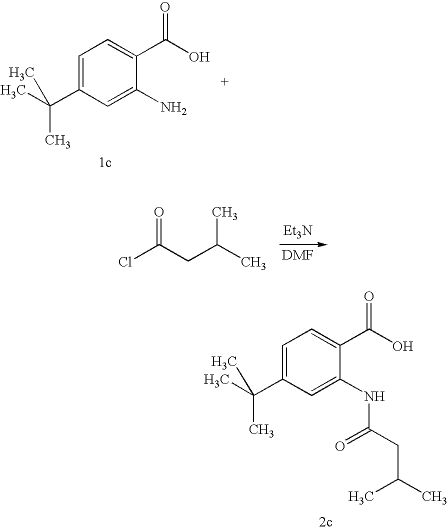

A solution of 2.6 ml (21.22 mol) of isovaleryl chloride in 3 ml of DMF is added dropwise at 0-5° C. to a solution of 3.73 g (19.29 mmol) of 2-amino-4-tert-butylbenzoic acid 1c and 2.94 ml (21.22 mmol) of triethylamine in 23 ml of dimethylformamide, and the reaction mixture is subsequently warmed to room temperature. After addition of a further 0.78 ml (6.37 mmol) of isovaleryl chloride, the reaction mixture is stirred at room temperature for a further 1 h, subsequently stirred into about 120 ml of water and extracted with ethyl acetate. The combined organic phases are washed with NaHCO₃ solution, water and saturated NaCl solution, dried using sodium sulfate, filtered and evaporated.

Yield: 1.9 g (36%) of 2c, brown oil

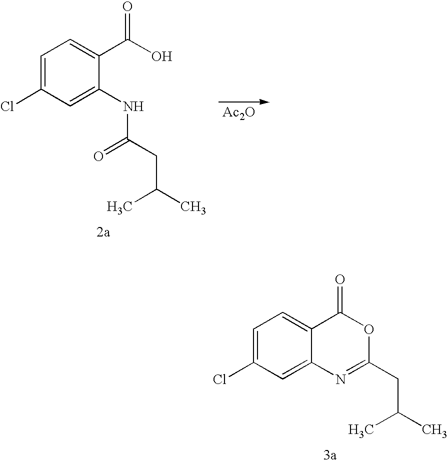

49 g (0.19 mol) of 2a are suspended in 170 ml of acetic anhydride and heated, during which a clear solution formed. The acetic acid formed is distilled off from the reaction mixture during the reaction. After 2 h, the reaction mixture is cooled to room temperature and evaporated to dryness in a rotary evaporator. The oily residue crystallises on standing in the refrigerator. The crystals are triturated with petroleum ether, filtered off with suction and dried.

Yield: 39.1 g (86%) of 3a, pale-brown crystals

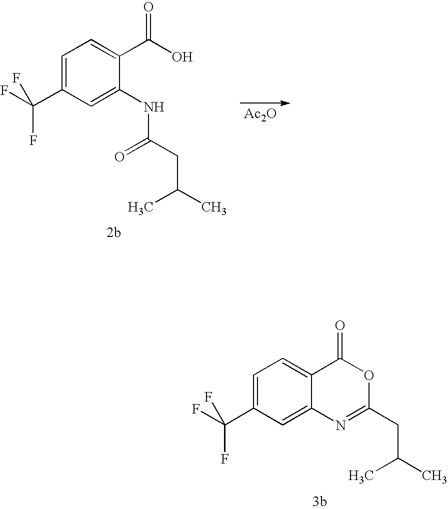

4.1 g (14.06 mol) of 2b are suspended in 20 ml of acetic anhydride and heated. The acetic acid formed is distilled off from the reaction mixture during the reaction. After 1.5 h, the reaction mixture is cooled to room temperature and evaporated to dryness in a rotary evaporator. The pasty residue is taken up in ethyl acetate, washed with NaHCO₃ solution, water and saturated NaCl solution, dried using sodium sulfate, filtered and evaporated.

Yield: 3.44 g (76%) of 3b, yellow-brown oil

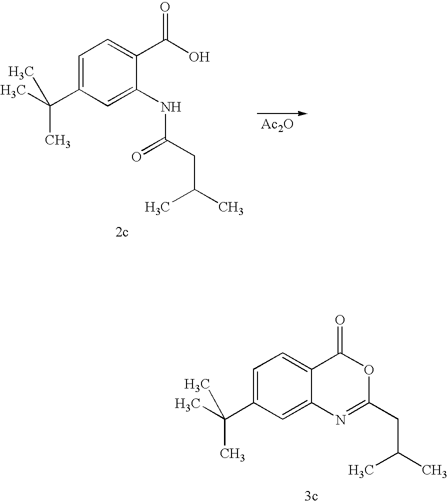

1.9 g (6.85 mol) of 2c are suspended in 32 ml of acetic anhydride and heated. The acetic acid formed is distilled off from the reaction mixture during the reaction. After 3.5 h, the reaction mixture is cooled to room temperature and evaporated to dryness in a rotary evaporator. The residue is taken up in ethyl acetate washed with NaHCO₃ solution, water and saturated NaCl solution, dried using sodium sulfate, filtered and evaporated.

Yield: 1.73 g (97%) of 3c, pale-brown solid

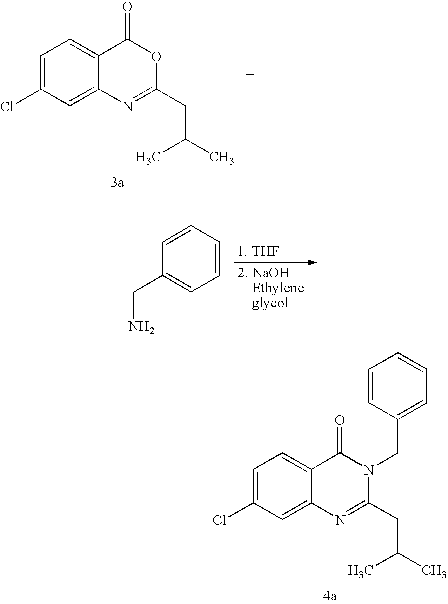

39.1 g (0.165 mol) of 3a are dissolved in 250 ml of THF, 21.7 ml (0.198 mol) of benzylamine are added, and the mixture is stirred at 65° C. for 2 h. The reaction mixture is evaporated to dryness, taken up using 140 ml of ethylene glycol, 0.66 g (0.017 mol) of NaOH is added, and the mixture is stirred at 135° C. for 1.5 h. The reaction mixture is cooled to room temperature, about 150 ml of water are added, and the mixture is neutralised using 1M HCl (pH 7-8). During this operation, the product slowly precipitated as an oil and crystallises after a short time. The crystals are filtered off with suction, washed with water, sucked dry and dried.

Yield: 53.6 g (100%) of 4a, pale-yellow crystals

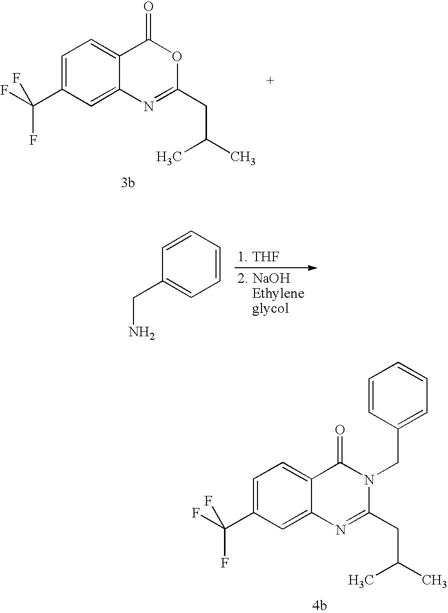

2.8 g (10.34 mmol) of 3b are dissolved in 20 ml of THF, 1.4 ml (12.8 mmol) of benzylamine are added, and the mixture is stirred at 65° C. for 2 h. The reaction mixture is evaporated to dryness, taken up using 12 ml of ethylene glycol, 285 mg (7.13 mmol) of NaOH are added, and the mixture is stirred at 135° C. for 2 h. The reaction mixture is cooled to room temperature, about 15 ml of water are added, and the mixture is neutralised using 1M HCl (pH 7-8). During this operation, the product slowly precipitated as an oil. The supernatant phase is decanted off, the oily residue is taken up in ethyl acetate, washed with water, dried using Na₂SO₄, filtered and evaporated. The residue is purified by chromatography (120 g of silica gel, eluent: petroleum ether/MTB ether-9:1).

Yield: 3.68 g (99%) of 4b, pale-brown solid

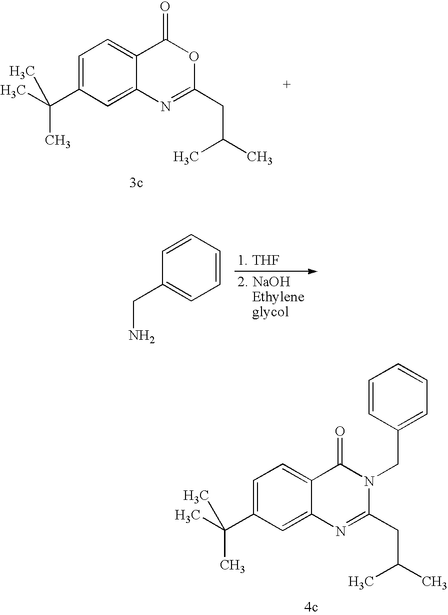

2.18 g (8.41 mmol) of 3c are dissolved in 20 ml of THF, 1.09 ml (10.33 mmol) of benzylamine are added, and the mixture is stirred at 65° C. for 1.5 h. The reaction mixture is evaporated to dryness, taken up using 15 ml of ethylene glycol, 238 mg (5.96 mmol) of NaOH are added, and the mixture is stirred at 135° C. for 3.5 h. The reaction mixture is cooled to room temperature, about 20 ml of water are added, and the mixture is neutralised using 1M HCl (pH 7-8). During this operation, the product slowly precipitated as an oil. The supernatant phase is decanted off, the oily residue is taken up in ethyl acetate, washed with water, dried using Na₂SO₄, filtered and evaporated. The residue is purified by chromatography (120 g of silica gel, eluent: petroleum ether/MTB ether-9:1).

Yield: 2.63 g (90%) of 4c, pale-brown oil

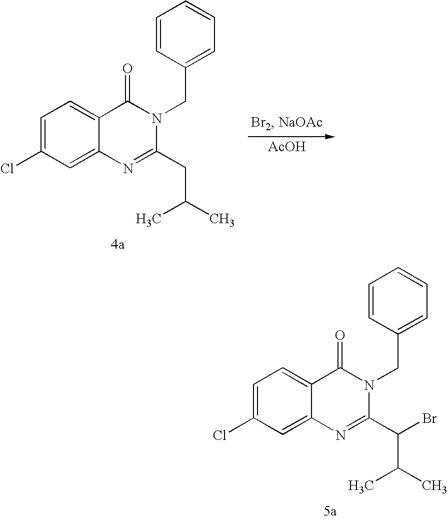

A solution of 9.1 ml of bromine (0.178 mol) in 25 ml of glacial acetic acid is slowly added dropwise at 40° C. to a solution of 53 g (0.162 mol) of 4a and 17.3 g (0.211 mol) of sodium acetate in 250 ml of glacial acetic acid, during which the temperature is held at 40-42° C. After stirring at 40° C. for 1 h, the reaction mixture is cooled to room temperature and stirred into about 2 l of water. During this operation, the product precipitated out as a tacky/viscous mass. The supernatant is decanted off, and the residue is dissolved in ethyl acetate. The solution is washed with saturated NaHCO₃ solution, water and saturated NaCl solution, dried over Na₂SO₄, filtered and evaporated.

Yield: 65 g (99%) of 5a, yellow oil

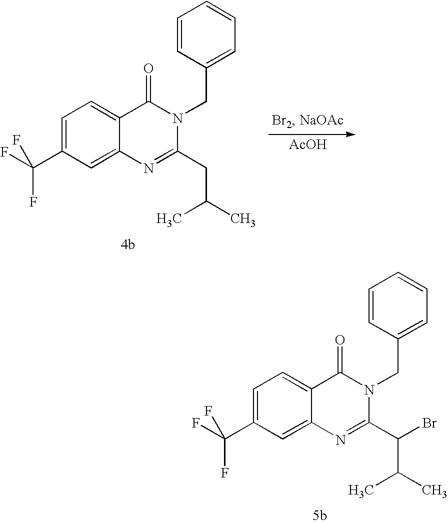

A solution of 0.58 ml of bromine (11.22 mmol) in 5 ml of glacial acetic acid is slowly added dropwise at 40° C. to a solution of 3.68 g (10.2 mmol) of 4b and 1.09 g (13.26 mmol) of sodium acetate in 15 ml of glacial acetic acid, during which the temperature is held at 40-42° C. After stirring at 40° C. for 4.5 h, the reaction mixture is cooled to room temperature, stirred into about 200 ml of water and stirred for about 1 h. The precipitate is filtered off, dissolved in ethyl acetate and washed with saturated NaHCO₃ solution, water and saturated NaCl solution, dried over Na₂SO₄, filtered and evaporated.

Yield: 4.39 g (98%) of 5b, pale-yellow crystals

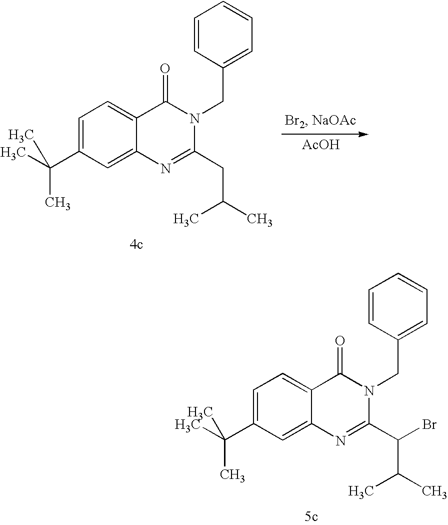

A solution of 0.42 ml of bromine (8.28 mmol) in 5 ml of glacial acetic acid is slowly added dropwise at 40° C. to a solution of 2.18 g (6.27 mmol) of 4c and 669 mg (8.15 mmol) of sodium acetate in 10 ml of glacial acetic acid, during which the temperature is held at 40-42° C. After stirring at 40° C. for 3 h, the reaction mixture is cooled to room temperature and stirred into about 200 ml of water. The precipitate is filtered off, dissolved in ethyl acetate and washed with saturated NaHCO₃ solution, water and saturated NaCl solution, dried over Na₂SO₄, filtered and evaporated. The residue is purified by chromatography (120 g of silica gel, eluent: petroleum ether/MTB ether-9:1).

Yield: 2.55 g (95%) of 5c, pale-brown oil

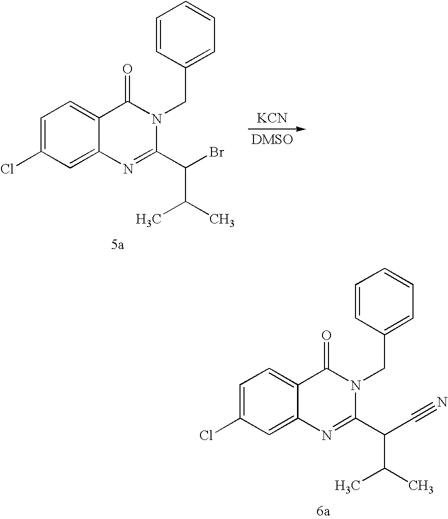

1 g (2.47 mmol) of 5a is dissolved in 20 ml of dimethyl sulfoxide, 177 mg (2.71 mmol) of potassium cyanide are added, and the mixture is stirred at 50° C. for 2 h. The solution is poured into 400 ml of water and extracted a number of times with ethyl acetate. The combined organic phases are washed with water and saturated NaCl solution, dried using Na₂SO₄, filtered and evaporated. A little diethyl ether is added to the oily residue, which is rubbed against the flask wall using a spatula until crystallisation commences. The crystallisate is filtered off with suction and rinsed with a little ether. Further product is obtained from the filtrate by chromatography (12 g of silica gel, eluent: DCM/petroleum ether-92:5).

Yield: 546 mg (63%) of 6a

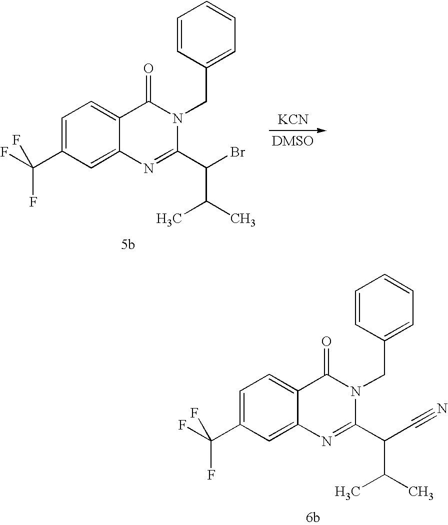

4.35 g (9.89 mmol) of 5b is dissolved in 50 ml of dimethyl sulfoxide, 709 mg (10.88 mmol) of potassium cyanide are added, and the mixture is stirred at 50° C. for 4.5 h. The solution is poured into 600 ml of water and extracted a number of times with ethyl acetate. The combined organic phases are washed with water and saturated NaCl solution, dried using Na₂SO₄, filtered and evaporated. The residue is purified by chromatography (120 g of silica gel, eluent: petroleum ether/MTB ether—9:1). The product obtained in this way is brought to crystallisation by digestion with diethyl ether, the crystallisate is filtered off with suction, rinsed with a little ether and subsequently dried in vacuo.

Yield: 2.1 g (55%) of 6b, pale-yellow crystals

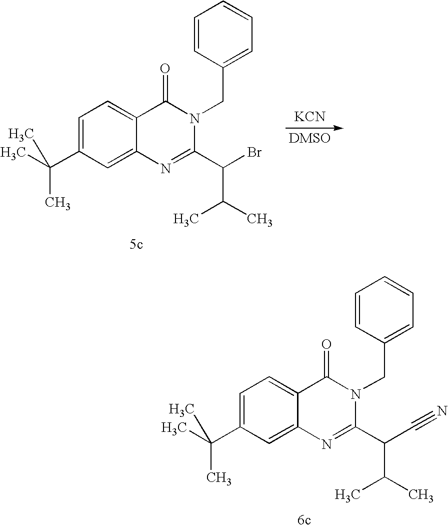

3.08 g (7.22 mmol) of 5c is dissolved in 40 ml of dimethyl sulfoxide, 517 mg (7.94 mmol) of potassium cyanide are added, and the mixture is stirred at 50° C. for 2.5 h. The solution is poured into 500 ml of water and extracted a number of times with ethyl acetate. The combined organic phases are washed with water and saturated NaCl solution, dried using Na₂SO₄, filtered and evaporated. The residue is purified by chromatography (120 g of silica gel, eluent: DCM/petroleum ether-8:2).

Yield: 1.66 g (61%) of 6c

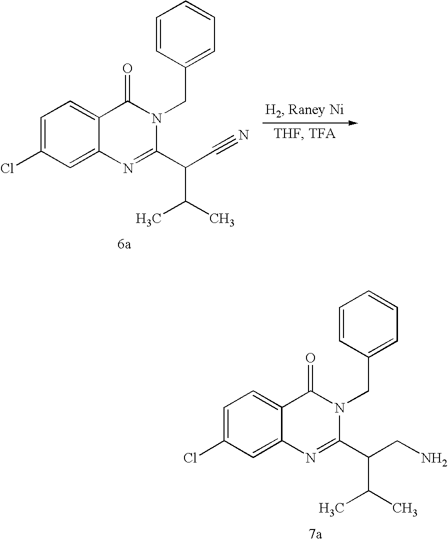

146 mg (0.41 mmol) of 6a are hydrogenated using Raney nickel in THF at 40° C. and 3 bar in the presence of trifluoroacetic acid until the reaction is complete. The reaction solution is filtered through kieselguhr, the filtrate is diluted with ethyl acetate and washed 2× with water and saturated NaHCO₃ solution. The organic phase is dried over Na₂SO₄, filtered and evaporated. The residue is purified by chromatography (12 g of silica gel, eluent: DCM, later DCM/MeOH—9:1).

Yield: 118 mg (80%) of 7a, yellow oil

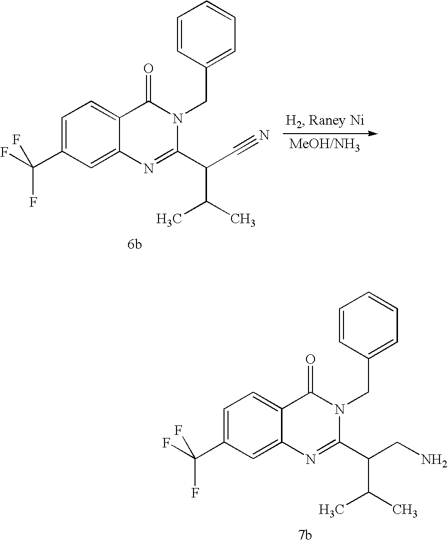

0.5 g (1.3 mmol) of 6b are hydrogenated using Raney nickel in methanolic ammonia solution at 50° C. and 5 bar until the reaction is complete. The reaction solution is filtered through kieselguhr, and the filtrate is subsequently evaporated.

Yield: 0.5 g (99%) of 7b, yellow oil

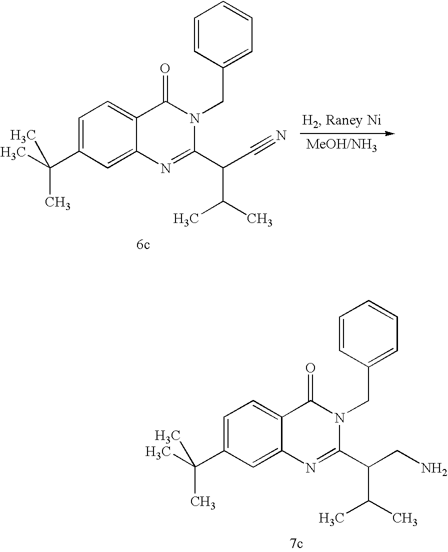

465 mg (1.25 mmol) of 6c are hydrogenated using Raney nickel in methanolic ammonia solution at 50° C. and 5 bar until the reaction is complete. The reaction solution is filtered through kieselguhr, and the filtrate is subsequently evaporated.

Yield: 448 mg (95%) of 7c, brown oil

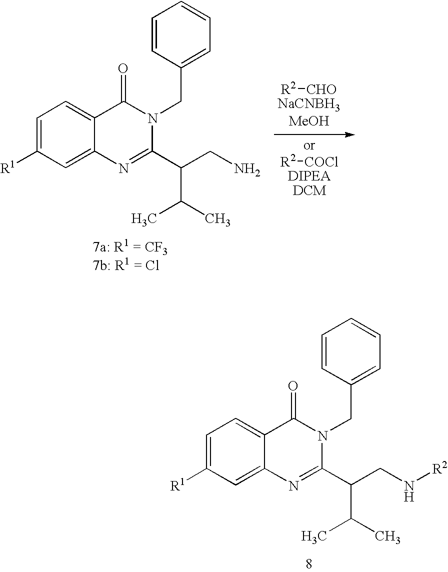

Compound 7a or 7b (1 equiv.) is dissolved in methanol together with an aldehyde (1 equiv.; R¹²—CHO), sodium cyanoborohydride (0.7 equiv.) is added, and the mixture is stirred overnight at room temperature. The reaction mixture is evaporated, the residue is dissolved in ethyl acetate and washed with saturated NaHCO₃ solution and saturated NaCl solution, dried using Na₂SO₄, filtered and evaporated. The residue is purified by flash chromatography.

8a: R¹═CF₃, R²=(CH₂)₂NH-Boc; purification: 4 g of silica gel, eluent: dichloromethane/methanol (100:0-90:10 in 40 min); yield: 40%

8b: R¹═CF₃, R²=(CH₂)₃NH-Boc; purification: 4 g of silica gel, eluent: dichloromethane/methanol (100:0-90:10 in 40 min); yield: 26%

8c: R¹═CF₃, R²=(CH₂)₂NH(CH₃)₂; purification: 4 g of silica gel, eluent: dichloromethane/methanol+1% of NH₃ 100:0-90:10 in 40 min); yield: 38%

8d: R¹═CF₃, R²═CH₂Ph; purification: 4 g of silica gel, eluent: petroleum ether/ethyl acetate (8:2-4:6 in 40 min); yield: 31%

8e: R¹═CF₃, R²=(CH₂)₂Ph; purification: 4 g of silica gel, eluent: dichloromethane/methanol (100:0-95:5 in 40 min); yield: 30%

8f: R¹=Cl, R²=(CH₂)₂NH-Boc; purification: 12 g of silica gel, eluent: dichloromethane/methanol (100:0-96:4); yield: 56%

8 g: R¹=Cl, R²=(CH₂)₃NH-Boc; purification: 12 g of silica gel, eluent: dichloromethane/methanol (100:0-96:4); yield: 46%

Compound 7b (1 equiv.) is dissolved in dichloromethane together with N-ethyldiisopropylamine (1.5 equiv.), an acid chloride (1.1 equiv.; R—COCl) is added, and the mixture is stirred at room temperature for 2 h. The reaction mixture diluted with dichloromethane, washed 2× with water and with saturated NaCl solution, dried using Na₂SO₄, filtered and evaporated. The residue is freeze-dried.

8 h: R═COPh; yield: 99%

8i: R═COCH₂Ph; yield: 99%<

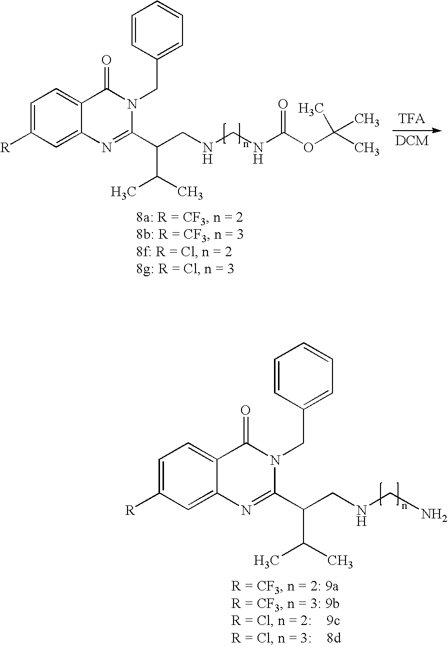

In order to remove the protecting group, 8a or 8b are stirred in DCM/TFA—2/1 at room temperature until the reaction is complete. The reaction mixture is evaporated, and the oily residue is freeze-dried overnight.

9a: R=CF₃, n=2, yield: 98%

9b: R=CF₃, n=3, yield: 99%

9c: R=Cl, n=2, yield: 95%

9d: R=Cl, n=3, yield: 99%

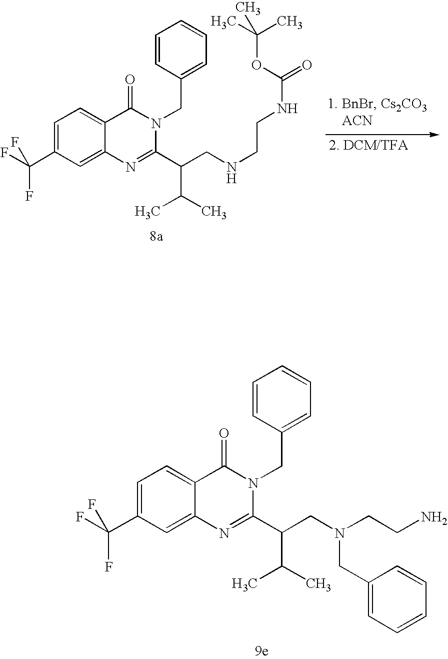

8a is initially introduced in acetonitrile at room temperature, caesium carbonate (1.1 equiv.) is added, and benzyl bromide (1 equiv.) is subsequently slowly added dropwise. The reaction mixture is stirred overnight at room temperature, diluted with ethyl acetate and washed 2× with water. The organic phase is dried using Na₂SO₄, filtered and evaporated. The residue is by flash chromatography (4 g of silica gel, eluent: petroleum ether/ethyl acetate 98:2-90:10 in 45 min).

Yield: 63%

Removal of the Boc protecting group analogously to 9a-d; yield: 87%

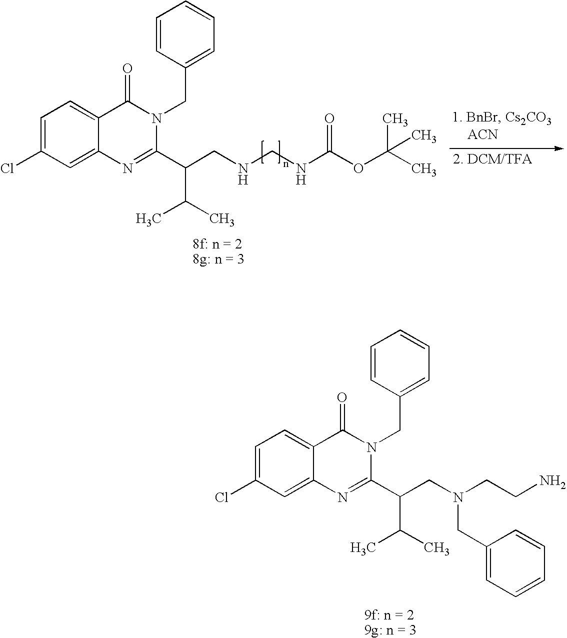

8f,g are reacted and worked up analogously to the preparation of 9e.

9f: n=2, step 1, yield: 62%; removal of the Boc protecting group, yield: 96%

9 g: n=3, step 1, yield: 53%; removal of the Boc protecting group, yield: 99%

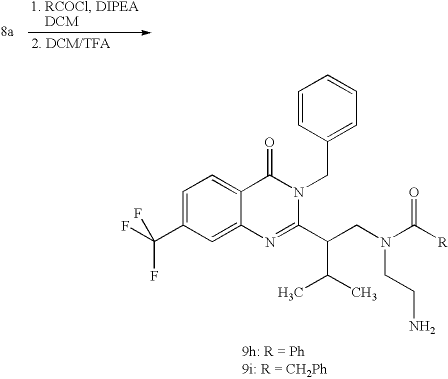

9 h: 8a is reacted and worked up analogously to the preparation of 8 h. Yield: 99%

Removal of the Boc protecting group analogously to 9a,b; yield: 84%

9i: 8a is reacted and worked up analogously to the preparation of 8i. Yield: 98%

Removal of the Boc protecting group analogously to 9a,b; yield: 100%

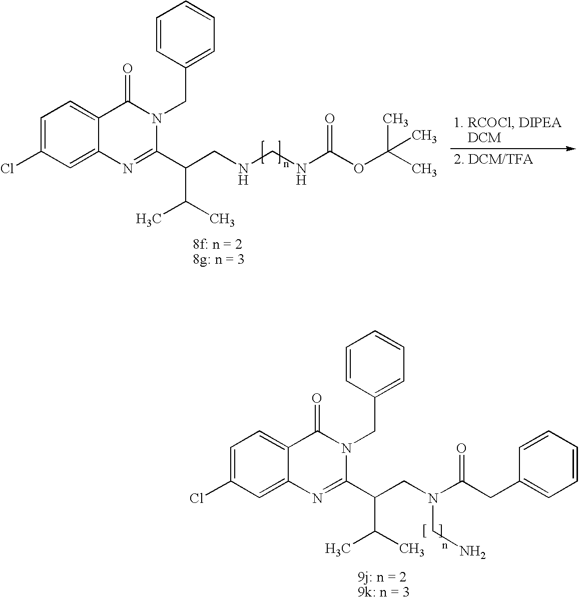

Reaction of 8f,g analogously to the preparation of 8i.

9j: n=2, step 1, yield: 50%; removal of the Boc protecting group, yield: 80%

9k: n=3, step 1, yield: 93%; removal of the Boc protecting group, yield: 86%

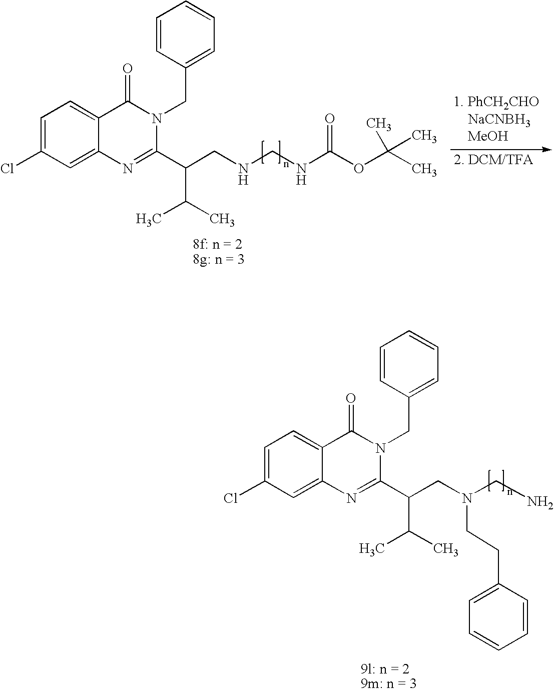

Compound 8f or 8 g (1 equiv.) is dissolved in methanol together with phenylacetaldehyde (1.1 equiv.), sodium cyanoborohydride (0.7 equiv.) is added, and the mixture is stirred overnight at room temperature. The reaction mixture is evaporated, the residue is dissolved in ethyl acetate and washed with saturated NaHCO₃ solution and saturated NaCl solution, dried using Na₂SO₄, filtered and evaporated. The residue is purified by flash chromatography.

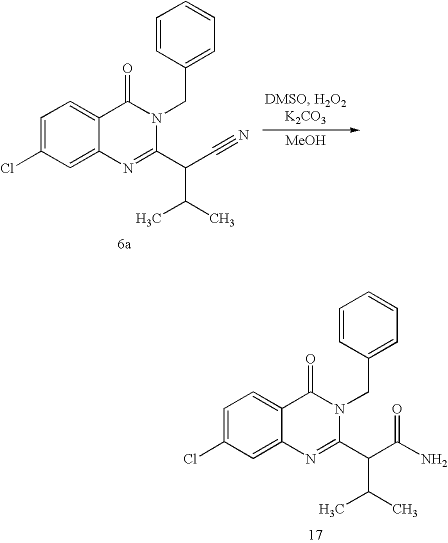

200 mg (0.57 mmol) of 6a and 393 mg (2.84 mmol) of potassium carbonate are suspended in 10 ml of methanol, 0.141 ml (1.99 mmol) of dimethyl sulfoxide and 0.291 ml (2.84 mmol) of hydrogen peroxide are added at room temperature, and the mixture is stirred for 30 min. The suspension is filtered with suction and rinsed with a little MeOH and water. The crystallisate is dissolved in ethyl acetate and DCM, dried (Na₂SO₄), filtered and evaporated (80 mg). The aqueous filtrate is extracted three times with ethyl acetate, the combined organic phases are washed with water, dried and evaporated. The is triturated with MeOH, filtered off with suction and rinsed with a little MeOH (40 mg).

Yield: 120 mg (57%) of 17, colourless crystals

Further compounds according to the invention are obtained analogously using the corresponding precursors.

For example, the following compounds (19-22) are advantageously obtained in accordance with the following reaction schemes starting from precursors 18a-c:

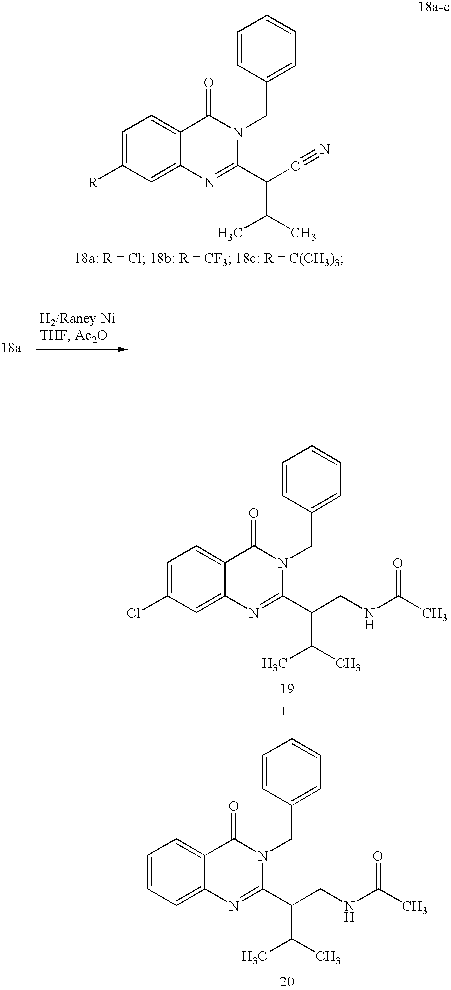

Compounds 19 and 20 obtained in accordance with the above reaction scheme can be separated and characterised by HPLC chromatography (column: Chromolith SpeedROD RP18e 50-4.6; gradient 5.5 min./flow rate: 2.75 ml (90:10-0:100 (H₂O+0.01% by vol. of TFA: CH₃CN+0.01% by vol. of TFA)); wavelength 220 nm; 19: Rt (retention time)=2.79 min; 20: Rt=2.41 min).

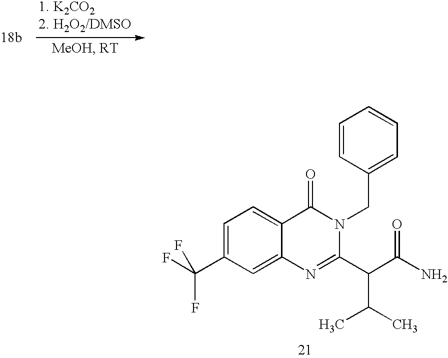

Compound 21 obtained in accordance with the above reaction scheme can be characterised by HPLC chromatography* (Rt=2.91 min) and by mass spectroscopy (ESI-MS: M+1=404.2).

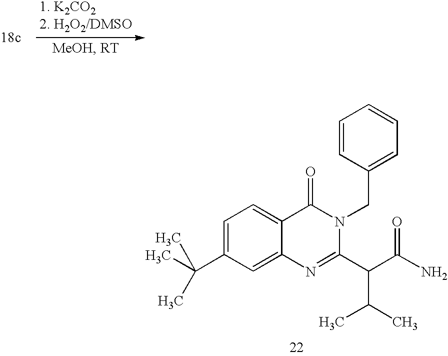

Compound 22 obtained in accordance with the above reaction scheme can be characterised by HPLC chromatography* (Rt=3.05 min).

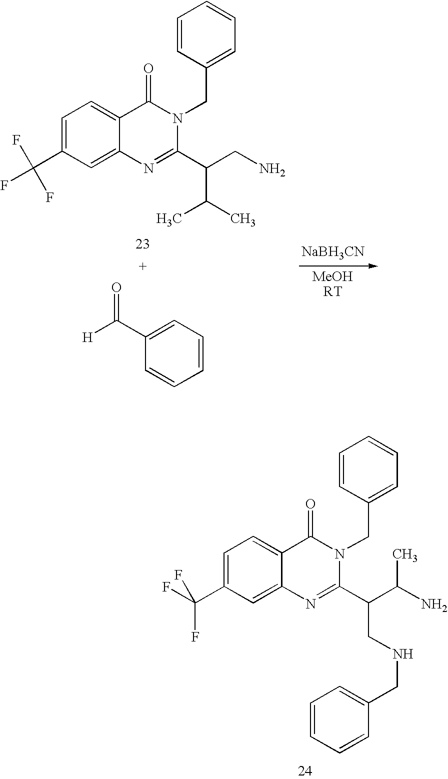

Compound 24 obtained in accordance with the above reaction scheme can be characterised by HPLC chromatography* (Rt=2.60 min) and by mass spectroscopy (ESI-MS: M+1=480.15). *: HPLC chromatography by the following method:

column: Chromolith SpeedROD RP18e 50-4.6;

gradient 5.5 min./flow rate: 2.75 ml (90:10-0:100 (H₂O+0.01% by vol. of

TFA: CH₃CN+0.01% by vol. of TFA));

Wavelength 220 nm.

### Example A

### Assay I

The efficacy of the compounds according to the invention can be determined, for example, via the Eg5 ATPase activity, which is measured via an enzymatic regeneration of the product ADP to ATP by means of pyruvate kinase (PK) and subsequent coupling to an NADH-dependent lactate dehydrogenase (LDH) reaction. The reaction can be monitored via the change in absorbance at 340 nm by coupling to the NADH-dependent LDH. The regeneration of the ATP simultaneously ensures that the substrate concentration remains constant. The change in absorbance per time unit are analysed graphically and a linear regression carried out in the visually linear region of the reaction.

### Example B

### Assay II

The combination of the antiprotozoic pentamidine and the inhibitors of kinesin ATPase Eg5/KSP results in increased inhibitory effects in cell proliferation tests with the colon carcinoma cell line HCT116.

Eg5 inhibitors adversely affect the ATPase activity and inhibit the course of the cell cycle owing to an error in the separation of the spindle poles.

The determination of the efficacy of the compounds of the formula I according to the invention in combination with compounds of the formula VI and/or medicaments from Table I can be demonstrated as follows in combination assays:

10³ to 10⁴ cells of a defined cell line (HCT116, Colo 205, MDA-MB 231, etc.) are sown into each well of a 96-well microtitre plate and cultivated overnight under standard conditions. For the substances of the combination to be tested, 10-50 mM stock solutions in DMSO were prepared. Dilution series (generally 3-fold dilution steps) of the individual substances were combined with one another in the form of a pipetting scheme (see scheme below), while maintaining a DMSO final concentration of 0.5% (v/v). Next morning, the substance mixtures were added to the cells, which were incubated under culture conditions for a further 48 hours. At the end of the cultivation, Crystal Violet staining of the cells was carried out. After extraction of the Crystal Violet from the fixed cells, the absorption at 550 nm was measured spectrophotometrically. It can be used as a quantitative measure of the adherent cells present.

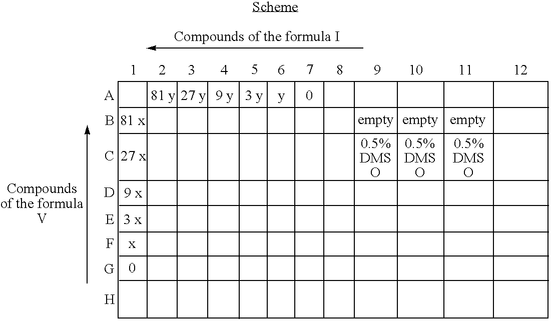

The following examples relate to medicaments:

### Example C

### Injection Vials

A solution of 100 g of an active ingredient of the formula I and 5 g of disodium hydrogenphosphate in 3 l of bidistilled water is adjusted to pH 6.5 using 2 N hydrochloric acid, sterile filtered, transferred into injection vials, lyophilised under sterile conditions and sealed under sterile conditions. Each injection vial contains 5 mg of active ingredient.

### Example D

### Suppositories

A mixture of 20 g of an active ingredient of the formula I with 100 g of soya lecithin and 1400 g of cocoa butter is melted, poured into moulds and allowed to cool. Each suppository contains 20 mg of active ingredient.

### Example E

### Solution

A solution is prepared from 1 g of an active ingredient of the formula I, 9.38 g of NaH₂PO₄.2H₂O, 28.48 g of Na₂HPO₄.12H₂O and 0.1 g of benzalkonium chloride in 940 ml of bidistilled water. The pH is adjusted to 6.8, and the solution is made up to 1 l and sterilised by irradiation. This solution can be used in the form of eye drops.

### Example F

### Ointment

500 mg of an active ingredient of the formula I are mixed with 99.5 g of Vaseline under aseptic conditions.

### Example G

### Tablets

A mixture of 1 kg of active ingredient of the formula I, 4 kg of lactose, 1.2 kg of potato starch, 0.2 kg of talc and 0.1 kg of magnesium stearate is pressed in a conventional manner to give tablets in such a way that each tablet contains 10 mg of active ingredient.

### Example H

### Dragees

Tablets are pressed analogously to Example E and subsequently coated in a conventional manner with a coating of sucrose, potato starch, talc, tragacanth and dye.

### Example I

### Capsules

2 kg of active ingredient of the formula I are introduced into hard gelatine capsules in a conventional manner in such a way that each capsule contains 20 mg of the active ingredient.

### Example J

### Ampoules

A solution of 1 kg of active ingredient of the formula I in 60 l of bidistilled water is sterile filtered, transferred into ampoules, lyophilised under sterile conditions and sealed under sterile conditions. Each ampoule contains 10 mg of active ingredient.

1. A compound of formula I 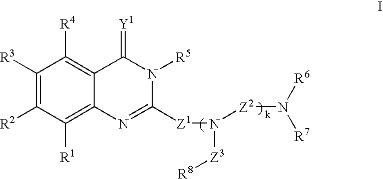
in which
R¹, R², R³ and R⁴, independently of one another, are H, A, Ar, Het, OR^{a}, SR^{a}, OAr, SAr, N(R^{a})₂, NR^{a}Ar, Hal, NO₂, CN, (CH₂)ₘCOOR^{a}, (CH₂)ₘCOOAr, (CH₂)ₘCON(R^{a})₂, (CH₂)ₘCONHAr, COR^{a}, COAr, S(O)ₘA, S(O)ₘAr, NHCOA, NHCOAr, NHSO₂A, NHSO₂Ar or SO₂N(R^{a})₂,
R^{a} is H, A, Ar, Het, aralkyl or heteroaralkyl,
R⁵, R⁸, independently of one another, are H, A, Ar, Het, aralkyl or heteroaralkyl,
R⁶, R⁷, independently of one another, are H, A, Ar, Het, aralkyl or heteroaralkyl, or
R⁶ and R⁷, together with the N atom to which they are bonded, form a saturated or unsaturated 5-, 6- or 7-membered heterocycle, which may optionally contain 1, 2 or 3 further heteroatoms selected from the group consisting of N, S and O,
Y¹ is O, S or NR¹,
Z¹, Z², independently of one another, are selected from the group consisting of (CR⁹R¹⁰)ₙ and (CR⁹R¹⁰)ₚ—(C═Y²)—(CR¹¹R¹²)_{q},
Z³ is absent or is selected independently from the meanings indicated for Z¹ and Z²,
A is alkyl or cycloalkyl,
Ar is aryl or heteroaryl,
Het is heteroaryl or heterocyclyl,
Hal is F, Cl, Br or I,
Y² is O, S or NR²,
R⁹, R¹⁰, R¹¹, R¹², independently of one another, are H, A, OA, Ar, Het, aralkyl or heteroaralkyl,
k is 0, 1 or 2,
m is 1, 2, 3 or 4,
n is 1, 2, 3, 4, 5 or 6,
p, q, independently of one another, denote 0, 1, 2, 3 or 4,
and pharmaceutically usable derivatives, solvates, tautomers, salts stereoisomers and mixtures thereof in all ratios.

2. The compound according to claim 1, selected from the group consisting of the compound of formula Iα 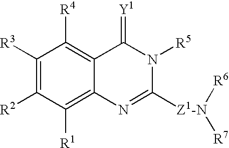
in which
R¹, R², R³, R⁴, R⁵, R⁶, R⁷, Y¹ and Z¹ are selected independently of one another from the meanings indicated in claim 1;
and the compound of formula Iβ 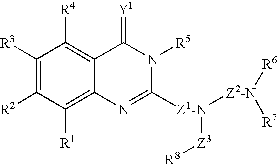
in which
R¹, R², R³, R⁴, R⁵, R⁶, R⁷, R⁸, Y¹, Z¹, Z² and Z³ have the meanings indicated in claim 1;
and pharmaceutically usable derivatives, solvates, tautomers, salts stereoisomers and mixtures thereof in all ratios.

3. The compound according to claim 1 in which
R² is A, CF₃, OCF₃, SA, SCN, CH₂CN, —OCOA, Hal, SCF₃, t-butyl, —CH(CH₃)CH₂CH₃, isopropyl, ethyl or methyl;
and
R³ is A, CF₃, OCF₃, SA, SCN, CH₂CN, —OCOA, Hal, SCF₃, t-butyl, —CH(CH₃)CH₂CH₃, isopropyl, ethyl or methyl.

4. The compound according to claim 1 in which
R¹ and R⁴, independently of one another, either denote are H or are selected from the group consisting of A, CF₃, OCF₃, OR^{a}, SA, S(O)₂A, S(O)A, CH₂CN, COOA, CONHA, Hal, SCF, CN and Het.

5. The compound according to claim 1 in which
R⁵ is selected from the group consisting of Ar, aralkyl and heteroaralkyl,
R⁶, R⁷, independently of one another, are selected from the group consisting of H, A, Ar and aralkyl, and
R⁸ is selected from the group consisting of H, A, Ar and Het.

6. The compound according to claim 1 in which
R⁵ is unsubstituted or substituted benzyl, and
R⁸ is unsubstituted or substituted phenyl.

7. The compound according to claim 1, selected from the group consisting of the sub-formulae IA to IR: 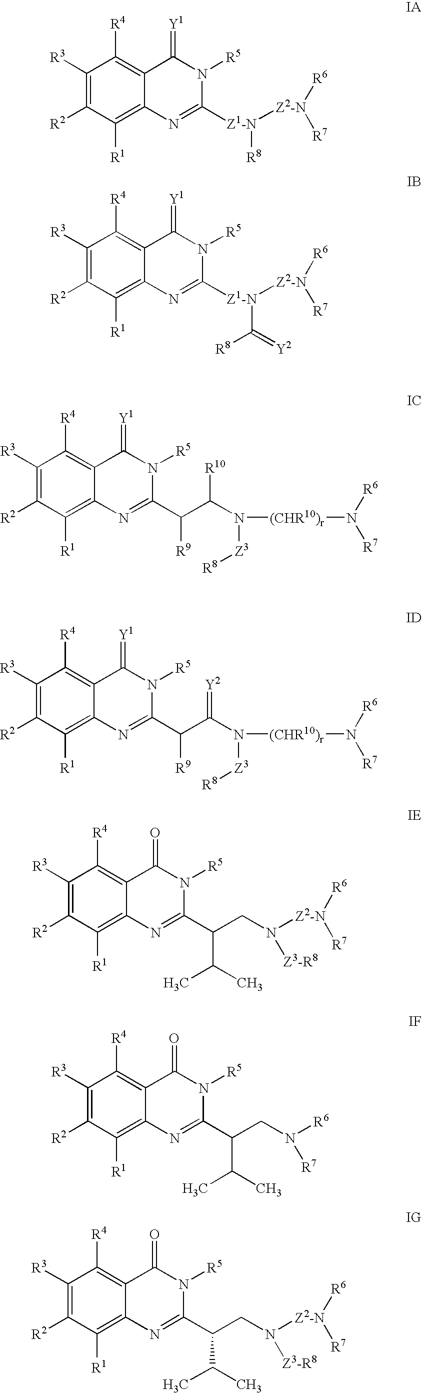 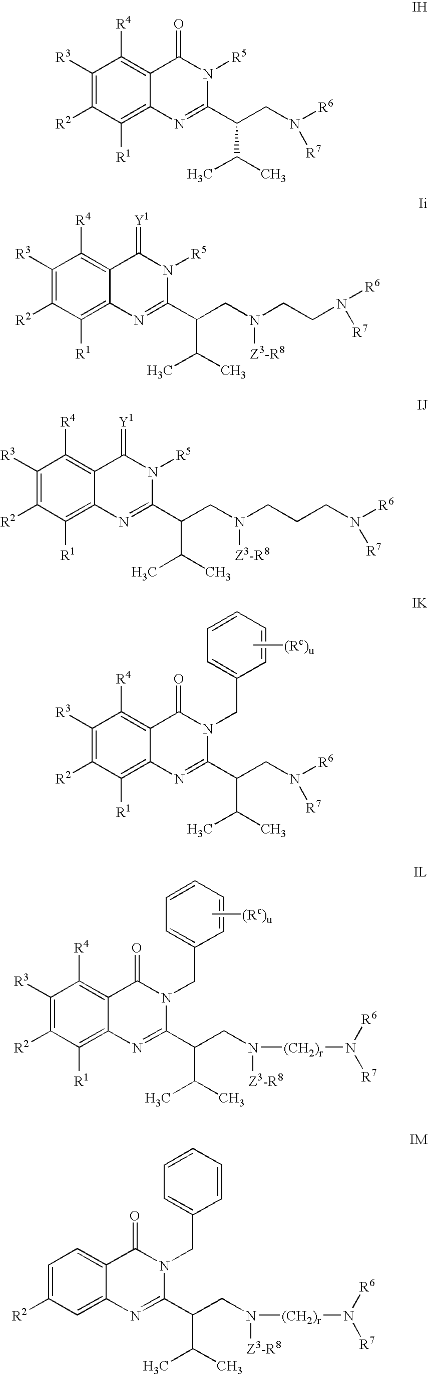 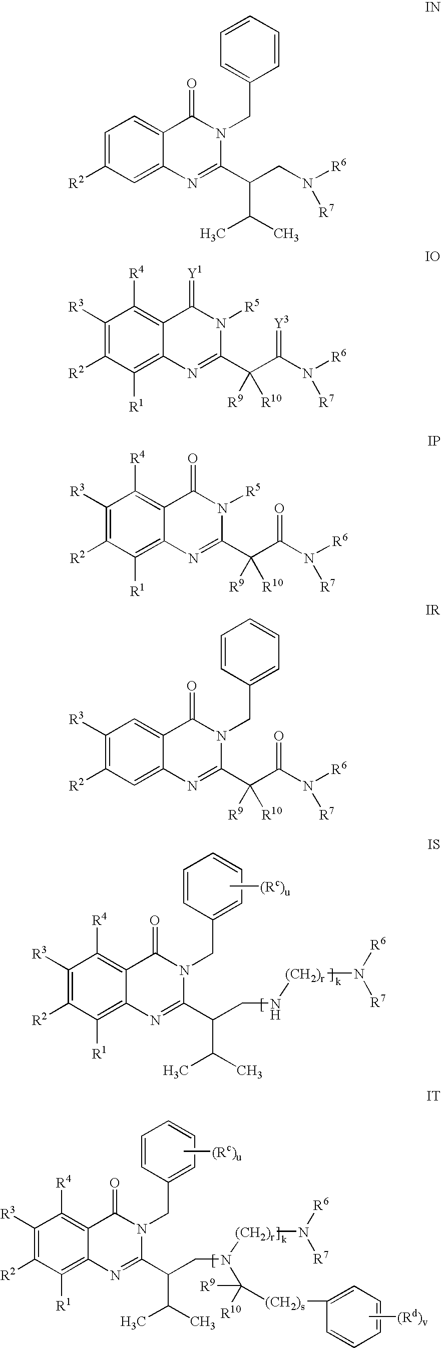
in which
R¹, R², R³, R⁴, R⁵, R⁶, R⁷, R⁸, R⁹, R¹⁰, Y¹, Y², Z¹ and Z³ independently of one another have the meanings indicated in claim 1,
r is 1, 2, 3 or 4,
s and t, independently of one another, are 0, 1 or 2,
Y³ is O, S or NR^{a},
R^{c} and R^{d}, independently of one another, are selected from the meanings indicated for R¹, R², R³ and R⁴, and
u and v, independently of one another, are 0, 1, 2 or 3,
and pharmaceutical usable derivatives, solvates, tautomers, salts stereoisomers and mixtures thereof in all ratios.

8. The compound according to claim 1, selected from the group consisting of:
2-(1-{[(2-aminoethyl)benzylamino]methyl}-2-methylpropyl)-3-benzyl-7-trifluoromethyl-3H-quinazolin-4-one;
3-benzyl-2-[1-(benzylaminomethyl)-2-methylpropyl]-7-trifluoromethyl-3H-quinazolin-4-one;
2-{1-[(2-aminoethylamino)methyl]-2-methylpropyl}-3-benzyl-7-trifluoromethyl-3H-quinazolin-4-one;
N-(2-aminoethyl)-N-[2-(3-benzyl-4-oxo-7-trifluoromethyl-3,4-dihydroquinazolin-2-yl)-3-methylbutyl]benzamide;
N-[2-(3-benzyl-4-oxo-7-trifluoromethyl-3,4-dihydroquinazolin-2-yl)-3-methylbutyl]benzamide;
N-[2-(3-benzyl-4-oxo-7-trifluoromethyl-3,4-dihydroquinazolin-2-yl)-3-methylbutyl]-2-phenylacetamide;
N-(2-aminoethyl)-N-[2-(3-benzyl-4-oxo-7-trifluoromethyl-3,4-dihydroquinazolin-2-yl)-3-methylbutyl]-2-phenylacetamide;
2-{1-[(3-aminopropylamino)methyl]-2-methylpropyl}-3-benzyl-7-trifluoromethyl-3H-quinazolin-4-one;
3-benzyl-2-{1-[(2-dimethylaminoethylamino)methyl]-2-methylpropyl}-7-trifluoromethyl-3H-quinazolin-4-one;
3-benzyl-2-[2-methyl-1-(phenethylaminomethyl)propyl]-7-trifluoromethyl-3H-quinazolin-4-one;
2-{1-[(2-aminoethylamino)methyl]-2-methylpropyl}-3-benzyl-7-chloro-3H-quinazolin-4-one;
N-(2-aminoethyl)-N-[2-(3-benzyl-7-chloro-4-oxo-3,4-dihydroquinazolin-2-yl)-3-methylbutyl]-2-phenylacetamide;
2-(1-{[(2-aminoethyl)benzylamino]methyl}-2-methylpropyl)-3-benzyl-7-chloro-3H-quinazolin-4-one;
2-(1-{[(2-aminoethyl)phenethylamino]methyl}-2-methylpropyl)-3-benzyl-7-chloro-3H-quinazolin-4-one;
2-{1-[(3-aminopropylamino)methyl]-2-methylpropyl}-3-benzyl-7-chloro-3H-quinazolin-4-one;
N-(3-aminopropyl)-N-[2-(3-benzyl-7-chloro-4-oxo-3,4-dihydroquinazolin-2-yl)-3-methylbutyl]-2-phenylacetamide;
2-(1-{[(3-aminopropyl)benzylamino]methyl}-2-methylpropyl)-3-benzyl-7-chloro-3H-quinazolin-4-one;
2-(1-{[(3-aminopropyl)phenethylamino]methyl}-2-methylpropyl)-3-benzyl-7-chloro-3H-quinazolin-4-one;
2-(1-aminomethyl-2-methylpropyl)-3-benzyl-6,7-dichloro-3H-quinazolin-4-one;
2-(1-aminomethyl-2-methylpropyl)-3-benzyl-7-chloro-6-fluoro-3H-quinazolin-4-one;
2-(1-aminomethyl-2-methylpropyl)-3-benzyl-7-chloro-6-methyl-3H-quinazolin-4-one;
2-(3-benzyl-7-chloro-4-oxo-3,4-dihydroquinazolin-2-yl)-3-methylbutyramide;
2-(1-aminomethyl-2-methylpropyl)-3-benzyl-3H-quinazolin-4-one;
2-(1-aminomethyl-2-methylpropyl)-3-benzyl-7-chloro-3H-quinazolin-4-one;
2-(1-aminomethyl-2-methylpropyl)-3-benzyl-7-tert-butyl-3H-quinazolin-4-one;
2-(1-aminomethyl-2-methylpropyl)-3-benzyl-7-trifluoromethyl-3H-quinazolin-4-one;
N-[2-(3-benzyl-7-chloro-4-oxo-3,4-dihydroquinazolin-2-yl)-3-methylbutyl]acetamide;
N-[2-(3-benzyl-4-oxo-3,4-dihydroquinazolin-2-yl)-3-methylbutyl]acetamide;
2-(3-benzyl-4-oxo-7-trifluoromethyl-3,4-dihydroquinazolin-2-yl)-3-methylbutyramide;
2-(3-benzyl-7-tert-butyl-4-oxo-3,4-dihydroquinazolin-2-yl)-3-methylbutyramide;
3-benzyl-2-[1-(benzylaminomethyl)-2-methylpropyl]-7-trifluoromethyl-3H-quinazolin-4-one;
and pharmaceutically tolerated derivatives, solvates, salts stereoisomers and mixtures thereof in all ratios.

9. A process for the preparation of compounds of the formula I according to claim 1 and pharmaceutically usable derivatives, salts, solvates, tautomers and stereoisomers thereof, characterised in that
a) a compound of the formula II 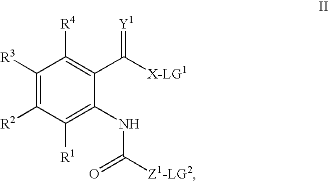
in which R¹, R², R³, R⁴, R⁵, Y¹ and Z¹ have the meanings indicated in claim 1, X stands for O NH or S, and LG¹ and LG² each stands for a leaving group, is cyclised with removal of the leaving group LG¹ to give a compound of formula IIb 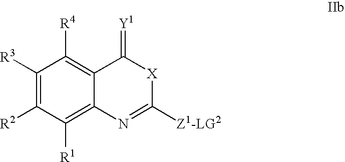
b) the compound of the formula IIb is reacted with a compound of the formula III 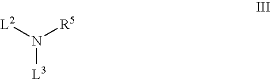
in which R⁵ has the meaning indicated in claim 1, and L² and L³, independently of one another, stand for H or a metal atom,
giving a compound of formula IIc 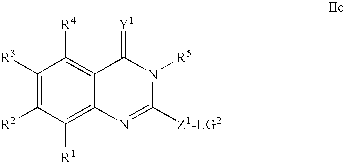
c) the compound of the formula IIc is reacted with a compound of the formula IV 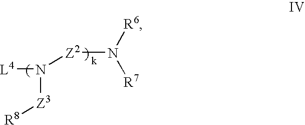
in which L⁴ stands for H or a metal atom, and Z², Z³, k, R⁶, R⁷ and R⁸ have the meanings indicated in one of claims 1 to 4,
where the groups LG² and L⁴ are removed, giving a compound of the formula I; and optionally
d) the resultant compound of the formula I is isolated and/or treated with an acid or base in order to convert it into one of its salts.

10. A process for the preparation of compounds according to one of claim 1 and pharmaceutically usable derivatives, salts, solvates, tautomers and stereoisomers thereof, characterised in that
a) a compound of formula II′ 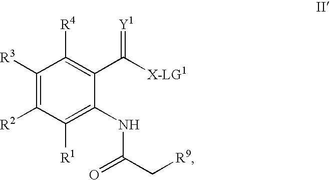
in which R¹, R², R³, R⁴, R⁵, R⁹, X and Y¹ have the meanings indicated in claim 1, LG¹ stands for a leaving group,
is cyclised with removal of the leaving group LG¹ to give a compound of formula IIb′ 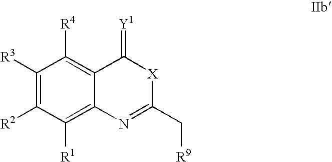
b) the compound of the formula IIb′ is converted by reaction with a compound of formula III 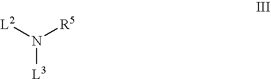
and introduction of a leaving group LG³, into a compound of formula IIc′ 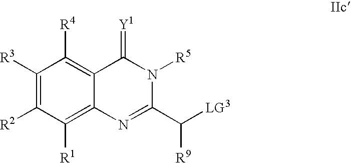
c1) the compound of the formula IIc′ is converted by reaction with cyanide into a compound of formula IId′ 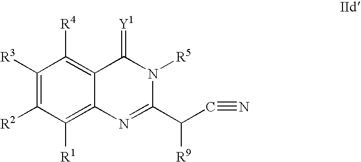
c2) the compound of the formula IId′ is converted under reductive conditions into a compound of formula I′ 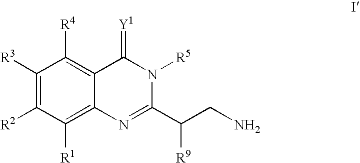
and optionally either
c3a) the compound of the formula I′ obtained in step c2) is converted by reaction with a compound FG¹-R⁶ and/or FG²-R⁷ into a compound of the formula I″ which is a compound of the formula I in which k is equal to 0, Z¹ stands for —CHR⁹—CH₂— and in which R⁶ and/or R⁷ are different from H 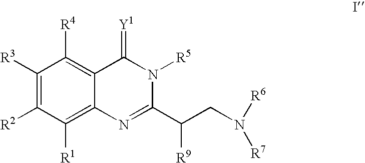
or c3b) the compound of the formula I′ obtained in step c2) is converted by reaction with a compound of formula FG³-Z²-NR⁶R⁷ and optionally a compound FG⁴-Z³-R⁸, in which FG³ and FG⁴ each stands for a functional group, into a compound of formula I′″ 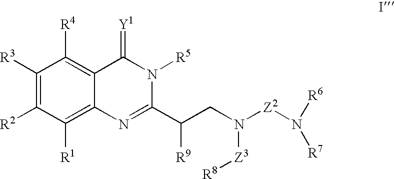
and optionally
d) the compound according to claim 1 obtained in process step c2), C3a) or c3b) is isolated and/or treated with an acid or base in order to convert it into one of its salts.

11. A medicament comprising at least one compound of the formula I according to claim 1 and/or pharmaceutically usable derivatives, salts, solvates, tautomer, stereoisomers and mixtures thereof in all ratios, and optionally excipients and/or adjuvants.

12. A mixture comprising one or more compounds of the formula I and an amount of one or more compounds of formula VI, analogues thereof and/or metabolites thereof 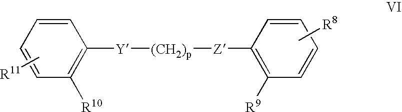
in which
Y′ and Z′ each, independently of one another, denote O or N, R⁹ and R¹⁰ each, independently of one another, denote H, OH, halogen, OC1-10-alkyl, OCF₃, NO₂ or NH₂, n denotes an integer 2, 3, 4, 5 or 6, and 6, each inclusive, and R⁸ and R¹¹ are each, independently of one another, in the meta- or para-position and are selected from the group consisting of:

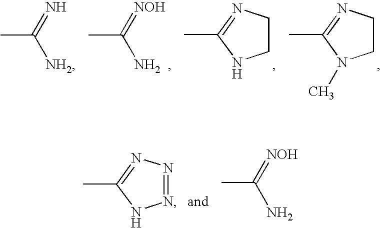

13. A mixture according to claim 12, where the compound of the formula VI is pentamidine or salts thereof.

14. A method comprising treating a disease or diseases in a human or animal with a compounds according to claim 1 or pharmaceutically usable derivatives, salts, solvates, tautomers, stereoisomers or mixtures thereof in all ratios.

15. The method according to claim 14, characterised in that the diseases can be influenced by the inhibition, regulation and/or modulation of the mitotic motor protein Eg5.

16. The method according to claim 14 wherein the diseases are cancer diseases.

17. The method according to claim 16, where the cancer diseases are accompanied by a tumour selected from the group consisting of squamous epithelium, bladder, stomach, kidneys, head and neck, esophagus, cervix, thyroid, intestine, liver, brain, prostate, urogenital tract, lymphatic system, stomach, larynx and/or lung tumours.

18. The method according to claim 17, where the tumour originates from the group consisting of lung adenocarcinoma, small-cell lung carcinomas, pancreatic cancer, glioblastomas, breast carcinoma and colon carcinoma.

19. The method of according to claim 16, where the cancer diseases are blood and immune system cancer diseases.

20. The method according to claim 19, where the cancer is selected from the group consisting of monocytic leukaemia, acute myeloid leukaemia, chronic myeloid leukaemia, acute lymphatic leukaemia and/or chronic lymphatic leukaemia.

21. A method comprising treating a cancer in a human or animal with the compounds of the formula I according to claim 1 and/or physiologically acceptable salts and solvates thereof in combination with a therapeutically effective amount of one or more compounds of the formula VI, analogues thereof and/or metabolites thereof. 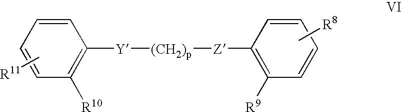
in which
Y′ and Z′ each, independently of one another, denote O or N, R⁹ and R¹⁰ each, independently of one another, denote H, OH, halogen, OC1-10-alkyl, OCF₃, NO₂ or NH₂, n denotes an integer between 2, 3, 4, 5 or 6 and R⁸ and R¹¹ are each, independently of one another, in the meta- or para-position and are selected from the group: 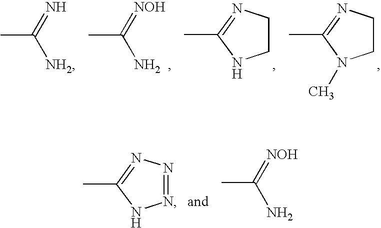
where
the compounds of the formula I and the compounds of the formula VI, analogues thereof and/or metabolites thereof are administered simultaneously or within 14 days of one another in amounts which are sufficient to inhibit the growth of a tumour or of other hyperproliferative cells.

22. The method according to claim 21, where the compound of the formula VI is pentamidine or salts thereof.

23. The method of claim 14, wherein the diseases are cancer and where a therapeutically effective amount of a compound of the formula I is administered in combination with radiotherapy and a compound from the group consisting of 1) oestrogen receptor modulator, 2) androgen receptor modulator, 3) retinoid receptor modulator, 4) cytotoxic agent, 5) antiproliferative agent, 6) prenyl-protein transferase inhibitor, 7) HMG-COA reductase inhibitor, 8) HIV protease inhibitor, 9) reverse transcriptase inhibitor and 10) angiogenesis inhibitors.
